SEQUENCE LISTING (1 sequence)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2025/0025876 A1
(43) Pub. Date: Jan. 23, 2025

(54) FLUID CONTROL IN MICROFLUIDIC DEVICES

(71) Applicant: LumiraDx UK Ltd., London (GB)

(72) Inventors: Aman Murtaza Khan, Bedford (GB); Badr Aman Khan, Bedford (GB); Brian McGuigan, Doune, Stirling (GB); David William Taylor, Alloa (GB); David Kinniburgh Lang, Glasglow (GB); John Iain William Deane, Dollar (GB); Lois Bello Fernandez De Sanmamed, Stirling (GB); Michael Flett, Cumbernauld (GB); Phill Lowe, Tullibody (GB); Steven Alexander Keatch, Stirling (GB); Usman Ali Khan, Bedford (GB); David Scott, Oxford (GB); Thomas J. Quinlan, Sturbridge, MA (US); Nigel Malcolm Lindner, Bedford (GB); Marcus Twomey, Stirling (GB); Graeme John McInnes, Dunfermline (GB)

(21) Appl. No.: 18/826,892

(22) Filed: Sep. 6, 2024

Related U.S. Application Data
(63) Continuation of application No. 17/860,933, filed on Jul. 8, 2022.
Application No. 17/860,933 is a continuation of application No. PCT/US2021/013325, filed on Jan. 13, 2021.
(60) Provisional application No. 63/092,371, filed on Oct. 15, 2020.
(60) Provisional application No. 63/067,782, filed on Aug. 19, 2020.
(60) Provisional application No. 63/055,744, filed on Jul. 23, 2020.
(60) Provisional application No. 63/032,410, filed on May 29, 2020.
(60) Provisional application No. 62/991,446, filed on Mar. 18, 2020.
(60) Provisional application No. 62/972,921, filed on Feb. 11, 2020.
(60) Provisional application No. 62/960,421, filed on Jan. 13, 2020.

(51) Int. Cl. B01L 3/00; B01F 33/30; B01F 33/40; B01F 101/23; G01N 33/533; G01N 33/543; G01N 33/569

(52) U.S. Cl. CPC B01L 3/50273; B01F 33/304; B01F 33/401; B01L 3/502753; G01N 33/533; G01N 33/54326; G01N 33/5438; G01N 33/56983; B01F 2101/23; B01L 2200/16; B01L 2400/043; B01L 2400/0436; B01L 2400/0481; G01N 2333/165

(57) ABSTRACT

A diagnostic system for determining the presence of a target in a sample liquid that includes a diagnostic reader and a microfluidic strip having a microfluidic channel network therein. An actuator within the reader modifies the pressure of a gas in gaseous communication with a liquid-gas interface of a sample liquid within the microfluidic channel network to move and/or mix the sample liquid. The pressure modifications may be continuous and/or oscillatory.

### RELATED APPLICATIONS

The present application is a continuation of U.S. patent application Ser. No. 17/860,933, filed on Jul. 8, 2022, which is a continuation of International Application No. PCT/US2021/013325, filed on Jan. 13, 2021, which claims the benefit of and priority to U.S. Provisional Application No. 63/092,371, filed on Oct. 15, 2020, U.S. Provisional Application No. 63/067,782, filed on Aug. 19, 2020, U.S. Provisional Application No. 63/055,744, filed on Jul. 23, 2020, U.S. Provisional Application No. 63/032,410, filed on May 29, 2020, U.S. Provisional Application No. 62/991,446, filed on Mar. 18, 2020, U.S. Provisional Application No. 62/972,921, filed on Feb. 11, 2020, and U.S. Provisional Application No. 62/960,421, filed on Jan. 13, 2020. The entire contents of these applications are incorporated herein by reference in their entirety.

### FIELD OF THE INVENTION

The present invention relates to manipulation of liquids within microfluidic devices.

### REFERENCE TO AN ELECTRONIC SEQUENCE LISTING

The instant application contains a Sequence Listing which has been submitted electronically in XML format and is hereby incorporated by reference in its entirety. Said XML copy, created on Sep. 6, 2024, is named LDX-007WOC2_Sequence_Listing.xml and is 3,235 bytes in size.

### BACKGROUND

A cartridge (e.g., a strip) having a microfluidic channel network may be used, e.g., to determine the presence or amount of one or more targets in a sample liquid and/or determine a physiological property of a sample liquid. Such cartridges may be used in conjunction with a reader, which operates the cartridge to, for example, perform fluidic and/or detection functions in the determination of the target or physiological, physiochemical, or other property of the sample.

Manipulation of a sample and/or other liquids within the cartridge is often performed, for example, in order to ensure that the sample contacts, mixes, and/or reacts with reagents which have been deposited within or introduced to the cartridge.

### SUMMARY OF THE INVENTION

In embodiments, the disclosure relates to a method of manipulating a liquid that is disposed within a capillary channel and has a liquid-gas interface, which includes oscillating a gas pressure of the gas of the liquid-gas interface. The oscillations may induce mixing of materials within the liquid. The materials may include, for example, target compounds or other materials present in the liquid as introduced to the capillary channel and/or reagents or other materials contacted by the liquid within the capillary channel.

In any of the embodiments of the method of manipulating a liquid, the capillary channel may include a proximal origin and a distal terminus. The liquid is introduced to the capillary channel via application to the proximal origin. The liquid-gas interface of the liquid may be a distal liquid-gas interface of the liquid disposed between the proximal origin and the distal terminus of the capillary channel with the gas of the distal liquid-gas interface occupying at least the distal terminus of the capillary channel.

In any of the embodiments of the method of manipulating a liquid, the oscillating the gas pressure may be performed by oscillating the gas pressure, peak-to-peak, by a total relative amount (((Pₘₐₓ−Pₘᵢₙ)/P_{avg})×100) of at least about 5%, at least about 10%, at least about 20%, at least about 25%, or at least about 35%, where Pₘₐₓ is the maximum gas pressure during an oscillation cycle, Pₘᵢₙ is the minimum gas pressure during an oscillation cycle, and P_{avg} is the average gas pressure during an oscillation cycle. The oscillating the gas pressure of the gas may be performed by oscillating the gas pressure, peak-to-peak, by a total relative amount (((Pₘₐₓ−Pₘᵢₙ)/P_{avg})×100) of about 300% or less, about 200% or less, about 135% or less, about 100% or less, or about 75% or less. The oscillating the gas pressure of the gas may be performed by oscillating the gas pressure, peak-to-peak, by a total amount (Pₘₐₓ−Pₘᵢₙ) of at least about 5 kPa, at least about 10 kPa, at least about 20 kPa, at least about 25 kPa, or at least about 35 kPa. The oscillating the gas pressure of the gas may be performed by oscillating the gas pressure, peak-to-peak, by a total amount (Pₘₐₓ−Pₘᵢₙ) of about 200 kPa or less, about 135 kPa or less, about 100 kPa or less, or about 75 kPa or less.

In any of the embodiments of the method of manipulating a liquid, the oscillating the gas pressure may include oscillating a volume occupied by the gas within the capillary channel, e.g., within a distal terminus of the capillary channel. For example, the step of oscillating the gas pressure may be performed by oscillating at least a portion of a wall of the capillary channel over a total distance, peak-to-peak, of at least about 5 μm, at least about 7.5 μm, at least about 15 μm, at least about 20 μm, at least about 25 μm, or at least about 30 μm. The step of oscillating the gas pressure may be performed by oscillating at least a portion of the wall of the capillary channel over a total distance, peak-to-peak, of about 70 μm or less, about 60 μm or less, about 50 μm or less, or about 40 μm or less. The total distance, peak-to-peak may be at least about 5%, at least about 7.5%, at least about 15%, at least about 20%, at least about 25%, or at least about 30% of a total dimension, e.g., height, of the capillary channel along an axis aligned with the oscillatory motion of the wall. The total distance, peak-to-peak may be about 70% or less, about 60% or less, about 50% or less, or about 40% or less of a total dimension, e.g., height, of the capillary channel along an axis aligned with the oscillatory motion of the wall. The distance, and orientation of the dimension of the capillary channel, may be taken along an axis that is generally perpendicular to a longitudinal axis of the capillary channel at the location of oscillation of the wall and/or is generally perpendicular to a plane containing the capillary channel.

In any of the embodiments of the method of manipulating a liquid, the step of oscillating the gas pressure may be performed by oscillating at least a portion of a wall of the capillary channel. The method may include placing the at least a portion of the wall under tension prior to initiating the step of oscillating the at least a portion of a wall. The at least a portion of the wall that is in direct communication with the gas, e.g., directly overlying or underlying the gas, may be not in direct communication with the liquid, e.g., not directly overlying or underlying the liquid. For example, the portion of the wall of the capillary channel that is oscillated and in direct communication with the gas may be spaced apart from the liquid-gas interface, e.g., the distal liquid-gas interface, of the liquid along a longitudinal axis of the capillary by at least about 0.2 cm, at least about 0.3 cm, at least about 0.5 cm, at least about 0.75 cm, at least about 1.00 cm, at least about 1.25 cm, at least about 1.5 cm. The oscillating the at least a portion of a wall of the capillary channel may be performed by subjecting the wall of the capillary channel, e.g., a wall of the distal terminus of the capillary channel, to repeated cycles of deformation and relaxation. The volume occupied by the gas may decrease with increasing deformation and increase with increasing relaxation of the wall of the capillary channel. In an undeformed state, an outer surface of the wall may be generally planar. In a deformed state, an outer surface of the wall may be concave, and become more concave with increasing deformation and an inner surface of the wall may be convex, and become more convex with increasing deformation. Increasing the deformation of the wall may increase tension experienced by the wall and decreasing the deformation of the wall may decrease tension experienced by the wall.

In any of the embodiments of the method of manipulating a liquid, except for the passage of gas along the capillary channel toward and away from the liquid-gas interface, the capillary channel may seal the gas in oscillation with respect to with respect to the ingress and egress of gas thereinto and thereout. For example, the gas in oscillation may occupy a distal terminus of the capillary channel and the distal terminus of the capillary channel may be sealed with respect to the ingress and egress of gas thereinto and thereout except for the passage of gas along the capillary channel toward and away from the liquid-gas interface.

In any of the embodiments of the method of manipulating a liquid, the liquid-gas interface may be a first liquid-gas interface and the liquid that is disposed within a capillary channel may have a plurality of second liquid-gas interfaces. A first plurality of the secondary liquid-gas interfaces may be disposed along a first side wall of the capillary channel. A second plurality of the secondary liquid-gas interfaces may be disposed along a second side wall of the capillary channel, which second side wall may oppose the first side wall. The first liquid-gas interface may have an axis of symmetry generally aligned with a longitudinal axis of the capillary at the location of the first liquid-gas interface and each of the second liquid-gas interfaces may have an axis of symmetry at a non-zero angle with respect to the axis of symmetry of the first liquid-gas interface and/or to the longitudinal axis of the capillary at the location of such secondary liquid-gas interface. The non-zero angle may be at least about 20°, at least about 35°, at least about 45°, at least about 67.5°, or at least about 85°. The non-zero angle may be about 160° or less, about 145° or less, about 112.5° or less, or about 95° or less. For example, the axes of symmetry of the first and secondary liquid-gas interfaces may be generally perpendicular to one another. Alternatively, an axis of symmetry of each of a first set of the secondary liquid-gas interfaces may be oriented at a first angle with respect to the longitudinal axis of the capillary channel and an axis of symmetry of each of a second set of secondary liquid gas interfaces may be oriented at a second different angle with respect to the longitudinal axis of the capillary channel. The first and second angles may oppose one another. For example, the axis of symmetry of each of the first set of secondary liquid gas interfaces may be oriented generally proximally along the capillary channel and the axis of symmetry of each of a second set of liquid gas interfaces may be oriented generally distally along the capillary channel.

In any of the embodiments of the method of manipulating a liquid, the capillary channel may include one or more openings disposed along a first side wall thereof, with the liquid in contact with a gas at each of the one or more openings and forming a secondary liquid-gas interface thereat, e.g., adjacent the first side wall of the capillary channel. The capillary may include one or more openings disposed along a second side wall thereof, with the liquid in contact with a gas at each of the one or more openings in the second side wall and forming a secondary liquid-gas interface thereat, e.g., adjacent the second side wall of the capillary channel. The first and second side walls may oppose one another. Each of the openings in the first and/or second side walls may be an opening to a cavity containing the gas of the secondary liquid-gas interface. Each of the one or more cavities may have a longitudinal axis having an angle of at least about 20°, at least about 35°, at least about 45°, at least about 67.5°, or at least about 85° with respect to the longitudinal axis of the capillary channel at the location of the opening of the cavity to the capillary channel. Each of one or more cavities of the capillary channel may have a longitudinal axis having an angle of about 160° or less, about 145° or less, about 112.5° or less, or about 95° or less with respect to the longitudinal axis of the capillary channel at the location of the opening of the cavity to the capillary channel. For example, the longitudinal axes of each of a plurality of the cavities and the longitudinal axis of the capillary channel at the location of the opening of such cavity to the capillary channel may be generally perpendicular to one another. In embodiments, a longitudinal axis of each of a first set of cavities is oriented at a first angle with respect to the longitudinal axis of the capillary channel and a longitudinal axis of each of a second set of cavities is oriented at a second angle with respect to the longitudinal axis of the capillary channel, where the first and second angles oppose one another. For example, openings of each of a first set of cavities may face generally proximally within the capillary channel and openings of each of a second set of cavities may face generally distally within the capillary channel.

In any of the embodiments of the method of manipulating a liquid including secondary liquid gas interfaces, the secondary liquid gas interfaces may be arranged and configured such that the net effect of oscillating the gas pressure of the gas of the first liquid-gas interface, e.g., oscillating the gas pressure at acoustic frequencies, induces little to no net force, e.g., essentially no net force, tending to induce bulk motion of the first liquid-gas interface along a longitudinal axis of the capillary channel.

In any of the embodiments of the method of manipulating a liquid, the oscillating may be performed at acoustic frequencies, e.g., about 15,000 Hz or less, about 10,000 Hz, e.g., about 5,000 Hz or less, about 3000 Hz or less, about 2000 Hz or less, about 1750 Hz or less, about 1500 Hz or less, about 1250 Hz or less, about 1150 Hz or less, about 1050 Hz or less, or about 950 Hz or less. The oscillating may be performed at about 25 Hz or more, about 50 Hz or more, about 100 Hz or more, about 150 Hz or more, about 200 Hz or more, about 250 Hz or more, about 500 Hz or more, about 750 Hz or more, or about 900 Hz or more.

In any of the embodiments of the method of manipulating a liquid, the oscillating may be performed during a time period T_{osc}. In embodiments, T_{osc} is at least about 1 second, at least about 2 seconds, at least about 5 seconds, at least about 15 seconds, or at least about 20 seconds. In embodiments, T_{osc} of about 180 seconds, about 120 seconds or less, about 90 seconds or less, about 45 seconds or less, or about 30 seconds or less.

The oscillating may be performed at essentially a constant frequency during time T_{osc}. The oscillating may be performed at a frequency that varies during time T_{osc}, for example by increasing or decreasing the oscillation frequency as a linear or non-linear ramp and/or by periodically varying the oscillation frequency, e.g., as a sinusoid, triangle wave, or square wave during time T_{osc}. The oscillation frequency may be varied over a total range of at least about 2.5%, at least about 5%, at least about 7.5%, or at least about 10% of the average frequency during T_{osc}. The oscillation frequency may be varied over a range of about 30% or less, about 25% or less, about 20% or less, or about 15% or less of the average frequency during T_{osc}. The frequency variation may be smooth, or in steps, e.g., steps of about 2.5 Hz, about 5 Hz, about 7.5 Hz, or about 10 Hz. The time to vary the oscillation frequency through the full range of frequency variation, e.g., the period of a periodically variation in time T_{osc}, may be at least about 1%, at least about 2%, at least about 2.5%, at least about 3.5%, or at at least about 5% of time T_{osc}. The time to vary the oscillation frequency through the full range of frequency variation may be at least about 10% or less, about 15% or less, about 10% or less, about 7.5% or less, or about 5% or less, of time T_{osc}. For example, the average oscillation frequency during a T_{osc} of about 25 seconds may be about 1100 Hz and the oscillation frequency may be varied as a triangle wave between about 1050 Hz and about 1100 Hz during T_{osc}, with the triangle wave having a period of about 2 seconds.

In combination, or as an alternative, the oscillating may be performed with essentially a constant peak-to-peak displacement during time T_{osc}. The oscillating may be performed with a peak-to-peak displacement that varies during the oscillating, for example by increasing or decreasing the peak-to-peak displacement as a linear or non-linear ramp during time T_{osc} and/or by periodically varying the oscillation peak-to-peak, e.g., as a sinusoid, triangle wave, or square wave during time T_{osc}.

In any of the embodiments of the method of manipulating a liquid, the oscillation may be performed by oscillating at least a portion of a wall of the capillary at a frequency that is at or substantially the same as a resonance frequency ωr of the wall of the capillary channel. The resonance frequency ωr of the wall may vary as, e.g., a function of the tension of the wall of the capillary channel and/or the composition and structure of the wall. For example, the oscillation frequency may increase with increasing tension of the wall and decrease with decreasing tension of the wall. The resonance frequency ωr of the wall may be determined by using an actuator, such as a piezoelectric actuator, e.g., a piezoelectric bender, to oscillate the wall at a frequency ω1 and then ceasing to drive the oscillation of the wall at the frequency ω1. Once the wall is no longer being driven by the actuator, the wall, which is under tension, continues to move with the magnitude of such movement related to the related to the efficiency of the oscillations driven by the actuator at frequency ω1. The magnitude of motion can be determined, for example, by use of a displacement transducer which converts the movement of the wall to an electrical signal. The displacement transducer may be the actuator used to oscillate the wall at frequency ω1, the mode of operation of which is reversed from that of the actuator to that of a displacement transducer. Upon determining the magnitude of the motion of the wall in response to the wall having been oscillated at frequency ω1, the system again uses the actuator to oscillate the wall, now at a different frequency ω2. For example, the system may reverse the operation of the displacement transducer to again act as an actuator. The system then repeats the steps of ceasing to drive the oscillation of the wall, determining the magnitude of oscillation, and oscillating the wall at a different frequency. The determined magnitude is greatest when the oscillation frequency corresponds to the resonance frequency ωr. Once the resonance frequency ωr is determined, the system continues to drive oscillations of the wall at resonance frequency ωr or a frequency substantially similar thereto. To ensure that the oscillations remain at or near frequency ωr, the system may, after driving the oscillation for a number of cycles at frequency ωr or a frequency near thereto, perform the steps of ceasing to drive the oscillation of the wall at frequency ωr, determining the magnitude of oscillation, and oscillating the wall at a different frequency ωr′, where ωr′ is a frequency near (e.g., without about 3% to 10%) of frequency ωr. Depending on whether the determined magnitude of wall oscillation is greater or smaller than the oscillation at frequency ωr, the system may continue the steps of ceasing to drive the oscillation of the wall, determining the magnitude of oscillation, and oscillating the wall at a different frequency to maintain the oscillation at a frequency of or about the same as the resonance frequency of the wall. For example, the steps of ceasing, determining, and then driving the oscillation of the wall may be repeated at least once within every Nth oscillation wherein N is about 500 or less, about 250 or less, about 125 or less, or about 75 or less.

In any of the embodiments of the method of manipulating a liquid, a location of the liquid-gas interface with respect to a longitudinal axis of the capillary may remain substantially unchanged following a number N oscillations, where N may be, e.g., at least about 500, at least about 1000, at least about 2000, or at least about 3000. The location of the liquid-gas interface with respect to the longitudinal axis of the capillary may remain substantially unchanged following a number N oscillations, wherein N may be, e.g., about 20,000 or less, about 15,000 or less, about 10,000 or less or about 5,000 or less. Following the number N oscillations, the location of the liquid-gas interface may be within, e.g., about 2 mm or less, about 1 mm or less, or about 750 μm or less of its initial location along the longitudinal axis of the capillary channel.

In any of the embodiments of the method of manipulating a liquid including cavities, the opening of each of the one or more cavities may be essentially the only, or the only, route of ingress/egress for gas into or out of the cavity. If the opening is essentially the only route of ingress/egress for gas into or out of the cavity, the other route(s), in total, are insufficient to prevent the formation of a secondary liquid-gas interface adjacent the side wall of the capillary channel. The oscillation may be performed at a frequency that is at or about the same as a resonance frequency of a wall of the capillary channel, which resonance frequency may vary as, e.g., a function of the tension of the wall of the capillary channel and/or the composition and structure of the wall.

In any of the embodiments of the method of manipulating a liquid, a portion of the capillary channel may have a length L along a longitudinal axis of the capillary channel. In any of the embodiments including cavities, a ratio of a total volume of the cavities disposed along the capillary channel portion having the length L to a total volume of the capillary channel, excluding the cavities, along the length L may be at least about 0.03, at least about 0.05, at least about 0.075, at least about 0.085, at least about 0.1, at least about 0.125, or at least about 0.15. A ratio of a total volume of the cavities disposed along the capillary channel portion having the length L to a total volume of the capillary channel, excluding the cavities, along the length L may be about 0.4 or less, about 0.3 or less, about 0.25 or less, about 0.225 or less, or about 0.2 or less. A ratio of a total area of the openings of the cavities disposed along the capillary channel portion having the length L to a total area of an inner surface of the capillary channel, excluding the areas occupied by the cavity openings, along the length L may be at least about 0.0075, at least about 0.009, at least about 0.011, at least about 0.012, or at least about 0.013. A ratio of a total area of the openings of the cavities disposed along the capillary channel portion having the length L to a total area of an inner surface of the capillary channel, excluding the areas occupied by the cavity openings, along the length L may be about 0.05 or less, about 0.04 or less, about 0.03 or less, about 0.02 or less, about 0.0175 or less, or about 0.015 or less.

In any of the embodiments of the method of manipulating a liquid, the manipulation may further include inducing bulk motion of the liquid along a longitudinal axis of the capillary channel sequentially with and/or while simultaneously oscillating the pressure of the gas. For example, the liquid-gas interface may be moved, e.g., by inducing bulk motion of the liquid along the capillary channel, during a total time Tₘₒᵥ from a first position within the capillary channel to a second position separated from the first position by a distance D along a longitudinal axis of the capillary channel. The first position may be distal to or proximal to the second position along the longitudinal axis of the capillary channel. The period Tₘₒᵥ may be, e.g., at least about 1 seconds, at least about 2 seconds, at least about 3 seconds, or at least about 4 seconds. The period Tₘₒᵥ may be, e.g., about 12.5 seconds or less, about 10 seconds or less, or about 7.5 seconds or less. The step of moving the liquid-gas interface may be performed by increasing or decreasing the gas pressure of the gas adjacent the liquid during the period Tₘₒᵥ. As the gas pressure is increased, bulk motion of the liquid is induced in a first direction along the longitudinal axis of the capillary channel and as the gas pressure is decreased, bulk motion of the liquid is induced in a second, opposite direction. Movement of the liquid in response to the changing gas pressure tends to counteract the change so that the gas pressure is essentially the same at the end of time Tₘₒᵥ as at the beginning thereof. The step of increasing or decreasing the gas pressure may be performed by increasing or decreasing a compression of the wall of the capillary channel. For example, the increasing or decreasing the compression may respectively decrease or increase an internal width of the capillary channel along an axis generally perpendicular to the longitudinal axis thereof by a total amount of at least about 7.5 μm, at least about 12.5 μm, at least about 17.5 μm, or at least about 22.5 μm at the end of time Tₘₒᵥ as compared to such width at the beginning thereof. The step of oscillating the gas pressure may be performed during at least a portion, substantially all, essentially all, or during the entire time Tₘₒᵥ.

In any of the embodiments of the method of manipulating a liquid, the length L of the portion of the capillary channel may be, for example, at least about 0.5 mm, 1 mm, at least about 2 mm, at least about 3 mm, or at least about 4 mm. The length L may be, for example, about 25 mm or less, about 17.5 mm or less, about 10 mm or less, about 7.5 mm or less, about 6 mm or less, or about 5 mm or less. The length L may be a multiple N of a distance along the longitudinal axis of the capillary channel between a proximal wall of a first cavity to a proximal wall of an adjacent, distally disposed cavity. The multiple N may be, for example, at least 1, at least 2, at least 3, at least 4, at least 5 or at least 6. The multiple N may be, for example, about 25 or less, about 20 or less, about 15 or less, about 12 or less, about 10 or less, about 8 or less, or about 6 or less. The distance D may, independently, have any of the same dimensions as the length L.

In any of the embodiments of the method of manipulating a liquid, the capillary channel may be a microchannel, e.g., an analysis channel, within a microfluidic channel network of a microfluidic device (e.g., microfluidic strip). The wall of the microchannel is a layer, e.g., a substrate, of the microfluidic strip.

In any of the embodiments of the method of manipulating a liquid, the oscillating may be performed by oscillating an actuator in contact with the outer surface of a wall of the capillary channel. The actuator may be a piezoelectric actuator, e.g., a piezoelectric bender.

In embodiments, a method includes introducing a sample liquid to a microchannel of a microfluidic device (e.g., a microfluidic strip), the sample liquid occupying a first portion of the microchannel, a second portion of the microchannel adjacent to the first portion being occupied by a gas, the sample liquid and the gas forming a liquid-gas interface therebetween; and repeatedly imparting energy to the gas in the second portion of the microchannel, wherein at least some of the energy is transferred from the gas to the sample liquid via the liquid-gas interface.

In embodiments, a method of imparting energy to a liquid that is disposed within a capillary channel and has a plurality of liquid-gas interfaces includes imparting energy to the liquid at a frequency substantially similar to a resonance frequency of the liquid with respect to the liquid-gas interfaces. The method may include inducing bulk motion of the liquid along a longitudinal axis of the capillary channel sequentially with and/or while simultaneously imparting energy to the liquid. The capillary channel may include a plurality of openings disposed along a side wall thereof, with the liquid in contact with a gas at each of the one or more openings and one of the plurality of liquid-gas interfaces thereat, e.g., adjacent the side wall of the capillary channel. Each of the plurality of liquid-gas interfaces may have an axis of symmetry at a non-zero angle with respect to the axis of symmetry of the longitudinal axis of the capillary channel at the location of such liquid-gas interface. The non-zero angle may be at least about 20°, at least about 35°, at least about 45°, at least about 67.5°, or at least about 90°. The non-zero angle may be about 160° or less, about 145° or less, about 135° or less, or about 120° or less. For example, the axes of symmetry of the liquid-gas interfaces and the longitudinal axis of the capillary channel may be generally perpendicular to one another. Each of the openings may be an opening to a cavity containing the gas of at least one of the liquid gas interfaces. Each of the one or more cavities may have a longitudinal axis having an angle of at least about 20°, at least about 35°, at least about 45°, at least about 67.5°, or at least about 85° with respect to the longitudinal axis of the capillary channel at the location of the opening of the cavity to the capillary channel. Each of the one or more cavities of the capillary channel may have a longitudinal axis having an angle of about 160° or less, about 145° or less, about 135θ or less, or about 120° or less with respect to the longitudinal axis of the capillary channel at the location of the opening of the cavity to the capillary channel. For example, the longitudinal axes of each of a plurality of the cavities and the longitudinal axis of the capillary channel at the location of the opening of such cavity to the capillary channel may be generally perpendicular to one another.

In any of the embodiments of the method of imparting energy to the liquid and including such cavities, the cavities may be arranged and configured such that the net effect of imparting energy induces little to no force tending to propel the liquid along the longitudinal axis of the capillary channel. For example, when imparting energy the net effect of a plurality of side cavities arranged within a reagent or detection zone of the capillary channel may induce insufficient force to propel the liquid out of such reagent or detection zone during a time period sufficient to mobilize a dried reagent present therein, mix a sample liquid and a reagent disposed therein, and/or incubate the reaction between a target and a reagent disposed therein. In embodiments, a longitudinal axis of each of a first set of cavities is oriented at a first angle with respect to the longitudinal axis of the capillary channel and a longitudinal axis of each of a second set of cavities is oriented at a second angle with respect to the longitudinal axis of the capillary channel, where the first and second angle oppose one another. For example, openings of each of the first set of cavities may face generally proximally within the capillary channel and openings of each of the second set of cavities may face generally distally within the capillary channel. Alternatively, or in combination, the longitudinal axes of each of a plurality of cavities and the longitudinal axis of the capillary channel at the location of such cavity within a capillary channel, e.g., within a reagent or detection zone of the capillary channel, may be generally perpendicular to one another.

In any of the embodiments of the method of imparting energy to the liquid and including such cavities, the opening of each of the one or more cavities may be essentially the only, or the only, route of ingress/egress for gas into or out of the cavity. If the opening is essentially the only route of ingress/egress for gas into or out of the cavity, the other route(s), in total, are insufficient to prevent the formation of a secondary liquid-gas interface adjacent the side wall of the capillary channel. The oscillation may be performed at a frequency that is at or about the same as a resonance frequency of the wall of the capillary channel, which resonance frequency may vary as, e.g., a function of the tension of the wall of the capillary channel and/or the composition and structure of the wall.

In any of the embodiments of the method of imparting energy to the liquid the imparting energy may be performed by repeatedly modifying (e.g., by increasing and decreasing) a pressure of a gas adjacent a liquid-gas interface of the liquid. The step of repeatedly increasing and decreasing a pressure of the gas may be performed by oscillating a wall of the microchannel wherein the wall is in direct communication with the gas, e.g., directly overlying or underlying the gas, and not in direct communication with the liquid, e.g., not directly overlying or underlying the liquid. For example, the portion of the wall that is oscillated may be spaced apart from the liquid-gas interface of the liquid along a longitudinal axis of the capillary by at least about 0.2 cm, at least about 0.3 cm, at least about 0.5 cm, at least about 0.75 cm, at least about 1.00 cm, at least about 1.25 cm, at least about 1.5 cm.

In any of the embodiments of the method of imparting energy to the liquid, the imparting energy may be performed at acoustic frequencies, e.g., about 15,000 Hz or less, about 10,000 Hz, e.g., about 5,000 Hz or less, about 3000 Hz or less, about 2000 Hz or less, about 1750 Hz or less, about 1500 Hz or less, about 1250 Hz or less, about 1150 Hz or less, about 1050 Hz or less, or about 950 Hz or less. The oscillating may be performed at about 25 Hz or more, about 50 Hz or more, about 100 Hz or more, about 150 Hz or more, about 200 Hz or more, about 250 Hz or more, about 500 Hz or more, about 750 Hz or more, or about 900 Hz or more.

In embodiments, a microfluidic device (e.g., a microfluidic strip) includes a microfluidic channel network and first and second electrically conductive leads. A first portion of each electrically conductive lead is disposed within a respective different, liquid sensing location of the microfluidic channel network. Each liquid sensing location is a location of the microfluidic device occupied by a liquid during use of the microfluidic device. A second portion of each electrically conductive lead is disposed at a different, mechanical sensing location of the microfluidic device. Each mechanical sensing location is a location of the microfluidic device at which a mechanical manipulation and/or operation of and/or upon the microfluidic device modifies an electrical property of the respective electrically conductive lead. In some embodiments, the microfluidic device includes an electrically conductive bridging member configured to modify an electrical property of at least one (e.g., both) of the respective second portion(s) of the first and second electrically conductive leads upon the mechanical manipulation and/or operation of or upon the microfluidic device. For example, the electrically conductive bridging member may increase or decrease an impedance or resistance between the first and second portions upon the mechanical manipulation and/or operation of or upon the microfluidic device. At least one (e.g., both) of the respective mechanical sensing locations may be a location configured to be maintained in a dry state, e.g., not occupied by liquid, during use of the microfluidic device.

In embodiments, a method of using a microfluidic device (e.g., a microfluidic strip) includes (i) mechanically modifying a shape and/or configuration of the microfluidic device and sensing the occurrence and/or extent of the mechanically modifying by detecting a first electrical signal at at least one of a first electrical contact and a second electrical contact of the microfluidic device, (ii) sensing the presence of a liquid and/or performing at least one electrochemical determination, e.g., of the presence and/or amount of a second target, at at least one first location within a microfluidic channel network of the microfluidic device by detecting a second electrical signal at at least the first electrical contact, and (iii) sensing the presence of a liquid and/or performing at least one electrochemical determination, e.g., of the presence and/or amount of a second target, at at least one second location within the microfluidic channel network of the microfluidic device by detecting a third electrical signal at at least the second electrical contact, where the at least one second location(s) is spaced apart from the at least one first location(s) within the microfluidic channel network of the microfluidic device. In some embodiments, the third electrical signal arises from a change in impedance, e.g., a change in continuity, between respective portions of first and second electrically conductive leads, each first and second electrically conductive leads being in electrical communication with a respective one of the first and second electrical contacts. The sensing of the presence of liquid at the at least one first location may include sensing an electrical signal arising from a first electrode in contact with the sample liquid at the at least one first location, the first electrode being in electrical communication with the first electrically conductive lead and the first contact. The sensing of the presence of liquid at the at least one second location may include sensing an electrical signal arising from a second electrode in contact with the sample liquid at the at least one second location, the second electrode being in electrical communication with the second electrically conductive lead and the second contact.

In embodiments, a method of modifying a volume of a gas bladder of a microfluidic device (e.g., a microfluidic strip), includes providing a microfluidic device including a microfluidic channel network, a gas bladder in gaseous communication with the microfluidic channel network, and a gas bladder sensor in sensing communication with the gas bladder. Using an actuator, the volume of the gas bladder can be modified (e.g., decreased) to expel gas from the gas bladder into the microfluidic channel network and/or modified (e.g., increased) to withdraw gas from the microfluidic channel network into the gas bladder. The expelling of gas from the gas bladder moves liquid present within the microfluidic channel network in a first direction therein and the withdrawal of gas into the gas bladder moves such liquid in a second different (e.g., opposite) direction therein. The method includes using the actuator to modify the volume of the gas bladder to a first extent (e.g., to decrease and/or increase the volume), sensing at least one gas bladder signal from the gas bladder sensor indicative of the first extent of volume modification and at least one actuator signal indicative of the extent of actuation that corresponds to the first extent of volume modification, and storing at least the actuator signal(s) or a signal(s) indicative thereof. After the step of modifying the volume to the first extent, the method includes moving liquid within the microfluidic channel at least one time by using the actuator to further modify the volume of the gas bladder (e.g., to decrease and/or increase the volume). After the step of moving the liquid, the method includes using the actuator to modify the volume of the gas bladder to a second extent having a predetermined relationship to the first extent as determined from the stored actuator signal(s) or signal(s) indicative thereof.

In any embodiment of the method of modifying a volume of a gas bladder of a microfluidic device, the first extent of volume modification may correspond to an operationally fully compressed state of the gas bladder. The second extent of volume modification may be substantially the same, e.g., essentially the same as the first extent of volume modification.

In any embodiment of the method of modifying a volume of a gas bladder of a microfluidic device, the gas bladder sensor includes any of the embodiments of first and second electrically conductive leads. For example, the gas bladder sensor may include first and second electrode leads and a bridging contact configured to bring the first and second leads into electrical communication when the gas bladder volume is modified to the first extent. The first and second electrode leads may each be in electrical communication with a respective electrode configured to sense the presence of liquid within the microfluidic channel network.

In any embodiment of the method of modifying a volume of a gas bladder of a microfluidic device, the actuator is an actuator of a reader configured to operate the microfluidic device to the presence or amount of one or more targets in a sample liquid and/or determine a physiological property of a sample liquid. The actuator may be a piezoelectrically driven actuator. The actuator may compress an external wall of the gas bladder to reduce a volume thereof.

In embodiments, a microfluidic channel network includes first and second electrodes, each having at least a respective portion disposed within the microfluidic channel network in a respective different location to contact liquid present in the microfluidic channel network. Liquid, e.g., a sample liquid or a reagent liquid such as a buffer, disposed within the microfluidic channel network and connecting, e.g., extending between, the first and second electrodes reduces the impedance or resistance between the first and second electrodes as compared to, e.g., a gas such as air. Accordingly, an electrical signal applied at the first electrode may be detected at the second electrode in the presence of the liquid. However, if one or more portions of the microfluidic channel network disposed between the first and second electrodes are not fully occupied by sample liquid, e.g., are occupied by a gas such as air, the electrical signal is not detected at the second electrode.

In any of the embodiments of the microfluidic channel network including first and second electrodes, the microfluidic channel network may include multiple interconnected microchannels. The first and second electrodes may be disposed within the same or within different microchannels of the microchannel network. In some embodiments, the microfluidic channel network includes a single microchannel. In some embodiments, a shortest distance between the first and second electrodes along the one or more microchannels of the microchannel network is at least about 1 cm, at least about 1.5 cm, at least about 2 cm, or at least about 2.5 cm. In some embodiments, the microfluidic channel network includes one or more additional second electrodes at which the electrical signal can be detected in the presence of the sample liquid, each such additional second electrode disposed at a different location within the microfluidic channel network.

In any of the embodiments of the microfluidic channel network including first and second electrodes, the microfluidic channel network may be formed within a microfluidic device (e.g., a microfluidic strip). The electrodes may be connected to a portion of the strip remote from the microchannel network, e.g., to a periphery of the strip, via electrically conductive leads by which the electrical signal can be introduced to the first electrode and detected at the second and one or more additional electrodes.

In embodiments, a method of using any of the embodiments of the microfluidic channel network including first and second electrodes includes generating an electrical signal at the first electrode and determining whether the electrical signal is present at the second electrode. The electrical signal may be a time varying signal such as a sine wave, square wave, or triangle wave. The time varying signal may have a DC offset, which may be of sufficient magnitude that the time varying signal is substantially, e.g., essentially or entirely of a single polarity, e.g., positive or negative, with respect to ground.

In embodiments, a method includes providing a microfluidic device that has a microchannel network including first and second electrodes and two or more channels, e.g., analysis channels. The first electrode is in electrical communication with a first location within the microchannel network that is spaced apart from each of the two or more channels. The second electrode is in electrical communication with the microchannel network at a second location that is spaced apart from the first location and from the two or more channels, at a third location disposed within the first channel, and at a fourth location disposed within the second channel. Sample liquid applied to the strip establishes continuity between the first electrode and the second electrode along each of three pathways within the microchannel network: (1) between the first and second locations along a pathway that excludes the first and second channels, (2) between the first location and the third location within the first channel, and (3) between the first location and the fourth location within the second channel. A time varying signal may be applied to the first electrode at the first location, e.g., by applying the time varying signal to a contact of the first electrode which may be positioned at or near a periphery of the strip. The time varying signal received by the second electrode at the second, third, and/or fourth electrodes may be measured, e.g., at a contact of the second electrode which may be positioned at or near a periphery of the strip. Based on the signal received, a reader can determine whether liquid has filled the microchannel network between the first location and the second, third, and/or fourth locations or combination thereof.

In embodiments, a method of moving a liquid includes moving the liquid in a first direction along a capillary channel and detecting a first electrical signal indicative of the liquid having come into contact with a first electrode disposed within the capillary channel. After detecting the first electrode signal, the method includes ceasing the moving of the liquid and thereafter moving the liquid in a second, opposite direction along the capillary channel. Upon or after initiating the moving of the liquid in the second direction, the method may include detecting a cessation of the first electrode signal indicating that the liquid has moved away from, e.g., is no longer in contact with the first electrode. The method may include detecting a second electrical signal indicative of the liquid being in contact with a second electrode disposed within the capillary channel and spaced apart from the first electrode in the second direction. The detecting the second electrical signal may be performed during at least a portion of the time of performing the step of moving the liquid in the second direction. The method may include detecting a cessation of the first electrode signal indicating the that liquid has moved away from e.g., is no longer in contact with the second electrode. After detecting the cessation of the second electrode signal, the method may include ceasing the moving of the liquid in the second direction.

In any of the embodiments of the method of moving a liquid, the first electrical signal indicative of the liquid having come into contact with the first electrode disposed within the capillary channel may be indicative of a liquid-gas interface of the liquid having displaced a gas from the location of the first electrode as the liquid moves in the first direction. The cessation of the first electrical signal may be indicative of the gas again occupying the location of the first electrode as the liquid-gas interface moves in the second direction. The cessation of the second electrical signal may be indicative of the gas occupying the location of the second electrode with the liquid-gas interface of the liquid having moved past the second electrode proceeding in the second direction away from the first electrode.

In any of the embodiments of the method of moving a liquid, after ceasing the moving of the liquid in the second direction, the method includes repeating the steps of moving the liquid in the first direction, detecting the first electrical signal, and ceasing moving the liquid in the first direction. After repeating the step of ceasing moving the liquid in the first direction, the method may include repeating the steps of moving the liquid in the second, opposite direction, detecting the second electrical signal, detecting the cessation of the second electrical signal and ceasing the moving of the liquid in the second direction. The sequence of steps may be repeated a number N times, where N is at least 2, at least about 5, at least about 10, at least about 20, or at least about 25.

In any of the embodiments of the method of moving a liquid, the first and second electrodes are spaced apart by a distance D along the capillary channel where D is, for example, at least about 0.5 mm, 1 mm, at least about 2 mm, at least about 3 mm, or at least about 4 mm. The distance D may be, for example, about 25 mm or less, about 17.5 mm or less, about 10 mm or less, about 7.5 mm or less, about 6 mm or less, or about 5 mm or less. The moving of the liquid in the first or second direction may be performed at a velocity of at least about 0.2 mm s⁻¹, at least about 0.5 mm s⁻¹, at least about 0.75 mm s⁻¹, or at least about 1.0 mm s⁻¹. The moving of the liquid in the first or second direction may be performed at a velocity of about 4 mm s⁻¹ or less, about 3 mm s⁻¹ or less, about 2 mm s⁻¹ or less, or about 1.5 mm s⁻¹ or less.

In any of the embodiments of the method of moving a liquid, one or both of the first and second electrodes is disposed adjacent to at least a first, or at least a first and a second, hydrophobic layer disposed within the capillary channel. Each of the hydrophobic layer(s) may cover a first portion of the electrode within the capillary channel. For example, the first and second hydrophobic layers may cover respective first portions of the electrode. The covered first portions of the electrode may be disposed adjacent opposed side walls of the capillary channel leaving a second, uncovered portion of the electrode disposed in a central portion of the capillary channel along an axis transverse to a longitudinal axis of the capillary channel.

In any of the embodiments of the method of moving a liquid, the method includes oscillating a gas pressure of the gas of the liquid-gas interface while moving the liquid in the first and/or second direction. The liquid-gas interface may be a first liquid-gas interface having an axis of symmetry generally aligned with a longitudinal axis of the capillary. The capillary channel may include one or more openings disposed along a side wall thereof, with the liquid in contact with a gas at each of the one or more openings and forming a secondary liquid-gas interface thereat. In any of the embodiments of the method of moving a liquid, each of the one or more secondary liquid-gas interfaces may have an axis of symmetry generally perpendicular to the axis of symmetry of the first liquid-gas interface and to the longitudinal axis of the capillary. The oscillation may be performed at a frequency that is at or about the same as a resonance frequency of the liquid in the capillary channel in communication with the secondary liquid-gas interfaces.

In any of the embodiments of the method of moving a liquid, each of the one or more openings disposed in the side wall may an opening to a cavity containing the gas of the secondary liquid-gas interface. The opening of each of the one or more cavities may be the only route of ingress/egress for gas into or out of the cavity.

In any of the embodiments of the method of moving a liquid, the capillary channel may be a capillary channel within a microfluidic channel network of a microfluidic strip. The first and second electrodes may be connected to a portion of the strip remote from the microchannel network, e.g., to a periphery of the strip, via electrically conductive leads by which the first and second electrical signals can be detected.

In embodiments, a microfluidic device (e.g., a microfluidic strip) includes a reagent. The microfluidic device may include first and second generally planar layers, e.g., substrates, each having a respective, opposed surface. The respective opposed surfaces of the first and second layers are spaced apart by at least one third layer that secures, e.g., adheres, the first and second layers in opposition. The at least one third layer occupies less than all of the area between the first and second layers with a microfluidic channel network being defined at least in part by the unoccupied portions of the area between the first and second layers. Internal opposed surfaces of the first and second layers unoccupied by the third layer define respective upper and lower internal surfaces of the microchannel network and respective internal surfaces of the third layer abutting the unoccupied portions of the area between the first and second layers define side walls of the microchannel network. A reagent is disposed on the opposed surface of at least one of the first and second layers within a channel of the microchannel network. At least a first portion of the reagent is disposed within the channel on a portion of such opposed surface unoccupied by at least a portion of the at least one third layer. At least a second portion of the reagent is disposed outside of the channel on a portion of such opposed surface occupied by the at least one third layer. The third layer overlies the second portion of the reagent. The second portion of the reagent may be disposed adjacent, e.g., abutting, the first portion of reagent outside of a first side wall of the channel. At least a third portion of the reagent may be disposed outside of the channel on a portion of such opposed surface occupied by the at least one third layer outside of a second side wall of the channel of the microchannel network, wherein the second side wall is opposed to the first side wall across the channel. The third layer overlies the second portion of the reagent.

In any of the embodiments of the microfluidic device including a reagent, the third layer may define a plurality of cavities adjacent the microchannel network. The capillary channel may include one or more openings disposed along a side wall thereof, with the liquid in contact with a gas at each of the one or more openings and forming a secondary liquid-gas interface thereat. Each of the one or more secondary liquid-gas interfaces may have an axis of symmetry generally perpendicular to the axis of symmetry of the finrst liquid-gas interface and to the longitudinal axis of the capillary.

In embodiments, a method of manufacturing a microfluidic device, e.g., a microfluidic strip, includes providing first and second layers, e.g., substrates; depositing a reagent on a portion, but not all, of a first surface of the first layer; disposing a first surface of at least one third layer on the first surface of the first layer; and disposing a first surface of the second layer on a second surface of the at least one third layer; wherein (i) the at least one third layer (a) occupies less than all of the area of the first surface of the first layer and the first surface of the second layer and (b) secures the first and second layers in opposition with respect to one another with at least a first portion of the third layer overlying some, but not all, of the deposited reagent; (ii) at least a portion of the first surface of the first layer not occupied by the third layer, at least a portion of the first surface of the second layer not occupied by the third layer define first and second inner surfaces of a microfluidic channel network with at least a portion of the deposited reagent disposed on the first surface of the first layer within the microfluidic channel network.

In any of the embodiments of the method of manufacturing a microfluidic device, the method may include, prior to the step of depositing the reagent, depositing a reagent deposition boundary on the first surface of the first substrate. The reagent deposition boundary limits the extent of the area occupied by the reagent upon deposition on the first surface of the first substrate. The reagent deposition boundary may be formed of a hydrophobic layer or film, e.g., an ink. At least a portion, e.g., a majority, essentially all, or all of the reagent deposition boundary may be deposited in a portion of the first surface of the first layer to be overlaid by the third layer.

In any of the embodiments of the method of manufacturing a microfluidic device, the method may include providing side cavities within edges of the third layer adjacent portions of the first surfaces of the first and second layers not occupied by the third layer and on which the reagent is deposited. In use, each cavity forms a liquid-gas interface with liquid present in the microfluidic channel network.

In any of the embodiments of the method of manufacturing a microfluidic device, the microfluidic strip (e.g., device) may be configured to perform an assay to determine the presence and/or amount of at least one target present in a liquid applied to the microfluidic device.

In embodiments, a microfluidic device (e.g., a microfluidic strip) includes a microfluidic channel network having a sample application zone, a common branch channel in fluidic communication with the sample application zone, and a plurality of analysis channels, each having a proximal origin connected to the common branch channel at a first location therealong and a distal terminus spaced apart from the proximal origin by such analysis channel. Each of the first locations may be different from the other first locations. The microfluidic channel network includes a vent in gaseous communication with the common branch channel. For each of the plurality of analysis channels, the proximal origin provides the only route by which liquid and gas may enter or exit such analysis channel. Each analysis channel includes a gas bladder, e.g., adjacent to or defining the distal terminus thereof. Compressing the gas bladder of an analysis channel decreases the volume of the gas bladder and expels gas from the gas bladder toward the proximal origin of such analysis channel. A sample liquid, if present in the analysis channel, is moved along the analysis channel away from the gas bladder toward the proximal origin of such analysis channel. Decompressing the gas bladder of an analysis channel increases the volume of the gas bladder and draws gas from the analysis channel into such gas bladder. A sample liquid, if present in the analysis channel, is moved along the analysis channel toward the decompressed gas bladder.

In some embodiments, the vent and the sample application zone are the only routes by which gas may enter or exit the microfluidic channel network. In some embodiments, the vent is spaced apart from the common branch channel by at least a vent channel. The vent channel may have a cross sectional area of about 20,000 mm² or less, about 18,000 mm² or less, or about 17,000 mm² or less. The vent channel may have a cross sectional area of at least about 5,000 mm², at least about 10,000 mm², or at least about 12,500 mm². The vent channel may have a length of at least about 7,500 mm, at least about 10,000 mm, at least about 12,500 mm. The vent channel may have a length of about 20,000 mm or less, or about 17,500 mm or less. In some embodiments, each of the analysis channels has a length of at least about 10,000 mm, at least about 15,000 mm, at least about 17,500 mm. The analysis channels may have a length of about 35,000 mm or less, about 30,000 mm or less, or about 27,500 mm or less.

In some embodiments, the analysis channels are first analysis channels and the microfluidic network includes a second analysis channel having a proximal origin connected to the common branch channel at a second location therealong and being gaseously connected to the vent at a distal terminus thereof. For example, the vent channel may include a distal terminus at the vent and a proximal origin connected to the distal terminus of the second analysis channel. The second analysis channel may be configured to determine the hematocrit of a blood sample applied to the sample application zone of the microfluidic device. The second location may be different from each of the first locations.

In some embodiments, the microfluidic device includes a distal portion configured to be received within a reader during operation of the microfluidic device. Each of the gas bladders is located within the distal portion of the microfluidic device. The microfluidic device includes a proximal portion configured to protrude from the reader during operation of the microfluidic device. The sample application zone and the vent are located within the proximal portion of the microfluidic device.

In embodiments, a microfluidic device (e.g., a microfluidic strip) includes a microfluidic channel network having a sample application port and a supply channel extending from the sample application zone. The microfluidic device includes at least one zone of soluble anticoagulant disposed within the sample application port, the supply channel, or combination thereof. The soluble anticoagulant may be in a dry state. The at least one zone of soluble anticoagulant may be disposed (i) within or adjacent the sample application port, or in both locations, or (ii) within the supply channel and spaced apart from the sample application port. If present within the supply channel and spaced apart from the sample application port, the at least one zone of soluble anticoagulant may be spaced apart from the sample application port by a length of the supply channel, e.g., by a length of at least about 3 mm, at least about 5 mm, at least about 7.5 mm, or at least about 10 mm, that is essentially free or free of soluble anticoagulant. The at least one zone of anticoagulant may be a first zone of anticoagulant that is disposed within or adjacent the sample application port and the microfluidic device may include a second zone of soluble anticoagulant (e.g., in a dry state) disposed within the supply channel and spaced apart from the first zone of anticoagulant by a length of the supply channel, e.g., by a length of at least about 3 mm, at least about 5 mm, at least about 7.5 mm, at least about 10 mm, at least about 12.5 mm, or at least about 15 mm that is essentially free or free of soluble anticoagulant. The soluble anticoagulant may comprise or consist essentially of lithium heparin.

In embodiments, a method includes introducing a sample, e.g., a blood based sample, to a sample application port of a microfluidic device and flowing the sample along a microchannel extending from the sample application port within the microfluidic device. The flowing includes contacting the sample with a first zone of anticoagulant disposed within or adjacent the sample application port and a second zone of anticoagulant disposed within the channel and spaced apart from the sample application port and the first zone of anticoagulant by a length of the channel that is essentially free or free of soluble anticoagulant. The length may be, for example at least about 3 mm, at least about 5 mm, at least about 7.5 mm, at least about 10 mm, at least about 12.5 mm, or at least about 15 mm. The soluble anticoagulant may be in a dry state prior to contact with the sample. The soluble anticoagulant may comprise or consist essentially of lithium heparin. The method may further include combining the sample that has contacted the soluble anticoagulant with a reagent within a channel of the microfluidic device and using the reagent to perform a diagnostic assay, e.g., an immunological assay, for the presence of one or more targets in the sample. The one or more targets may be an antigen of a coronavirus such as, for example, SARS-CoV-2.

In embodiments, a microfluidic device (e.g., a microfluidic strip) includes a microfluidic channel network including a plurality of microchannels. One or more of the microchannels include at least a first internal surface. Liquid within one or more microchannels contacts the first internal surface. The internal surface is substantially diffusely reflective within at least one wavelength band. Within the wavelength band, at least 50%, at least 65%, at least 75%, at least 90%, at least 95%, or at least 99% of light that is reflected from incident light striking the surface at an angle of between about 0° and about ±450 with respect to a surface normal when the surface is dry is diffusely reflected rather than directly reflected at the angle of incidence. Within the wavelength band, the diffuse reflection may be substantially uniform, e.g., Lambertian, or may be preferential with lobes or maxima of reflectance in certain directions. The reflectance of the diffusely reflective surface may be at least 90%, at least 92%, at least 95%, or at least 97.5% within a 100 nm wide wavelength band within the range of 400 nm to 2500 nm, or 600 nm to 2200 nm, or 800 nm to 1500 nm.

In embodiments, the diffusely reflective surface includes a metal oxide, such as aluminum oxide, or a crystalline material or mineral such as barium sulfate. The microfluidic device may include a layer, e.g., a polymer layer, and the diffusely reflective internal surface may be a coating or layer applied over at least a portion of the total area of the layer.

With respect to a longitudinal axis of the one or more microchannels, the diffusely reflective internal surface may have a length of at least about 1 mm, at least about 2 mm, at least about 3 mm, or at least about 4 mm and/or a length of about 10 mm or less, about 7.5 mm or less, or about 6 mm or less. In the location of the diffusely reflective internal surface, the microchannel may have a width along an axis normal to the longitudinal axis of at least about 500 μm, at least about 750 μm, or at least about 1000 μm and/or a width of about 2000 μm or less, about 1500 μm or less or about 1250 μm or less. The diffusely reflective internal surface may occupy substantially all of the width and/or area of the internal surface of the channel within the length of the diffusely reflective internal surface.

In some embodiments, a microfluidic device (e.g., a microfluidic strip) is configured to perform a serology immunoassay (e.g., a bridge serology assay) for antibodies to SARS-CoV-2. The microfluidic strip includes a microfluidic channel network including a sample application port and an analysis channel in fluidic communication therewith. The analysis channel includes a first reagent and a second reagent. The first reagent includes a SARS-CoV-2 spike glycoprotein S1 subunit, or fragment thereof, and the second reagent includes a SARS-CoV-2 receptor binding domain (RBD), or fragment thereof. In certain embodiments, the first and second reagents include a SARS-CoV-2 S1 spike glycoprotein. If a fragment of the spike glycoprotein S1 subunit is used, the fragment retains the ability to bind specifically with antibodies to the SARS-CoV-2 spike glycoprotein S1 subunit, as such antibodies may be present in a mammalian (e.g., human) subject as a result of a previous or current infection with SARS-CoV-2. If a fragment of the SARS-CoV-2 RBD is used, the fragment retains the ability to bind specifically with antibodies to the SARS-CoV-2 RBD, as such antibodies may be present in a mammalian, e.g., (human) subject as a result of a previous or current infection with SARS-CoV-2.

In some embodiments, one of the first reagent and the second reagent is bound to, or is configured to bind to a capture agent (e.g., a surface, such as a surface of a channel of the microchannel network, or a particle, such as a magnetic particle) and the other of the first reagent and the second reagent is bound to or is configured to bind to a detectable label. For example, the first reagent may be a conjugate including (i) SARS-CoV-2 S1 spike glycoprotein S1 subunit, or fragment thereof and (ii) a binding agent configured to bind to a capture agent (e.g., a surface, such as a surface of a channel of the microchannel network, or a particle, such as a magnetic particle). For example, the conjugate may include one of biotin and streptavidin and the particle or surface may include the other of biotin and streptavidin, e.g., the first reagent may be a conjugate of SARS-CoV-2 spike glycoprotein S1 subunit, or fragment thereof, and biotin and the microfluidic strip may further include a particle, e.g., a magnetic particle, conjugated to streptavidin. The second reagent may be a conjugate including (i) SARS-CoV-2 RBD, or fragment thereof and (ii) a detectable label such as a fluorescent particle, e.g., a fluorescent latex particle.

In embodiments, a method of performing a serology immunoassay for antibodies to SARS-CoV-2 includes combining a liquid sample, e.g., a blood-based sample, suspected of containing such antibodies with a first reagent including a SARS-CoV-2 spike glycoprotein S1 subunit, or fragment thereof, and a second reagent including SARS-CoV-2 RBD, or fragment thereof and determining the presence and/or amount of a complex including the first reagent, the antibody, and the second reagent. The method may include applying the liquid sample to a sample application zone of a microfluidic device including one or both of the first reagent and the second reagent in a microfluidic channel network of the microfluidic device. The first reagent may be a conjugate including (i) SARS-CoV-2 spike glycoprotein S1 subunit, or fragment thereof and (ii) a binding agent configured to bind to a surface or particle such as a magnetic particle. For example, the first reagent may be a conjugate including (i) SARS-CoV-2 spike glycoprotein S1 subunit, or fragment thereof and (ii) biotin, and the method may further include combining the liquid sample with a third reagent including a conjugate of a magnetic particle and streptavidin. In certain embodiments, the first reagent may be a conjugate including (i) SARS-CoV-2 spike glycoprotein S1 subunit, or fragment thereof and (ii) biotin, which is bound to a conjugate including a magnetic particle and streptavidin prior to introduction of a sample. The second reagent may be a conjugate including (i) SARS-CoV-2 RBD, or fragment thereof and (ii) a detectable label such as a fluorescent particle, e.g., a fluorescent latex particle. In some embodiments, the first, second, and/or third reagents are disposed within an analysis channel of the microfluidic channel network. A distal portion of the analysis channel may include a gas bladder and the method may include compressing, decompressing and/or oscillating the gas bladder as disclosed herein to manipulate the liquid sample e.g., to move the liquid sample and/or mix the liquid sample and reagents as disclosed herein. The method may include magnetically retaining complexes of the third reagent, the first reagent, an antibody to SARS-CoV-2, and the second reagent in a detection zone of the microfluidic channel network prior to detecting the complexes. The method may include expelling sample liquid from the detection zone as disclosed herein prior to the detecting step.

In some embodiments, the microfluidic device (e.g., microfluidic strip) is configured to perform an assay to detect an antigen, e.g., a SARS-CoV-2 antigen, in a sample, e.g., a nasal, nasopharyngeal, or saliva sample. The sample may be from, e.g., blood-based sample such as blood, plasma, or serum, or a nasal or nasopharyngeal swab specimen and/or contained in Universal Transport Media (UTM) or Viral Transport Media (VTM). The sample can comprise blood, serum, or plasma, e.g., wherein the sample comprises or consists essentially of serum and/or plasma. In certain embodiments, the sample may not be subjected to a lysis step (e.g., a lysis step sufficient to lyse white blood cells, red blood cells, or virus, e.g., coronavirus such as SARS-CoV-2, within the sample) prior to the detection assay. In certain embodiments, the step of subjecting the sample to a binding assay is performed without releasing coronavirus antigen from cells present in the sample, e.g., without releasing coronavirus antigen from within white blood cells, red blood cells, or from either of white blood cells or red blood cells. In certain embodiments, the step of subjecting the sample to a binding assay is performed without first contacting the sample with a chemical lysis reagent, e.g., without first contacting the sample with an alkali, detergent, or enzyme in sufficient concentration to rupture the walls of cells, e.g., the walls of white blood cells, red blood cells, or from either of white blood cells or red blood cells present in the sample. In certain embodiments, the step of subjecting the sample to a binding assay is performed without first subjecting the sample to a physical lysis step, e.g., without first subjecting the sample to thermal conditions, osmotic pressure, shear forces, or cavitation sufficient to rupture the walls of cells, e.g., the walls of white blood cells, red blood cells, or from either of white blood cells or red blood cells present in the sample. In certain embodiments, the step of subjecting the sample to a binding assay is performed without first subjecting the sample to a lysis step sufficient to lyse coronavirus in the sample, e.g., without first subjecting the sample to a lysis step sufficient to lyse SARS-CoV-2 present in the sample. In certain embodiments, when coronavirus antigen is detected, substantially all of the detected coronavirus antigen is free antigen, e.g., antigen not associated with intact virus.

In certain embodiments, the method comprises agglutinating red blood cells in a volume of blood to prepare the sample. For example, the method can comprise contacting the volume of blood with an antibody to a protein produced by or otherwise related to red blood cells, e.g., an antibody to glycophorin A or with an agglutinating protein, e.g., Phytohemagglutinin E. The step of agglutinating can be performed within a microfluidic device, e.g., by introducing the volume of blood to the microfluidic device and contacting the blood with an antibody produced by or otherwise related to red blood cells or an agglutinating protein within a channel of the microfluidic device. In certain embodiments, the method comprises separating the sample of plasma and/or serum from red blood cells. In certain embodiments, the step of separating the sample of plasma and/or serum is performed without passing the plasma and/or serum through a filter. The step of separating the sample of plasma and/or serum can be performed within a portion of a microfluidic channel having generally smooth internal surfaces. For example, the portion of the microfluidic channel can have internal surfaces that are free of projections having a height in excess of about 10%, 7.5%, 5%, or about 2.5% relative to a width or height of the microfluidic channel or that are free of projections configured to retard a motion along a longitudinal axis of the microfluidic channel of red blood cells as relative to a motion along the longitudinal axis of plasma and/or serum. In certain embodiments, the step of separating the sample of plasma and/or serum is performed within a portion of microfluidic channel having at least one internal turn of at least about 90 degrees.

The microfluidic strip includes a microfluidic channel network including a sample application port and an analysis channel in fluidic communication therewith. The analysis channel includes a first reagent and a second reagent. The first and second reagents include binding agents, such as antibodies, that bind to a SARS CoV-2 antigen. As used herein, unless otherwise indicated, the term “antibody” is understood to mean an intact antibody (e.g., an intact monoclonal antibody), or a fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies).

In certain embodiments, the microfluidic device may comprise reagents for different assays in different microchannels in the same device (e.g., a microfluidic strip). For example, in certain embodiments, reagents for detecting an anti-coronavirus antibody can be present in one microchannel, and reagents for detecting a coronavirus antigen can be present in another microchannel of the same device. In certain embodiments, reagents for detecting an anti-coronavirus antibody or a coronavirus antigen can be present in one microchannel and control reagents can be present in another microchannel of the same device.

In some embodiments, one of the first reagent and the second reagent is bound to, or is configured to bind to a capture agent (e.g., a surface, such as a surface of a channel of the microchannel network, or a particle, such as a magnetic particle) and the other of the first reagent and the second reagent is bound to or is configured to bind to a detectable label. For example, the first reagent may be a conjugate including (i) a first antibody to a SARS-CoV-2 antigen (e.g., nucleocapsid) and (ii) a binding agent configured to bind to a surface or particle such as a magnetic particle. For example, the conjugate may include one of biotin, and avidin or streptavidin and the particle or surface may include the other of biotin and avidin or streptavidin, e.g., the first reagent may be a conjugate of (i) a first SARS-CoV-2 anti-nucleocapsid antibody, or fragment thereof, and (ii) biotin, and the microfluidic strip may further include a particle, e.g., a magnetic particle, conjugated to streptavidin. In another example, the conjugate may include (i) a first SARS-CoV-2 anti-nucleocapsid antibody and (ii) biotin, which is bound to a conjugate including a magnetic particle and streptavidin prior to introduction of a sample. The second reagent may be a conjugate including (i) a second antibody to the SARS-CoV-2 antigen and (ii) a detectable label such as a fluorescent particle, e.g., a fluorescent latex particle. In certain embodiments, the first SARS-CoV-2 antibody binds to a different epitope on the SARS-CoV-2 antigen than does the second SARS-CoV-2 antibody. In certain embodiments, the first reagent and/or the second reagent binds or is configured to bind a single capture agent or detectable label. In any of the above embodiments, the antibody may be a Fab.

In embodiments, a method of performing an assay to detect an antigen, e.g., a SARS-CoV-2 antigen includes combining a liquid sample, e.g., a nasal, nasopharyngeal, or saliva-based sample, which may be present in Universal Transport Media (UTM) or Viral Transport Media (VTM), suspected of containing such an antigen with a first reagent including a first antibody to a SARS-CoV-2 antigen (e.g., nucleocapsid) and a second reagent including a second antibody to a SARS-CoV-2 antigen (e.g., nucleocapsid) and determining the presence and/or amount of a complex including the first reagent, the antigen, and the second reagent. The method may include applying the liquid sample to a sample application zone of a microfluidic device. In embodiments, the sample is in a volume of between about 10 microliters and 50 microliters. In embodiments, the sample is not purified and/or concentrated prior to applying the sample to the sample application zone. The microfluidic device may include one or both of the first reagent and the second reagent in a microfluidic channel network of the microfluidic device. The first reagent may be a conjugate including (i) a first antibody to a SARS-CoV-2 antigen (e.g., nucleocapsid) and (ii) a binding agent configured to bind to a capture agent (e.g., a surface, such as a surface of a channel of the microchannel network, or a particle, such as a magnetic particle). For example, the first reagent may be a conjugate including (i) a first SARS-CoV-2 nucleocapsid antibody and (ii) biotin, and the method may further include combining the liquid sample with a third reagent including a conjugate of a magnetic particle and streptavidin. In another embodiment, the first and third reagents may be bound prior to the introduction of the sample (e.g., bound prior to being dried in the microchannel). The second reagent may be a conjugate including (i) a second SARS-CoV-2 nucleocapsid antibody and (ii) a detectable label such as a fluorescent particle, e.g., a fluorescent latex particle. In some embodiments, the first, second, and/or third reagents are disposed within an analysis channel of the microfluidic channel network. A distal portion of the analysis channel may include a gas bladder and the method may include compressing, decompressing and/or oscillating the gas bladder as disclosed herein to manipulate the liquid sample e.g., to move the liquid sample and/or mix the liquid sample and reagents as disclosed herein. The method may include magnetically retaining complexes of the third reagent, the first reagent, an antibody to SARS-CoV-2, and the second reagent in a detection zone of the microfluidic channel network prior to detecting the complexes. The method may include expelling sample liquid from the detection zone as disclosed herein prior to the detecting step.

In embodiments, the sensitivity of the SARS-CoV-2 antigen assay is at least about 96%, at least about 97%, at least about 98% or at least about 99% PPA (positive percent agreement) with a reference PCR test. In certain embodiments the SARS-CoV-2 antigen assay can detect SARS-CoV-2 antigen in a sample when the virus is present in the sample in an amount sufficient for detection of viral nucleic acid at about 28-34 RT-PCT cycles, at about 29-34 RT-PCR cycles, at about 30-34 RT-PCR cycles, at about 31-34 RT-PCR cycles, at about 32-34 RT-PCR cycles, at about 33-34 RT-PCR cycles, at about 29-33 RT-PCR cycles, at about 30-33 RT-PCR cycles, at about 31-33 RT-PCR cycles, at about 32-33 RT-PCR cycles, 29-32 RT-PCR cycles, at about 30-32 RT-PCR cycles, at about 31-32 RT-PCR cycles, about 29 about 30, about 31, about 32, about 33, or about 34 RT-PCR cycles (i.e., “Ct”). Exemplary PCR (e.g., RT-PCR) assays include, for example, the Cobas® SARS-CoV test (Roche Diagnostics, see www.fda.gov/media/136049/download) and the Abbott Real Time SARS-CoV Assay (Abbott Molecular, see www.molecular.abbott/sal/9N77-095_SARS-CoV-2_US_EUA_Amp_PI.pdf).

In embodiments, the sensitivity of the assay is at least about 96%, at least about 97%, at least about 98% or at least about 99% PPA when the sample is taken on the day of symptom onset, up to 1 day after symptom onset, up to 2 days after symptom onset, up to 3 days after symptom onset, up to 4 days after symptom onset, up to 5 days after symptom onset, up to 6 days after symptom onset, up to 7 days after symptom onset, up to 8 days after symptom onset, up to 9 days after symptom onset, up to 10 days after symptom onset, up to 11 days after symptom onset, or up to 12 days after symptom onset. In certain embodiments, the sensitivity of the assay is at least about 96%, at least about 97%, at least about 98% or at least about 99% PPA when the sample is taken between about 5 days and about 12 days after symptom onset. In certain embodiments, the limit of detection of the SARS-CoV-2 antigen assay is from about 25-35 TCID50/ml, about 28-33 TCID50/ml, about 30-33 TCID50/ml, about 31-33 TCID50/ml, about 31-32 TCID50/ml, about 32-33 TCID50/ml, or about 32 TCID50/ml.

In embodiments, a method of preparing a plasma sample includes (i) combining a blood sample, including red blood cells thereof, and an agglutinating reagent and (ii) separating, within a microchannel of a microfluidic device, the combined blood sample and agglutinating reagent into a red blood cell portion disposed in a first portion of the microchannel and a plasma portion disposed in a second portion of the microchannel. The red blood cell portion includes substantially all, e.g., essentially all of the red blood cells of the blood sample combined with the agglutinating reagent. The plasma portion is composed substantially of plasma of the blood sample combined with the agglutinating reagent, e.g., the plasma portion may consist essentially of plasma of the blood sample. The combining may be performed within the microchannel of the microfluidic device.

The blood sample may be a whole blood sample of a mammal, e.g., a human. The blood sample may be obtained from, e.g., a venous draw or finger stick. The plasma portion consisting essentially of plasma of the blood sample is a plasma sample suitable for performing a determination, e.g., an immunological determination, of the presence and/or amount of one or more targets therein. Exemplary targets include C-reactive protein (CRP), D-dimer, a member of the troponin complex such as troponin-T, troponin-I or troponin-C, glucose, and lipids such as cholesterol, HDL, or LDL. The number and amount of red blood cells that may remain in the plasma portion, if any, are few enough and of low enough concentration so as not to materially interfere with the use of the plasma portion as a plasma sample for such determinations.

The microfluidic device used in the method of preparing a plasma sample may include a liquid sample introduction port in fluidic communication with the microchannel and the microchannel may include the agglutinating reagent disposed therein. The combining may include introducing the blood sample to the microchannel via the liquid sample introduction port and flowing the whole blood along the microchannel and combining the blood sample with the agglutinating reagent disposed therein.

The separating in the method of preparing a plasma sample may include disposing the red blood cell portion and the plasma portion sequentially along a flow axis of the microchannel. The separating in the method for preparing a plasma sample may include forming a liquid-liquid interface between the red blood cell portion and the plasma portion, wherein one of the liquids of the liquid-interface is the liquid of the red blood cell portion and the other of the liquids of the liquid-liquid interface is plasma of the plasma portion. The liquids of the liquid-liquid interface may be of similar, e.g., essentially the same, composition and/or miscibility. For example, the liquid of the red blood cell portion may include residual plasma surrounding the red blood cells therein. Accordingly, in any of the embodiments the liquid-liquid interface may be substantially defined by a substantial change in the local concentration of red blood cells entrained in the liquids (with the red blood cell concentration being significantly higher in the red blood cell portion than in the plasma portion) rather than a substantial difference in the miscibility of the liquids of the two portions.

In some embodiments of the method for preparing a plasma sample, the method includes forming a distal liquid-gas interface that is disposed within the microchannel and is spaced apart from an ambient gas surrounding the microfluidic device by at least the red blood cell portion and the plasma portion, wherein the liquid of the distal liquid-gas interface is one of the red blood cell portion or the plasma portion. For example, in embodiments in which the microfluidic device includes the sample introduction port, a proximal portion, e.g., a proximal gas-liquid interface, of the blood sample may remain exposed to the ambient gas via the sample introduction port whereas the distal liquid-gas interface is spaced apart from the sample introduction port and the ambient gas by at least the red blood cell portion and the plasma portion residing within the microchannel. As another example, the microchannel may include a vent in gaseous communication with the ambient gas and the distal liquid-gas interface may be spaced apart from the vent and the ambient gas therein by at least the red blood cell portion and the plasma portion residing within the microchannel. The liquid of the distal liquid-gas interface may be plasma of the plasma portion.

In some embodiments, the method for preparing a plasma sample includes combining plasma of the plasma portion with one or more reagents disposed in the microchannel, the one or more reagents configured to interact with a target present in the plasma portion. The one or more reagents may include at least one reagent configured to participate in a reaction with the target to facilitate a determination thereof. For example, the one or more or more reagents may participate in a binding reaction with the target, e.g., an immunological reaction with the target, such as an antibody or fragment thereof configured to bind with the target. The method may further include determining presence and/or amount of the target in the plasma portion based at least in part on the interaction of the at least one reagent with the target. For example, the one or more reagents may include a detectable label, e.g., a fluorescent particle such that the binding of the reagent and target may be determined.

The method of preparing a plasma sample may include maintaining the liquid-liquid interface during the combining of the plasma of the plasma portion with the one or more reagents disposed in the microchannel. The method may further include maintaining the liquid-liquid interface during the determining the presence and/or amount of the target in the plasma portion. In some embodiments, the area of the liquid-liquid interface is essentially the same as, e.g., is defined by and the same as, the cross-sectional area of the microchannel at the location of the liquid-liquid interface within the microchannel. For example, the area of the liquid-liquid interface may be at least about 0.03 mm², at least about 0.04 mm², at least 30 about 0.06 mm², at least about 0.07 mm², or at least about 0.08 mm². The area of the liquid-liquid interface may be about 0.25 mm² or less, about 0.2 mm² or less, about 0.175 mm² or less, about 0.15 mm² or less, about 0.135 mm² or less, about 0.12 mm² or less, or about 0.1 mm² or less.

The method of separating the combined blood sample and agglutinating reagent in the method of preparing a plasma sample may include oscillating the combined blood sample and agglutinating reagent, e.g., by flowing the combined blood sample and agglutinating reagent in a first direction along the microchannel and then flowing the mixture in a second direction opposite to the first direction along the microchannel. The separating the combined blood sample and agglutinating reagent may include repeating the flowing the mixture in the first direction and then flowing the combined blood sample and agglutinating reagent in the second direction at least a number N times, e.g., wherein N is at least about 3, at least about 5, at least about 7, or at least about 10. N may be, e.g., about 20 or less, about 15 or less, or about 10 or less. Flowing the mixture in the first direction and/or in the second direction may include moving a distal liquid-gas interface of the combined blood sample and agglutinating reagent through a volume in the microchannel of at least about 0.1 μL, at least about 0.25 μL, at least about 0.35 μL, at least about 0.45 μL, or at least about 0.55 μL. Flowing the mixture in the first direction may include moving a distal liquid-gas interface of the combined blood sample and agglutinating reagent through a volume in the microchannel of about 2 μL or less, about 1.5 μL or less, about 1.2 μL or less, about 1 μL or less, about 0.9 μL or less, about 0.8 μL or less, or about 0.7 μL or less. Flowing the mixture in the first direction and/or in the second direction may include moving a distal liquid-gas interface of the combined blood sample and agglutinating reagent through a length along the microchannel of at least about 1 mm, at least about 2 mm, at least about 3 mm, at least about 4 mm, or at least about 5 mm. Flowing the mixture in the first direction and/or in the second direction may include moving a distal liquid-gas interface of the combined blood sample and agglutinating reagent through a length along the microchannel of about 10 mm or less, about 7.5 mm or less, about 6.5 mm or less, or about 5.5 mm or less. The flowing in the first direction may be performed by increasing or decreasing a pressure of the gas of the distal liquid-gas interface of the combined blood sample and agglutinating reagent and the flowing in the second direction may be performed by the other of the increasing or decreasing the pressure of the gas of the distal liquid-gas interface.

Any of the methods of preparing a plasma sample may be performed without passing the plasma portion through a filter, e.g., a membrane. The method of preparing a plasma sample may be performed without subjecting the blood sample to deterministic lateral displacement sufficient to separate the red blood cell portion and plasma portion, e.g., without subjecting the blood sample to deterministic lateral displacement. A deterministic lateral displacement approach separates particles, such as red blood cells, by flowing a particle-containing sample through an array of micro-structures or micropillars and particles differentially displaced when the flow forces particles around the obstructing micro-structures.

In any of the methods of preparing a plasma sample, internal surfaces of the portion of the microchannel within which the separating is performed may be substantially free of projections or microstructures sufficient to preferentially retain red blood cells by an amount sufficient to separate the red blood cell portion and plasma portion. The internal surfaces of the portion of the microchannel within which the separating is performed may be impermeable and lack pores through which liquid may wick by capillary action or otherwise flow through.

In any of the methods of preparing a plasma sample, the separating may be performed without subjecting the blood sample to inertial focusing sufficient to separate the red blood cell portion and plasma portion, e.g., without subjecting the blood sample to essentially any inertial focusing.

In any of the methods of preparing a plasma sample, the separating may be performed without subjecting the blood sample to centrifugal forces sufficient to separate the red blood cell portion and plasma portion, e.g., without subjecting the blood sample to essentially any centrifugal forces. The separating may be performed without rotating the microfluidic device. The separating may be performed without flowing the blood sample along a curvilinear flow path within the microchannel.

In any of the methods of preparing a plasma sample, the separating may be performed with a flow axis of the microchannel oriented substantially perpendicular to a local gravitational field of the Earth, e.g., within about 20 degrees, within about 15 degrees, within about 10 degrees, about 5 degrees of perpendicularity, or essentially perpendicular to the local gravitational field of the Earth.

In any of the methods of preparing a plasma sample, the volume of the plasma portion separated from the blood sample may be at least about 0.075 μL, at least about 0.1 μL, at least about 0.15 μL, at least about 0.175 μL, or at least about 0.2 μL. The volume of the plasma portion separated from the blood sample may be about 0.75 μL or less, about 0.65 μL or less, about 0.55 μL or less, about 0.45 μL or less, about 0.4 μL or less, about 0.35 μL or less, or about 0.325 μL or less.

In any of the methods of preparing a plasma sample, the method may be performed without combining the blood sample with an anticoagulant, e.g., heparin or EDTA. The plasma portion may be essentially free of, e.g., free of, anti-coagulants such as heparin or EDTA.

In any of the embodiments including a reagent, the reagent may be selected from the group consisting of a lysing reagent, a buffering reagent, a detectably labeled reagent (e.g., a fluorescently labeled reagent), a reagent configured to specifically bind a target to be detected, a magnetically labeled reagent, or combination thereof.

In any of the embodiments, liquid motion and/or mixing and/or oscillation of the pressure of a gas of a liquid-gas interface of a liquid may be effected by using an actuator, such as a piezoelectric actuator such as a piezoelectric bender, to compress, decompress, and/or oscillate a wall of the microfluidic device or capillary channel.

In any of the embodiments including or using a microfluidic device (e.g., strip), the microfluidic device may include multiple capillary channels, e.g., analysis channels. Each capillary channel, e.g., analysis channel, may have its own wall, e.g., a wall of a gas bladder, each independently actuable of the walls, e.g., walls of gas bladders, of other capillary channels of the microfluidic device to permit independent control over the manipulation (e.g., mixing by oscillation and/or flow) of sample liquid within the corresponding analysis channel. A reader may be configured with multiple actuators each configured to independently control the volume and/or oscillation of a corresponding gas bladder. Each of the actuators may be configured to determine a respective resonance frequency ωr of the corresponding capillary channel and gas bladder and to oscillate a wall of the capillary channel at frequency ωr as described in the above embodiments. One or more actuators of different gas bladders may be oscillated out-of-phase, e.g., in antiphase, with respect to one or more other actuators.

Whether referred to as channels, microchannels, or capillary channels, such conduits are preferably sized and configured to permit sample liquids to flow by capillary action therealong. For example, the maximum dimension of such conduits, in those portions intended to receive a liquid such as a sample liquid, may be about 2 mm or less, about 1.5 mm or less, about 1 mm or less, about 0.9 mm or less, about 0.75 mm or less, about 0.5 mm or less, about 0.25 mm or less, about 0.125 mm or less, or combination thereof along at least one, at least two, or any axis oriented perpendicular to a longitudinal axis of the conduit.

The terms “layer” and “substrate” are used synonymously herein. A layer, e.g., a substrate, of a microfluidic device may itself be composed of more than one layer, e.g., along an axis generally perpendicular to a plane of the microfluidic device. For example, substrates of a microfluidic strip may be secured (e.g., adhered) in opposition by a central layer composed of more than one layer, e.g., a central layer may include a central layer and first and second adhesive layers to which respective outer substrates are secured. Walls of a microfluidic channel network may be defined by absent, e.g., removed, portions of the central layer. A layer, e.g., a substrate, of a microfluidic device may itself be composed of more than one layer, e.g., along an axis generally parallel to a plane of the microfluidic device. For example, substrates of a microfluidic strip may be secured (e.g., adhered) in opposition by a central layer composed of multiple, e.g., first and second, separate layers spaced apart from one another with edges defining, at least in part, walls of a microfluidic channel therebetween. A microfluidic device may include one or more layers, e.g., substrates, each itself being formed of one or more layers secured together, separate layers spaced apart from one another, or combination thereof.

One or more of the layers of any of the embodiments of a microfluidic device, e.g., a microfluidic strip, may be formed of a polymer such as polyester, polydimethylsiloxane (PDMS) elastomers, thermoplastics and combinations thereof. The microfluidic strip may be formed of non-polymeric materials or of layers of different materials, e.g., with one or more rigid layers formed of, e.g., polymer, quartz or silicon, and one or more flexible layers formed, e.g., of a polymer. An adhesive layer of any of the embodiments of a microfluidic device, e.g., a microfluidic strip, may include one or more adhesive layers and such layers may comprise, e.g., an acrylic adhesive.

### BRIEF DESCRIPTION OF THE FIGURES

FIG. 1 is a perspective view of a diagnostic system of the invention including a diagnostic reader and a microfluidic strip;

FIG. 2A is a planar top view of the microfluidic strip of FIG. 1;

FIG. 2B is a side cross-sectional view of the microfluidic strip of FIG. 1, with the cross section taken along a line passing through a sample application port, along a common supply channel, branch channel and an axis a1 of an analysis channel of the microfluidic strip as shown in FIG. 2A;

FIG. 3 is a planar cross-sectional view showing a second reagent zone of the microfluidic strip of FIG. 1, with a sample liquid present therein;

FIG. 4 illustrates a partial view of a piezoelectric actuator of the reader of FIG. 1 and a partial view of the microfluidic strip of FIG. 1 operationally disposed with respect thereto;

FIG. 5 is a side cross-sectional view of the piezoelectric actuator and microfluidic strip taken along the line A-A of FIG. 4 (aligned with axis a1);

FIG. 6 is a planar top view of a second embodiment of a microfluidic strip of the invention;

FIG. 7 is a perspective exploded view of the microfluidic strip of FIG. 6;

FIG. 8 is a top partial cross-sectional view of a first reagent zone of the microfluidic strip of FIG. 6 taken along the line 8 in FIG. 9;

FIG. 9 is a side partial crossectional view of the first reagent zone of FIG. 8 taken along the line 9 therein;

FIG. 10 is a perspective cut-away view of a fill electrode and analysis channel of a microfluidic strip of the invention;

FIG. 11 is a planar view of the fill electrode and microchannel of FIG. 10 taken along the line 11 therein; and

FIG. 12 is a planar top view of an embodiment of a microfluidic strip of the invention;

FIG. 13A is a planar top view of an embodiment of a microfluidic strip of the invention; FIG. 13B is a perspective exploded view of the microfluidic strip of FIG. 13A; FIG. 13C is a partial planar view of the upper substrate and adhesive layer of the strip of FIG. 13A (the lower substrate not shown) viewed from beneath, through the adhesive layer to the upper substrate within the section 13c shown in FIG. 13A; and FIG. 13D is a partial planar top view of the strip of FIG. 13A within the section 13d shown FIG. 13A.

FIG. 14 depicts a top planar view of an embodiment of a microfluidic device of the invention for preparing a plasma sample from a blood sample and determining the presence or amount of CRP in the plasma sample.

FIG. 15 is a photograph of a portion of a microfluidic device as shown in FIG. 14, showing a separation of whole blood into a plasma portion and a red blood cell portion.

FIG. 16 depicts an embodiment of a SARS-CoV-2 Ab strip having, proceeding upward from lower left, a sample application zone, a tapered common supply channel, a branch channel, and, proceeding from right to left along the branch channel, four analysis channels and a hematocrit channel, the proximal portion of which includes an excitation electrode and a common electrode

FIG. 17A depicts an embodiment of the S1-S1 Bridge Serology assay components (left of arrow) and immune-complex formation (right of arrow). FIG. 17B depicts an embodiment of the RBD-S1 Bridge Immunoassay.

FIG. 18 depicts an embodiment of the On-Board Control assay.

FIG. 19 depicts an embodiment of a strip having, proceeding upward from lower left, a sample application zone, an arcuate common supply channel, a branch channel, and, proceeding from right to left in the Figure, four analysis channels, a common electrode, an excitation electrode, and a narrow vent channel terminating in a vent.

FIG. 20 depicts an embodiment of the SARS-CoV-2 Ag Nucleocapsid Protein Immunoassay—Channels 2 and 3.

FIG. 21 depicts an embodiment of the RBD-IgA Serology Assay—(Optionally Reported)—Channel 1.

FIG. 22 depicts an embodiment of the On Board Control Assay—Channel 4.

FIG. 23 depicts a schematic for “RBD-IgA Serology Assay—(Optionally Reported)—Channel 1”.

FIG. 24 depicts an embodiment of the On Board Control Assay, wherein the strip comprises a fluorescent latex particle-biotin conjugate pre-bound to a conjugate of streptavidin and a magnetic particle.

FIG. 25A depicts the limit of detection (LoD) of each test for Serial 2-fold dilutions of the characterized SARS-CoV-2 aliquots, with the LoD being the lowest concentration at which all replicates were positive being treated as the LoD for each test.

FIG. 25B depicts a dilution series to determine the LoD of SARS-CoV-2 Culture Fluid Heat Inactivated Virus, indicating that the LoD is in the range 1:6400-1:12800 dilution i.e. 118-236 TCID50/ml.

FIG. 26 depicts analysis of high dose hook effect was observed up to 1.4×10, TCID50/mL of gamma-irradiated SARS-CoV-2 with the SARS-CoV-2 Ag Test.

FIG. 27 depicts cumulative True Positives (TP) and False Negatives (FN) for the test over a 12 day period since SARS-CoV-2 (COVID-19) symptom onset.

FIG. 28 shows a plot of RT-PCR cycle time (“Ct”) for samples collected a given number of days after SARS-CoV-2 (COVID-19) symptom onset.

### DETAILED DESCRIPTION

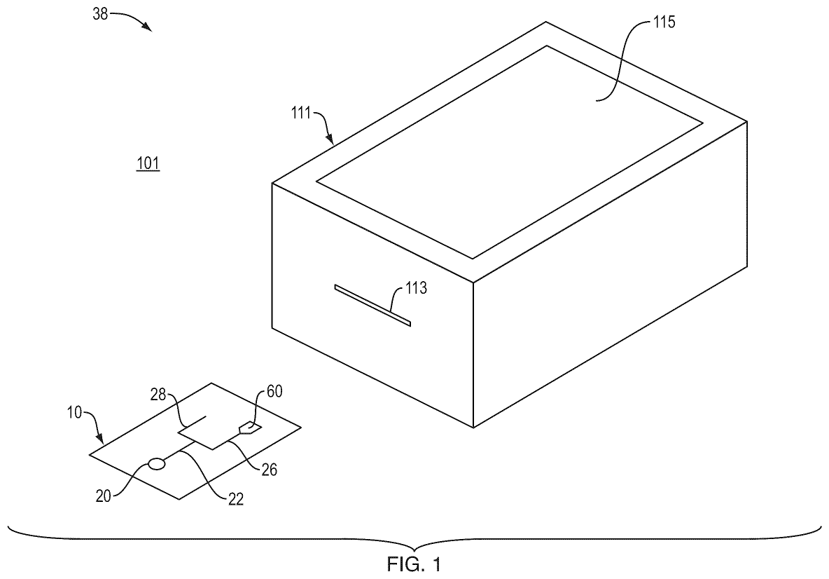

With reference to FIG. 1, a diagnostic system 101 includes a diagnostic reader 111 and a microfluidic strip 10. Reader 111 operates strip 10 to determine the presence and/or amount of at least one target (e.g., a biomolecule such as a protein) present in a sample liquid applied to strip 10. Reader 111 also operates strip 10 to determine a physiochemical property, e.g., a hematocrit, of a sample liquid applied to strip 10. Reader 111 includes an input port 113, which receives microfluidic strip 10, and a touchscreen 115 by which a user can enter and receive information relevant to the operation of reader 111 and the determination of the target. Elements of strip 10 are discussed first before turning to elements of reader 111.

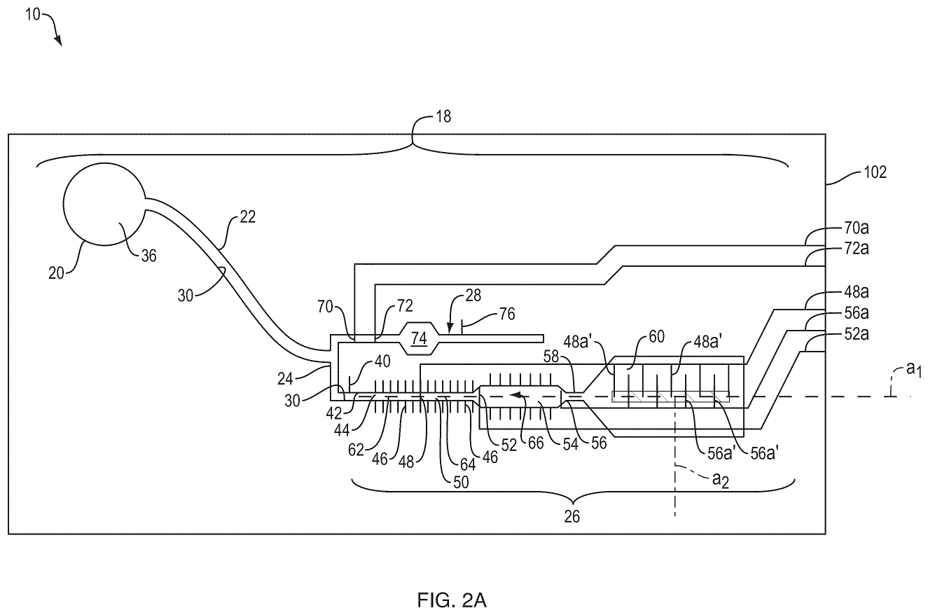

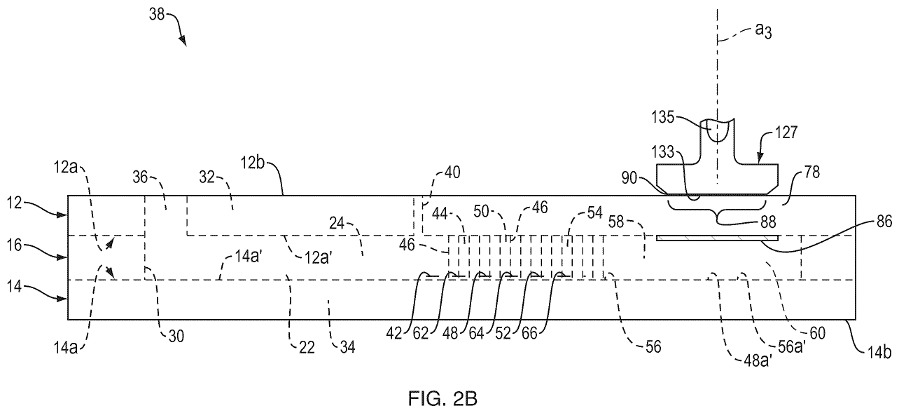

With reference to FIGS. 2A and 2B, strip 10 includes an upper substrate 12 and a lower substrate 14 each composed of 100 μm thick polyester film. A lower surface 12a of upper substrate 12 and an upper surface 14a of lower substrate 14 are adhered in opposition by an adhesive layer 16, 110 μm thick. Adhesive layer 16 occupies less than all of the area of surfaces 12a,14a between upper and lower substrates 12,14 to define a microfluidic channel network 18. Microfluidic channel network 18 has a sample application zone 20, a common supply channel 22, a branch channel 24, an analysis channel 26, and a hematocrit channel 28. Microfluidic channel network 18 has side walls 30 defined by adhesive layer 16, an upper wall 32 defined by those portions of upper substrate 12 unoccupied by adhesive layer 16, e.g., overlying absent portions of adhesive layer 16, and a lower wall 34 defined by those portions of lower substrate 14 unoccupied by adhesive layer 16, e.g., underlying the absent portions of adhesive layer 16. Upper wall 32 has an inner surface 12a′ defined by those portions of surface 12a unoccupied by, e.g., exposed by the absent portions of, adhesive layer 16. Lower wall 34 has an inner surface 14a′ defined by those portions of surface 14a unoccupied by, e.g., exposed by the absent portions of, adhesive layer 16. Upper substrate 12 has an outer (upper) surface 12b and lower substrate 14 has an outer (lower) surface 14b.

Sample application zone 20 is a port 36 extending through upper substrate 12 and adhesive layer 16 of microfluidic strip 10 and defines a proximal origin of microfluidic channel network 18. Port 36 places the channels of microfluidic channel network 18 in gaseous communication with a gas, e.g., air, of surrounding ambient atmosphere 38. Sample liquid (e.g., blood) applied to sample application zone 20 via port 36 flows by capillary action along common supply channel 22 to branch channel 24 along which a first portion of the sample liquid flows by capillary action to analysis channel 26 and a second portion of the sample liquid flows by capillary action to hematocrit channel 28.

Hematocrit channel 28 is arranged and configured to facilitate a reagent-free optical determination of the hematocrit of a liquid sample of blood applied to sample application zone 20. Proceeding distally from branch channel 24, hematocrit channel 28 includes a supply electrode 70, a hematocrit fill electrode 72, a hematocrit detection zone 74, and a vent 76. Portions of hematocrit channel 28 disposed proximally and distally to hematocrit detection zone 74 each have a height of 110 μm, and a width of 670 μm. Hematocrit detection zone 74 has a height of 110 μm, a width of 2300 μm, and a length of 3 mm. Operation of the hematocrit determination is further described below.

Analysis channel 26 is arranged and configured to facilitate the determination of the presence and/or amount of the target present in the sample liquid. Proceeding distally from branch channel 24 along a longitudinal axis a1 of analysis channel 26, analysis channel 26 includes a vent 40, a capillary stop 42, a first reagent zone 44, a plurality of side cavities 46, a first fill electrode 48, a second reagent zone 50, a second fill electrode 52, a detection zone 54, a third fill electrode 56, a spacing channel 58, and a gas bladder 60.

Common supply channel 22, branch channel 24, first reagent zone 44, second reagent zone 50, and spacing channel 58 each have a height of 110 μm and a width of 670 μm. First reagent zone 44 and second reagent zone 50 each have a length of 4.4 mm and a volume of about 324 nL. Detection zone 54 has a height of 110 of μm, a width of 1500 μm, a length of 5.4 mm and a volume of about 890 nL. Spacing channel 58 has a length of 1 mm. The total volume of analysis channel 26 between capillary stop 42 and third fill electrode 56 is about 1.6 μL. Gas bladder 60 has a height of 110 of μm, a width of 5.5 mm, a length of 11.4 mm and a volume of about 6.9 μL. The aforementioned dimensions, e.g., widths, of portions of analysis channel 26 exclude side cavities 46, which are further discussed below.

First reagent zone 44 includes a lysing reagent 62 deposited therein upon lower surface 14a′. Lysing reagent 62 is configured to lyse cells present in the sample liquid releasing target present within the intracellular material. Second reagent zone 50 includes a labeled binding reagent 64 deposited therein upon lower surface 14a′. Labeled binding reagent 64 has a first portion (e.g., an antibody) that specifically binds the target and a second portion that is a detectable fluorescent label. The binding of the target and labeled binding reagent 64 forms a first complex. Detection zone 54 includes a magnetic binding reagent 66 deposited therein upon lower surface 14a′. Magnetic binding reagent 66 has a first portion (e.g., an antibody) that binds the first complex and a second portion that is a magnetic particle. The binding of the first complex and the magnetic binding reagent 66 forms a second complex.

Each of the reagents 62,64,66 is in dry (e.g., lyophilized) form. Once the manufacturing of strip 10 is complete (e.g., after the deposited reagents 62,64,66 have dried within microfluidic channel network 18 and upper and lower substrates 12,14 have been secured, e.g., adhered, together by adhesive layer 16), strip 10 is free of liquids (e.g., strip 10 does not include a stored liquid reagent such as a buffer). In use, the only liquid applied to strip 10 is a sample liquid containing a target to be determined. Strip 10 is configured to not require, e.g., not configured to permit, the introduction of a liquid other than the sample liquid containing the target to be determined.

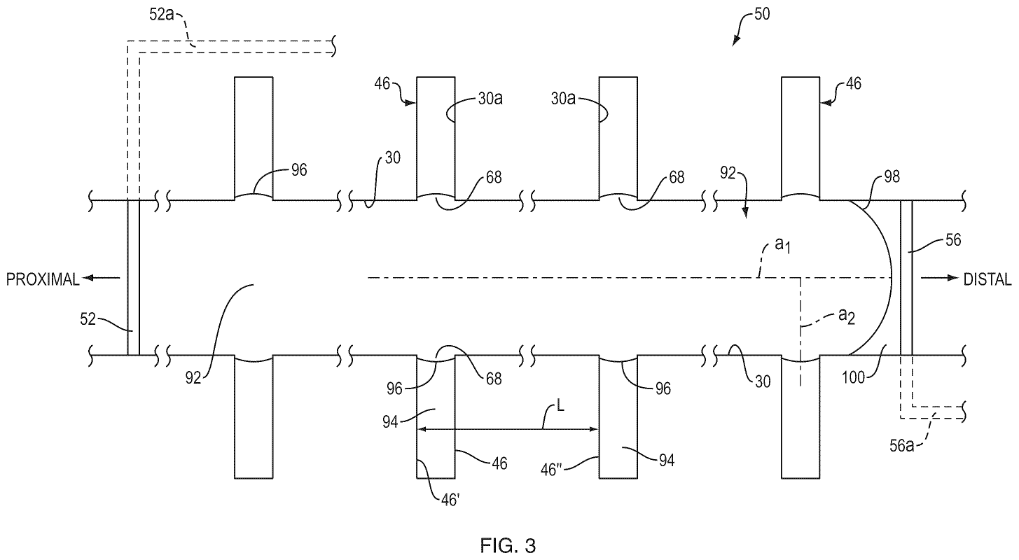

As discussed above, and with further reference to FIG. 3, analysis channel 26 includes a plurality of side cavities 46 located within side walls 30 of first reagent zone 44, second reagent zone 50 and detection zone 54. Side cavities 46 have side walls 30a defined by the portions of adhesive layer 16 which are absent, e.g., removed, from between upper and lower substrates 12,14 and upper and lower walls (not shown) respectively defined by respective portions of surfaces 12a,14a of upper and lower substrates 12,14 overlying and underlying the absent portions of adhesive layer 16. Each side cavity 46 has a height of 110 μm, a width of 75 μm along longitudinal axis a1 of analysis channel 26, a depth of 700 μm along an axis a2 oriented perpendicular to longitudinal axis a1 and a volume of 5.8 nL. Side cavities 46 are spaced apart from one another by a distance of 700 μm along longitudinal axis a1 of analysis channel 26. Each side cavity 46 has a single opening 68 that faces analysis channel 26 and opposes an opening 68 of a side cavity 46 disposed within the opposite side wall 30 of analysis channel 26.

With reference to FIG. 3, a length L oriented generally along axis a1 extends from a proximal wall 46′ of a first side cavity 46 to a proximal wall 46” of the adjacent distal cavity 46. Within each of first and second reagent zones 44,50, along a portion of the capillary channel having the length L, a ratio of a total volume of side cavities 46 (2×5.8 nL) to a total volume of analysis channel 26 excluding the volume of side cavities 46 (57 nL) is 0.20. Within detection zone 54, along a length correspondingly disposed and oriented to length L along axis a1, a ratio of a total volume of side cavities 46 (2×5.8 nL) to a total volume of analysis channel 26 excluding side cavities 46 (128 nL) is 0.09. Within each of first and second reagent zones 44,50, along length L, a ratio of a total area of openings 68 of side cavities 46 (2×8250 μm²) to a total internal surface area of analysis channel 26 excluding openings 68 (2×77,000 μm²+2×519,250 μm²) is 0.0138. Within detection zone 54, along a length correspondingly disposed and oriented to length L along axis a1, a ratio of a total area of openings 68 of side cavities 46 (2×8250 μm²) to a total internal surface area of analysis channel 26 excluding openings 68 (2×77,000 μm²+2×1,162,500 μm²) is 0.0067.

Other than opening 68, side cavities 46 lack any means of ingress/egress for gas and liquids and are otherwise sealed with respect to channel network 18 and surrounding ambient atmosphere 38. A sample liquid 92 passing along analysis channel 26 is prevented from completely entering a side cavity 46 by surface tension and the gas pressure of gas 94 within side cavity 46, which gas pressure increases as sample liquid begins to enter side cavity 46.

Therefore, the sample liquid within analysis channel 26 and the gas 94 within each side cavity 46 form a gas-liquid interface 96 adjacent analysis channel 26. Each gas-liquid interface 96 has an axis of symmetry generally aligned with axis a2. The interaction of side cavities 46 and sample liquid is further discussed below. In FIG. 3, sample liquid 92 has solubilized labeled binding reagent 64 disposed in second reagent zone 50 and, therefore, labeled binding reagent 64 is not shown. FIG. 3 also illustrates a distal liquid-gas interface 98 formed by sample liquid 92 and a gas 100 present in portions analysis channel 26 disposed distally to sample liquid 92. Distal liquid-gas interface 98 is the liquid-gas interface of the sample liquid within analysis channel 26 that is spaced apart from sample application zone 20 by the sample liquid. Distal liquid-gas interface 90 has an axis of symmetry generally aligned with longitudinal axis a1. As discussed below, the position of distal liquid-gas interface 98 changes as the determination of the target proceeds.

Gas bladder 60 defines a distal terminus of analysis channel 26. The portion of upper wall 32 overlying gas bladder 60 defines a gas bladder upper wall 78 and the portion of lower wall 34 underlying gas bladder 60 defines a gas bladder lower wall 84. Gas bladder 60 is in gaseous communication with surrounding ambient atmosphere 38 only via (i) analysis channel vent 40 via analysis channel 26, (ii) hematocrit channel vent 76 via analysis channel 26, branch channel 24 and a proximal portion of hematocrit channel 28, and (iii) port 36 via analysis channel 26, branch channel 24 and common supply channel 22. Once manufacturing of strip 10 is complete, strip 10 is typically packaged within a hermetically sealed package, e.g., a foil pouch. Upon opening strip 10 in preparation for use, gas within microfluidic channel network 18 is free to exchange with gas of surrounding ambient atmosphere 36.

Other than aforementioned vents 40,76 and port 36, microfluidic channel network 18 lacks any other port or route of gas ingress or egress to or from surrounding ambient atmosphere 38 and is otherwise sealed with respect to surrounding ambient atmosphere 38. Microfluidic channel network 18 also lacks any port or other route by which a gas could be introduced to or withdrawn from microfluidic network 18 via a source of gas external to microfluidic strip 10. Therefore, in the absence of sample liquid disposed within microfluidic channel network 18 between gas bladder 60 and port 36 and vents 40,76, a pressure increase within gas bladder 60 (e.g., created by a reduction of the volume of gas bladder 60 as by compression of gas bladder upper wall 78 toward gas bladder lower wall 84) expels gas disposed therein proximally along analysis channel 26, branch channel 24, and common supply channel 22 toward and out of port 36 and, to a lesser extent, out of vents 40 and 76. In the absence of sample liquid disposed within microfluidic channel network 18 between gas bladder 60 and port 36 and vents 40,76, a pressure decrease within gas bladder 60 (e.g., created by an increase of the volume of gas bladder 60 as by retraction of gas bladder upper wall 78 away from gas bladder lower wall 84) draws gas distally from surrounding ambient atmosphere 38 through port 36 and, to a lesser extent, through vents 40,76 into microfluidic network 18 toward and into gas bladder 60. Because the cross-sectional areas of vents 40,76 are significantly smaller than the cross-sectional area of port 36, the primary route of gas ingress/egress to or from microfluidic channel network upon the compression/expansion of gas bladder 60 is via port 36.

As discussed above with reference to FIG. 3 and further discussed below, sample liquid disposed in microfluidic channel network 18 between port 36 and gas bladder 60 creates a liquid-gas interface 98 disposed at a distal terminus of sample liquid 92 and proximally to gas bladder 60. The compression and retraction of gas bladder upper wall 78 respectively increases and decreases the gas pressure acting upon the liquid-gas interface and provides the ability to control the flow and/or mixing of sample liquid in the microfluidic channel network 18.

The electrodes of strip 10 are disposed and configured to permit reader 111 to monitor the proper filling of strip 10 with sample liquid, the proper movement of sample liquid within strip 10 and the operation (e.g., the compression state) of gas bladder 60. Each of supply electrode 70 and fill electrodes 48,52,56,72 is disposed on internal surface 14a′ of lower wall 34 in a location that sample liquid within microchannel network 18 will contact the electrode. Each of analysis channel fill electrodes 48,52,56 is connected via a respective lead 48a,52a,56a to a distal periphery 102 of strip 10. Hematocrit channel supply electrode 70 and fill electrode 72a are each connected via a respective lead 70a,72a to distal periphery 102. When strip 10 is fully inserted into reader 111, distal termini of leads 48a,52a,56a,70a,72a engage corresponding contacts (not shown) within reader 111. The engaged contacts permit reader 111 to deliver and/or receive electrical signals to and/or from supply electrode 70 and fill electrodes 48,52,56,72. Except as discussed below, corresponding leads 48a,52a,56a,70a,72a are disposed outside of microfluidic channel network 18 on those portions of upper surface 14a of lower substrate 14 that remain covered by adhesive layer 16.

With reference to FIG. 2A, portions of lead 48a of first fill electrode 48 and of lead 56a of third fill electrode 56 pass along internal surface 14a′ of gas bladder lower wall 84 and respectively define interposed first and second interposed electrically conductive lead electrodes 48a′ and 56a′. An electrically conductive bridging contact 86 is disposed on internal surface 12a′ of gas bladder upper wall 78 and overlies lead electrodes 48a′,56a′. When gas bladder upper wall 78 is fully compressed, as discussed below, bridging contact 86 establishes continuity between lead electrodes 48a′,56a′, which are otherwise are not in direct continuity with one another. Reader 111 delivers and/or receives electrical signals to and/or from lead electrodes 48a′,56a′ via the same contacts as for fill electrodes 48,56.

Reader 111 and strip 10 are configured to permit reader 111 to determine when strip 10 has been fully inserted into reader 111. For example, reader 111 and strip 10 may incorporate any of the exemplary structures and techniques for determine proper insertion of a strip into a reader as disclosed in International application no. PCT/GB2017/051946 filed 30 Jun. 2017 the (“'946 application”), which application is incorporated by reference in its entirety.

Reader 111 includes a magnetic field generator (not shown) to control the movement and/or positioning of magnetic binding reagent 66. The magnetic field generator may incorporate any of the exemplary structures and techniques for magnetically controlling the movement and/or position of magnetic reagents as disclosed in International application no. PCT/GB2019/053207 filed 12 Nov. 2019, which is incorporated by reference in its entirety. The magnetic field generator includes a permanent magnet at the end of a pivot arm configured to move the permanent magnet between a first and second position. In the first position, the magnet is displaced from detection zone 54 such that detection zone 54 does not experience a magnetic field sufficient to substantially influence the magnetic particles of magnetic binding reagent 66 therein. In the second position, the magnet is disposed beneath lower substrate 14 underlying detection zone 54 such that the magnetic particles of magnetic binding reagent 66 experiences a magnetic field that forces the magnetic particles toward lower surface 35 of lower substrate 14 within detection zone 54. The force is sufficient to substantially retain magnetic binding reagent 66 within detection zone 54 in the presence of the flow and/or mixing of sample liquid as induced by a flow controller (as discussed below). With the strip inserted and a liquid sample not yet applied, reader 111 positions the magnetic field generator in the first position.

Reader 111 includes an optical detection system (not shown) having a light source configured to irradiate detection zone 54 with light at a wavelength selected to excite fluorescence from the detectable label of labeled binding reagent 64 and an optical detector configured to detect fluorescence emitted therefrom. The optical detection system can include any of the exemplary structures and techniques for optical detection as disclosed in abovementioned '946 application.

To facilitate hematocrit determination, reader 111 includes two light emitting diodes (LED's) (not shown), one of which emits in the cyan (506 nm) and the other in the infrared region (805 nm). With strip 10 fully inserted into reader 111, the LED's are disposed above hematocrit detection zone 74 and configured to transmit light through a blood sample disposed therein. Diagnostic reader also includes a photodiode (not shown) configured to detect light transmitted through hematocrit detection zone 74. Hemoglobin absorbs strongly in the cyan (506 nm) whereas the infrared light at 805 nm is less strongly absorbed by hemoglobin and therefore permits correction for scattering and turbidity within the sample. The short optical path length determined by the height of the hematocrit detection zone (110 μm) permits the absorbance of hemoglobin to be measured in undiluted whole blood.

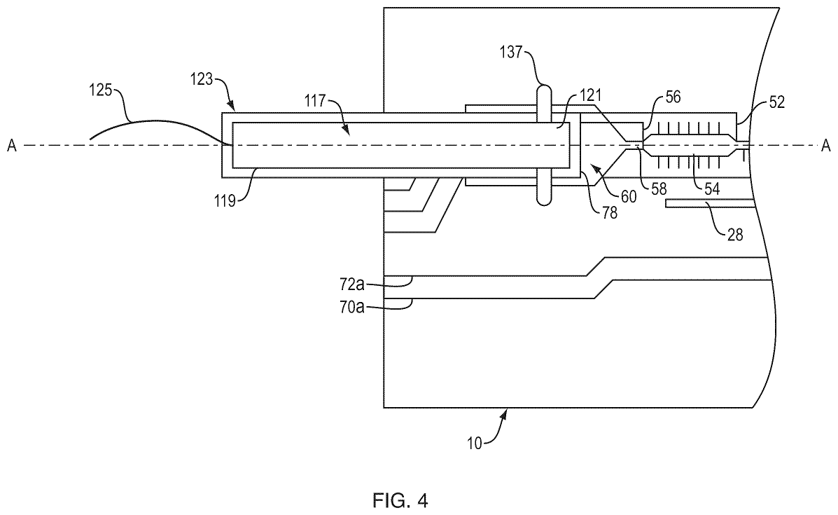

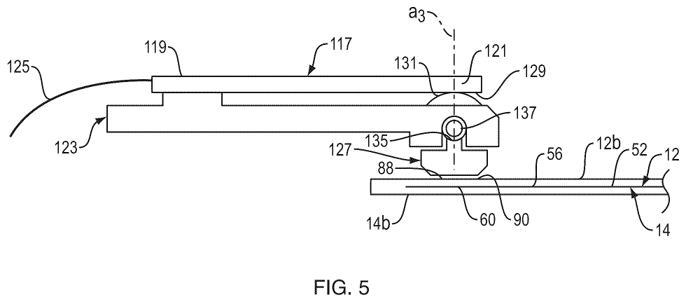

Reader 111 also includes a flow controller disposed therein. With reference to FIGS. 4 and 5, the flow controller includes an actuator such as a piezoelectric bender 117, which is an arm extending from a fixed end 119 to an actuation end 121. Piezoelectric bender 117 has a length along axis a1 of 30 mm and a width along axis a2 (defined below) of 5 mm (axes a1 and a2 are shown in FIG. 2A). Fixed end 119 is fixed to a mounting block 123 and is electrically coupled to an electrical connection 125, by which reader 111 provides electrical actuation signals to bender 117. Actuation end 121 is responsive to the electrical signals, which control the position and motion of actuation end 121 along an axis a3 oriented generally perpendicular to the plane of microfluidic strip 10 (perpendicular to axes a1 and a2). In turn, the position and motion of actuation end 117 controls the position and motion along axis a3 of an actuation foot 127.

Actuation foot 127 is mounted within mounting block 123 beneath actuation end 121 via a mounting pin 137 of mounting block 123 that passes through a slot 135 within actuation foot 127. The mounting permits actuation foot 127 to move freely with respect to mounting block 123 along axis a3. Actuation foot 127 has an upper surface 131, a lower surface 133 and a total height of 8 mm therebetween along axis a3. Upper surface 131 is disposed beneath a lower surface 129 of actuation end 121 of piezoelectric bender 117. Lower surface 133 is configured to transmit the motion of actuation end 121 to gas bladder upper wall 78 of strip 10. When strip 10 is fully inserted into reader 111, lower surface 133 of actuation foot 127 contacts a contact portion 88 of outer surface 12b of gas bladder upper wall 78. Contact portion 88 has a length (along axis a1 generally aligned with the length of analysis channel 26 and gas bladder 60) of 5 mm and a width (along axis a2 generally perpendicular to axis a1 and the length of analysis channel 26 and gas bladder 60) of 1 mm. The area of contact portion 88 is about 8% of the total area of outer surface 12b of gas bladder upper wall 78 overlying gas bladder 60. Outer surface 14b of lower substrate 14 of strip 10 rests upon a strip support (not shown) within reader 111. The strip support prevents lower substrate 14 including lower wall 34 from deflecting downward along axis a3 in response to downward motion of actuation foot 127 (i.e., motion along axis a3 toward strip 10) that compresses gas bladder upper wall 78 as discussed below.

Contact portion 88 is spaced apart laterally from and distal to third fill electrode 56 along axis a1. Therefore, contact portion 88 is spaced apart laterally from locations of analysis channel 26 occupied by sample liquid during operation of microfluidic strip 10. For 25 example, when sample liquid occupies first reagent zone 44 as determined by first fill electrode 48 but has not yet progressed further distally along analysis channel 26, a distance along axis a1 between distal liquid-gas interface 98 and a proximal-most location 90 of contact portion 88 is about 15 mm. When sample liquid occupies second reagent zone 50 as determined by third fill electrode 56 but has not yet progressed further distally along analysis channel 26, a distance along axis a1 between distal liquid-gas interface 98 and a proximal-most location 90 of contact portion 88 is about 10 mm. When sample liquid occupies detection zone 60 as determined by hematocrit fill electrode 72, the sample liquid is at its distal-most position within analysis channel 26 and a distance along axis a1 between distal liquid-gas interface 98 and a proximal-most location 90 of contact portion 88 is about 5 mm.

When reader 111 senses that strip 10 is fully inserted and prior to the application of sample liquid to port 36, reader 111 actuates the flow controller causing piezoelectric bender 117 to press lower surface 129 of actuation end 121 against upper surface 131 of actuation foot 127. The applied pressure drives actuation foot 127 downward along axis a3 causing lower surface 133 of actuation foot 127 to compress gas bladder upper wall 78 toward the underlying gas bladder lower wall 84. The compression places gas bladder upper wall 78 under tension and causes outer surface 12b of gas bladder upper wall 78 to become generally concave and internal surface 12a′ of gas bladder upper wall 78 to become generally convex. Upper substrate 12 including gas bladder upper wall 78 is sufficiently flexible to permit the compression and relaxation of upper wall 78 over a distance corresponding to the height of gas bladder 60.

The flow controller continues to compress gas bladder upper wall 78 until bridging contact 86 on internal upper surface 12a′ of gas bladder upper wall 78 contacts lead electrodes 48a′,56a′ on internal lower surface 14a′ of gas bladder lower wall 84 placing lead electrodes 48a′,56a′ in electrical continuity. Reader 111 receives a signal via leads 48a,56a that lead electrodes 48a′,56a′ are in continuity indicating that upper wall portion 78 overlying gas bladder 60 has been fully compressed. The flow controller then reverses the actuation of piezoelectric bender 117 to retract actuation end 121 vertically to reduce compression of gas bladder upper wall 78. Because gas bladder upper wall 78 has been placed under tension, the reduced compression causes gas bladder upper wall 78 to retract vertically against lower surface 133 of actuation foot 127, pushing actuation foot 127 vertically along axis a3, separating bridging contact 86 from lead electrodes 48a′,56a′, and breaking continuity between leads 48a,56a. The piezoelectric actuator continues to reduce compression of upper wall portion 78 only until a signal at leads 48a,56a indicates that continuity between lead electrodes 48a′,56a′ is broken. Once the broken continuity signal is received, the piezoelectric actuator ceases further motion of actuation end 121 and causes the actuation end 121 and actuation foot 127 to maintain compression of upper wall portion 78 overlying gas bladder 60 with bridging contact 86 and lead electrodes 48a′,56a′ just separated (e.g., by about 2.5 μm). Gas bladder 60 is then in an operationally fully compressed state, with upper wall portion 78 generally concave and under tension with contact portion 88 pressing against lower surface 133 of actuation foot 127, upper surface 131 of actuation foot 127 pressing against lower surface 129 of actuation end 121, and with only a slight separation of bridging contact 86 and lead electrodes 48a′,56a′.

The step of allowing upper wall portion 78 to retract slightly from the underlying portion of lower substrate 14 thereby providing the slight separation between bridging contact 86 (which is disposed on internal surface 12a′ of upper wall portion 78) and lead electrodes 48a′,56a′ (which are disposed on the opposed internal surface 14a′ of lower substrate 14) provides several functions. For example, as discussed below, first fill electrode 48 operates to sense the presence of sample liquid at a distal terminus of first reagent zone 44 and third fill electrode 56 operates to sense the presence of sample liquid at a distal terminus of detection zone 54. If bridging contact 86 maintained electrical continuity between lead electrodes 48a′,56a′ (and, therefore, continuity between leads 48a,56a and fill electrodes 48,56), fill electrodes 48,56 would not function to independently sense the presence of sample liquid. Breaking continuity between lead electrodes 48a′,56a′ permits fill electrodes 48,56 to perform their respective sample liquid sensing functions. A single pair of leads (48a,56a), therefore, permits the performance of two separate (independent) liquid sensing functions (e.g., determining the presence of sample liquid at two respective locations via electrodes 48,56) and a mechanical sensing function (e.g., gas bladder compression via lead electrodes 48a′,56a′). Likewise, reader 111 requires only one pair of contacts to engage leads 48a,56a and receive corresponding electrical signals indicative of the sample liquid sensing and mechanical sensing. Strip 10 and reader 111 are therefore less expensive and complex to manufacture than if a separate pair of independent electrodes and leads were used to sense the state of compression of gas bladder 60.

In addition, during the compression of upper wall portion 78, reader 111 receives calibration signals from the piezoelectric actuator indicative of the extent of compression required to fully compress upper wall portion 78 and to position gas bladder 60 in the operationally fully compressed state. Reader 111 also receives calibration signals indicative of the amount of force that is required to be applied by the piezoelectric actuator in order to displace upper wall portion 78 of gas bladder 60. Reader 111 stores the calibration signals and can therefore operate the piezoelectric actuator to return the gas bladder 60 to the operationally fully compressed state and/or achieve a given displacement of upper wall portion 78 even without further signals from lead electrodes 48a′,56a′. Such capability is advantageous as sample liquid subsequently introduced to analysis channel 26 during operation of strip 10 (as discussed below) may place electrodes 48,56 in continuity rendering lead electrodes 48a′,56a′ inoperative or unreliable in sensing the compression state of gas bladder 60.

The retraction of upper wall portion 78 also ensures that upper wall portion 78 will move without lag time (e.g., to expand or further compress), in response to movement of actuation foot 127. Because the expansion and compression of upper substrate 78 is used to control movement and/or mixing of sample liquid within analysis channel 26 (as discussed below) the movement of upper substrate 78 without lag time ensures that the controlled movement and/or mixing of sample liquid occurs without lag time in response to actuation by the piezoelectric actuator. If the step of slightly separating the upper wall portion 78 from the underlying portion of lower substrate 14 had not been performed, an uncertain amount of retraction of actuation foot 127 would have to occur prior to the occurrence of separation and the initiation of movement of upper wall portion 78 with the consequent change in volume of gas bladder 60. Accordingly, the occurrence of a gas pressure change (e.g., a steady change or an impulse) within gas bladder 60 effecting movement or mixing of sample liquid within analysis channel 26 would also be delayed. By “without lag time” it is meant that the response of upper wall portion 78 is limited substantially by the physical properties of upper wall portion 78 (e.g., the elastic modulus thereof) and the mechanics of actuation foot 127 rather than the need to reverse excess compression of upper wall portion 78 against underlying lower substrate 14 that may have resulted during the initial compression step.

After the step of positioning gas bladder 60 in the operationally fully compressed state, sample application zone 20 (port 36) remains in gaseous communication with surrounding ambient atmosphere 38 and, without any sample liquid occupying microchannel network 18, gas bladder 60 and the rest of microchannel network 18 are in gaseous communication with and at the same gas pressure as the gas of ambient atmosphere 38 surrounding reader 111 and microfluidic strip 10. The volume of gas displaced from gas bladder 60 by placing gas bladder 60 in the operationally fully compressed state as compared to the fully relaxed state is about the same as the volume of analysis channel 26 between branch channel 24 and third fill electrode 56.

Continuing with the determination of the target, and with strip 10 fully inserted into input port 113 of reader 111, the magnetic field generator in the first position, and gas bladder 60 in the operationally fully compressed state, the operator applies a sample liquid (e.g., blood) to sample application zone 20 of strip 10. The total volume of the applied sample is between 2.5 and 7.5 μL. The sample liquid flows through port 36 and by capillary action along common supply channel 22 until reaching branch channel 24 at which point the sample liquid splits with a first portion proceeding along branch channel 24 toward hematocrit channel 28 and a second portion proceeding along branch channel 24 toward analysis channel 26. The first portion of sample liquid proceeds to hematocrit channel 28 until the corresponding distal liquid-gas interface of the sample liquid (i.e., the liquid-gas interface of the sample liquid within hematocrit channel 28 that is spaced apart from sample application zone 20 by the aliquot of sample liquid within hematocrit channel 28, branch channel 24 and common supply channel 22) just passes hematocrit channel vent 76. Because the small portion of hematocrit channel 28 disposed distally to vent 76 does not provide any route for gas ingress/egress, gas pressure buildup distal to the sample liquid then causes the sample liquid ceases flowing along hematocrit channel 28. The second portion of sample liquid proceeds until distal liquid-gas interface 98 of the sample liquid (i.e., which is spaced apart from sample application zone 20 by the aliquot of sample liquid within analysis channel 26, branch channel 24 and common supply channel 22) just passes analysis channel vent 40 and contacts capillary stop 42 at which location the sample liquid ceases flowing along analysis channel 26. With the sample liquid liquid-gas interfaces at the locations set forth in the previous two sentences, strip 10 has been properly filled with sample liquid and is ready to continue determine the presence and/or amount of target present in the sample liquid.

Reader 111 is configured to determine the occurrence (or not) of the proper filling of strip 10 with sample liquid as well as the presence of sample liquid at locations within microfluidic channel network 18 corresponding to fill electrodes 48,52,56,72. When strip 10 is fully inserted into reader 111, reader 111 applies an electrical “supply” signal (e.g., a time varying signal such as a square wave or other periodic signal) to supply electrode lead 70a of supply electrode 70. The time varying signal typically has an offset, e.g., a DC offset, so that the signal does not fall to zero or below zero volts with respect to ground. In addition, a maximum potential of the time varying signal is less than a potential that would cause coagulation or adverse chemical reactions to occur within a liquid sample (e.g., a blood sample). An exemplary time varying signal is a square wave with a peak-to-peak magnitude of between 0.25 and 0.6 volts and a DC offset of between 0.5 and 1.5 volts.

Reader 111 then proceeds to monitor electrical signals present at distal periphery 102 of fill electrode leads 48a,52a,56a,72a of fill electrodes 48,52,56,72. Without sample liquid present within microchannel network 18, supply electrode 70 and fill electrodes 48,52,56,72 are not in electrical communication so that the electrical supply signal is not output by fill electrode leads 48a,52a,56a,72a. However, once strip 10 has been properly filled with sample liquid as discussed above, sample liquid occupies portions of microchannel network 18 between supply electrode 70 and first fill electrode 48 (in analysis channel 26) and hematocrit fill electrode 72 (in hematocrit channel 28). In this state, sample liquid places supply electrode 70 and fill electrodes 48,72 in electrical continuity and reader 111 senses the electrical supply signal at the respective contacts of 48a,72a. Reader 111 confirms that strip 10 has been properly filled with sample liquid on the basis of the sensed electrical supply signals. As the determination of the target continues, reader 111 confirms the proper filling and operation of strip 10 (e.g., the proper position and timing movement of sample liquid within microchannel network 18) by continuing to monitor the electrical supply signal at fill electrode leads 48a,72a and monitoring whether and when the electrical supply signal appears at fill electrode leads 52a, 56a and 72a as expected in response to sample liquid movement induced by the piezoelectric actuator.

One sample liquid has been applied to strip 10 and with strip 10 properly filed, sample application zone 20 (port 36) remains in gaseous communication with surrounding atmosphere 38. Therefore, the proximal gas-liquid interface of the sample liquid (i.e., the gas-liquid interface closest to and in direct gaseous communication with sample application zone 20) remains at the same gas pressure as the gas pressure of ambient atmosphere 38 surrounding reader 111 and microfluidic strip 36. Because gas bladder 60 remains sealed with respect to surrounding atmosphere 38, the gas pressure within gas bladder 60 and portions of microchannel network 18 distal to the distal gas-liquid interface of the sample liquid within analysis channel 26 (i.e., the gas-liquid interface spaced apart from sample application zone 20 by the sample liquid) is higher than the gas pressure of surrounding ambient atmosphere 38 surrounding the strip but only by an amount just sufficient to overcome the viscous drag exerted by the interaction of the sample liquid and internal walls 30 and upper and lower surfaces 12a′,14a′ of microfluidic channel network 18. Absent compression or decompression of gas bladder 60 by the piezoelectric actuator, the only source of gas pressure distal to distal liquid-gas interface 98 of the sample liquid that is in excess of the gas pressure of surrounding ambient atmosphere 38 arises from the de minimis pressure built up distal to distal liquid-gas interface 98 arising from the capillary flow of sample liquid along analysis channel 26. Any gas pressure within gas bladder 60 in excess of such de minimis excess pressure would propel the sample liquid toward sample application zone 20 (port 36). In the event of gas pressure within gas bladder 60 below such excess pressure (as occurs during decompression of gas bladder 60), the gas pressure exerted by ambient atmosphere 38 would force the liquid distally until the pressure again equalizes.

After receiving the signal that sample liquid has reached hematocrit fill electrode 72 within hematocrit channel 28, reader 111 actuates the cyan and IR LED's and opposed photodiode and determines the hematocrit of the sample liquid as described above. If the hematocrit is in excess of predetermined limits, reader 111 indicates an error via touch screen 115 and discontinues the determination of the target. Reader 111 also operates the LED's to determine whether the absolute absorption of the sample is consistent with whole blood or whether the absorption (e.g., below a specified limit) indicates that a non-whole blood sample such as plasma has been applied to the strip. If the hematocrit and absorption are within the predetermined limits, reader 111 continues with the determination.

With the determined hematocrit within the predetermined limits and distal liquid-gas interface 98 of the sample liquid having reached capillary stop 42, reader 111 actuates the flow controller to reduce compression of upper wall portion 78 overlying gas bladder 60 during a time period Tₘₒᵥ, Actuation end 121 of piezoelectric bender retracts vertically. Upper wall portion 78 (which remains under tension beneath lower surface 133 of actuation foot 127) retracts further from opposing lower substrate 14 causing the volume of gas bladder 60 to increase and reducing the gas pressure within portions of analysis channel 26 disposed distally to distal liquid-gas interface 98 of the sample liquid. With the distal gas pressure decreasing, the gas pressure exerted by surrounding ambient atmosphere 38 via port 36 on the proximal gas-liquid interface of the sample liquid overcomes any resistance created the capillary stop 42 and viscous drag of the sample liquid forcing the sample liquid distally along analysis channel 26 toward first reagent zone 44 and gas bladder 60. The piezoelectric bender actuation is calibrated to reduce the pressure within gas bladder 60 at a rate sufficient to cause portions of the sample liquid spaced apart from side walls 30 and internal surfaces 12a′,14a′ to flow at a constant rate of 1.3 mm s⁻¹ (about 96 nL s⁻¹) along the analysis channel 26 toward and into first reagent zone 44. Adjacent side walls 30 and upper and lower surfaces 12a′,14a′ of analysis channel 26, however, sample liquid flows at a lower velocity due to viscous drag forces experienced by sample liquid at these walls and surfaces. Distal liquid-gas interface 98, therefore, takes on a parabolic shape with the highest velocities in the center of analysis channel 26 spaced apart from any wall or surface and lower velocities adjacent the walls 30 and upper and lower surfaces 12a′,14a′. As distal liquid-gas interface 98 of the sample liquid passes each side cavity 46 within first reagent zone 44, the sample liquid and trapped gas within side cavity 46 form gas-liquid interface 96 at opening 68 of side cavity 46 to analysis channel 26. As the sample liquid enters first reagent zone 44, the sample liquid solubilizes lysing reagent 62 which begins to lyse cells within the sample liquid releasing target present therein.

While actuation end 121 and actuation foot 127 retract vertically, reader 111 also causes the piezoelectric actuator to impart a secondary, oscillating motion on actuation end 121 and actuation foot 127. Specifically, during a during a time period Tc, the piezoelectric actuator causes actuation end 121 to oscillate along axis a3 at an acoustic frequency, e.g., between about 500 Hz and about 2000 Hz and with a full cycle displacement of between about 7.5 μm and about 70 μm while also retracting. As actuation end 121 retracts vertically during an oscillation cycle, the pressure applied by actuation end 121 to upper surface 131 of actuation foot 127 decreases permitting actuation foot 127 to move vertically along axis a3. Upper wall portion 78 retracts vertically against lower surface 133 of actuation foot 127 driving actuation foot 127 vertically along axis a3. As actuation end 121 extends downward during an oscillation cycle, the pressure applied by actuation end 121 to upper surface 131 of actuation foot 127 increases driving actuation foot 127 downward along axis a3. Lower surface 133 of actuation foot 127 drives actuation foot 127 downward along axis a3. The oscillation of actuation end 121 causes upper wall portion 78 of gas bladder 60 to oscillate imparting pressure pulses of gas of gas bladder 60 at essentially the same oscillation frequency.

As discussed above, gas bladder 60 including contact portion 88 of outer surface 12b of upper wall portion 78 that is contacted by lower surface 133 of actuation foot 127 are spaced apart distally from portions of analysis channel 26 occupied by sample liquid (or any other liquid) during operation of microfluidic strip 10. During a determination of a target, portions of analysis channel 26 (including gas bladder 60) disposed distal to distal liquid-gas interface 98 of the sample liquid are occupied by gas, not sample liquid or any other liquid. If liquid was present in such distal portions of analysis channel 26, it would be in amounts insufficient to transmit pressure oscillations in the gas occupying gas bladder 60 to distal liquid-gas interface 98 of the sample liquid. Therefore, the effects of the oscillations of piezoelectric bender 117 upon upper wall portion 78 are transmitted to distal liquid-gas interface 98 of the sample liquid indirectly via the gas occupying gas bladder 60 and other distal portions of analysis channel 26 rather than directly to the sample by oscillations or other impacts to portions of strip 10, e.g., upper or lower substrate 12,14, occupied by sample liquid.

The gas pressure pulses impact sample liquid distal liquid-gas interface 98 causing pressure oscillations within the sample liquid. For example, the pressure oscillations within 5 gas bladder 60, peak to peak (((Pₘₐₓ−Pₘᵢₙ)/P_{avg})×100), may be between about 5% and 200%, where Pₘₐₓ is the maximum gas pressure during an oscillation cycle, Pₘᵢₙ is the minimum gas pressure within gas bladder 60 during an oscillation cycle, and P_{avg} is the average gas pressure during an oscillation cycle. The gas pressure oscillations, peak-to-peak (Pₘₐₓ−Pₘᵢₙ), may be, e.g., at least about 5 kPa and about 200 kPa or less. The gas pressure oscillations of the gas adjacent the distal liquid-gas interface of the sample liquid are at too high a frequency, e.g., an acoustic frequency, for the sample liquid to respond, during a particular oscillation, with substantial bulk movement along the longitudinal axis of the analysis channel. For example, independently of the bulk motion of the sample induced by the retraction of actuation foot 127, the location of the distal liquid-gas interface of the sample liquid may remain at essentially the same location along analysis channel 26 during a particular oscillation. Instead, the pressure oscillations within the sample liquid cause pressure oscillations within the gas trapped within side cavities 46 of first reagent zone 44 and oscillations of the gas-liquid interface at each side cavity 46. The pressure oscillations within the sample liquid and gas of side cavities 46 induce turbulent flow within the sample liquid. The turbulent flow has several effects. First, the turbulent flow enhances solubilization of lysing reagent 62 by the sample liquid. Therefore, lysing reagent 62 is more efficiently and completely solubilized than in the absence of the oscillation driven flow. Second, the flow increases the rate of bulk transport of lysing reagent 62 within the sample liquid above the diffusion limited rate of transport in the absence of the oscillation driven transport. The increased bulk transport rate causes materials within the sample liquid (e.g., solubilized lysing reagent 62 and target released by lysis of cells within the sample liquid) to sample different velocities within the flowing sample liquid so that each solubilized material experiences a similar average velocity. In the absence of the oscillation driven flow, diffusion limited transport within the sample liquid is insufficient to transport such materials to regions of different velocity on the time scale of the movement of the liquid into first reagent zone 44. Therefore, in the absence of oscillation driven flow, the sample liquid transported into first reagent zone 44, taken laterally across the width and height of the microchannel, would exhibit a range of concentrations of such materials. Because of the oscillation driven flow, however, the materials and sample liquid are more uniformly transported along first reagent zone 44 resulting in a more even concentration profile of lysing reagent 62 and lysed target across the width and height of analysis microchannel 26.

The vertical retraction and oscillation of actuation end 121 of piezoelectric bender 117 continue until distal liquid-gas interface 98 of the sample liquid reaches first fill electrode 48 at the distal terminus of first reagent zone 44. The sample liquid places supply electrode 70 in continuity with first fill electrode 48 generating the electrical supply signal at first fill electrode lead 48a indicating that the sample liquid has reached first fill electrode 48 and completely filled first reagent zone 44. The piezoelectric actuator causes actuation end 121 of piezoelectric bender 117 to cease vertical retraction ending the time period Tₘₒᵥ and maintain the then current compression of gas bladder 60. Because the volume of gas bladder 60 is no longer expanding, increasing gas pressure distal to distal liquid-gas interface 98 of the sample liquid causes the sample liquid to cease further flow along analysis channel 26. During time period Tₘₒᵥ, total volume increase of gas bladder 66 resulting from the retraction of actuation foot 127 is about the same as the total volume of analysis channel 26 displaced by the sample liquid upon advancing from analysis channel vent 40 to first fill electrode 48. Depending on the volume displaced, the total vertical retraction of upper wall portion 78 overlying gas bladder 66 is between about 15 to 40 μm along axis a3.

A predetermined time after ceasing the vertical retraction, the piezoelectric actuator causes actuation end 121 of piezoelectric bender 117 to cease oscillating ending the time period T_{osc} so that the sample liquid remains static within first reagent zone 44. The sample liquid and solubilized first reagent 62 are allowed to incubate for a period of time. During this time, lysis of target-containing cells within the sample liquid is completed.

After completion of the incubation (lysis) within first reagent zone 44, reader 111 again actuates the flow controller to further reduce compression of upper wall portion 78 overlying gas bladder 60 during a second time period Tₘₒᵥ. Actuation end 121 of piezoelectric bender retracts further vertically. Upper wall portion 78 (which remains under tension beneath lower surface 133 of actuation foot 127 retracts further from opposing lower substrate 14 causing the volume of gas bladder 60 to again increase and reducing the gas pressure within portions of analysis channel 26 disposed distally to distal liquid-gas interface 98 of the sample liquid. With the distal gas pressure decreasing, the gas pressure exerted by surrounding ambient atmosphere 38 via port 36 on the proximal gas-liquid interface of the sample liquid again overcomes any resistance created by the gas pressure distal to distal liquid-gas interface 98 of the sample liquid forcing the sample liquid distally along analysis channel 26 toward second reagent zone 50 and gas bladder 60. The piezoelectric bender actuation is calibrated to reduce the pressure within gas bladder 60 at a rate sufficient to cause portions of the sample liquid disposed in the center of the analysis channel 26 (i.e., the portions of sample liquid spaced apart from side walls 30 and internal surfaces 12a′,14a′) to flow at a constant rate of 1.3 mm s⁻¹ along the analysis channel 26 toward and into second reagent zone 50. As the sample liquid with entrained target enters second reagent zone 50, the sample liquid solubilizes labeled binding reagent 64 (with its fluorescent label) which begins to bind to the target forming first complexes.

While actuation end 121 and actuation foot 127 retract vertically, reader 111 again causes the piezoelectric actuator to impart a secondary, oscillating motion on actuation end 121 and actuation foot 127. Specifically, during a second time period T_{osc}, the piezoelectric actuator causes actuation end 121 to oscillate along axis a3 at an acoustic frequency, e.g., between about 500 Hz and about 2000 Hz and with a full cycle displacement of between about 7.5 μm and about 70 μm while also retracting. The oscillations induce the same effects described above with respect side cavities 46 as the sample liquid flows from first reagent zone 44 toward and into second reagent zone 50 with respect to the increased solubilization (e.g., the rate and efficiency of solubilization of labeled binding reagent 64 are enhanced) and the increased rate and uniformity of transport of materials within the sample liquid across the width and height of analysis channel 26. The increased bulk transport rate within the sample liquid increases the likelihood that a solubilized labeled binding reagent 64 and a target will encounter one another and bind, forming the first complex. Therefore, the extent and uniformity of the formation of first complexes between the labeled binding reagent 64 and target are higher than in the absence of oscillation driven flow.

The vertical retraction and oscillation of actuation end 121 of piezoelectric bender 117 continue until distal liquid-gas interface 98 of the sample liquid reaches second fill electrode 52 at the distal terminus of second reagent zone 50. The sample liquid places supply electrode 70 in continuity with second fill electrode 52 generating the electrical supply signal at second fill electrode lead 52a indicating that the sample liquid has reached second fill electrode 52 and completely filled second reagent zone 50. The piezoelectric actuator causes actuation end 121 of piezoelectric bender 117 to cease vertical retraction ending second time period Tₘₒᵥ and maintain the then current compression of gas bladder 60. Because the volume of gas bladder 60 is no longer expanding, increasing gas pressure distal to distal liquid-gas interface 98 of the sample liquid causes the sample liquid to cease further flow along analysis channel 26. During time period Tₘₒᵥ, total volume increase of gas bladder 66 resulting from the retraction of actuation foot 127 is about the same as the total volume of analysis channel 26 displaced by the sample liquid upon advancing from first fill electrode 48 to second fill electrode 52. Depending on the volume displaced, the total vertical retraction of upper wall portion 78 overlying gas bladder 66 is between about 15 to 40 μm along axis a3.

A predetermined time after ceasing the vertical retraction, the piezoelectric actuator causes actuation end 121 of piezoelectric bender 117 to cease oscillating ending the second time period T_{osc} so that the sample liquid remains static (except for oscillation induced flow within the sample liquid) within second reagent zone 50. The sample liquid and solubilized first reagent 62 are allowed to incubate for a period of time. During this time, the formation of first complexes between labeled binding reagent 64 and the target that began as the sample liquid first solubilized labeled binding reagent 64 is completed.

After completion of the incubation (formation of first complexes) within second reagent zone 50, reader 111 again actuates the flow controller to further reduce compression of upper wall portion 78 overlying gas bladder 60 during a third time period Tₘₒᵥ, Actuation end 121 of piezoelectric bender retracts further vertically. The piezoelectric bender actuation is calibrated to reduce the pressure within gas bladder 60 at a rate sufficient to cause distal liquid-gas interface 98 of the sample liquid disposed in the center of analysis channel 26 (i.e., the portions of sample liquid spaced apart from side walls 30 and internal surfaces 12a′,14a′) to flow at a constant rate of 1.3 mm s⁻¹ along analysis channel 26 toward and into detection zone 54. As the sample liquid with entrained first complexes enters detection zone 54, the sample liquid solubilizes the magnetic binding reagent 66 (with its magnetic particles) which begins to bind to first complex (which includes the labeled binding reagent 64 and target) forming second complexes.

While actuation end 121 and actuation foot 127 retract vertically, reader 111 again causes the piezoelectric actuator to impart a secondary, oscillating motion on actuation end 121 and actuation foot 127. Specifically, during a third time period T_{osc} the piezoelectric actuator causes actuation end 121 to oscillate along axis a3 at an acoustic frequency, e.g., between about 500 Hz and about 2000 Hz and with a full cycle displacement of between about 7.5 μm and about 70 μm while also retracting. The oscillations induce the same effects described above with respect side cavities 46 as the sample liquid flows from second reagent zone 50 toward and into detection zone 54 with respect to the increased solubilization (e.g., the rate and efficiency of solubilization of magnetic binding reagent 66 are enhanced) and the increased rate and uniformity of transport of materials within the sample liquid (e.g., the first complexes) across the width and height of analysis channel 26. The increased bulk transport rate within the sample liquid also increases the likelihood that a solubilized magnetic binding reagent 66 and a first complex and bind, forming the second complex. Therefore, the extent and uniformity of the formation of second complexes are higher than in the absence of oscillation driven flow.

The vertical retraction and oscillation of actuation end 121 of piezoelectric bender 117 continue until distal liquid-gas interface 98 of the sample liquid reaches third fill electrode 56 at the distal terminus of detection zone 54. The sample liquid places supply electrode 70 in continuity with third fill electrode 56 generating the electrical supply signal at third fill electrode lead 56a indicating that the sample liquid has reached third fill electrode 56 and completely filled detection zone 54. The piezoelectric actuator causes actuation end 121 of piezoelectric bender 117 to cease vertical retraction ending third time period Tₘₒᵥ, and maintain the then current compression of gas bladder 60. Because the volume of gas bladder 60 is no longer expanding, increasing gas pressure distal to distal liquid-gas interface 98 of the sample liquid causes the sample liquid to cease further flow along analysis channel 26. During time period Tₘₒᵥ, total volume increase of gas bladder 66 resulting from the retraction of actuation foot 127 is about the same as the total volume of analysis channel 26 displaced by the sample liquid upon advancing from second fill electrode 52 to third fill electrode 56. Depending on the volume displaced, the total vertical retraction of upper wall portion 78 overlying gas bladder 66 is between about 15 to 40 μm along axis a3.

A predetermined time after ceasing the vertical retraction, the piezoelectric actuator causes actuation end 121 of piezoelectric bender 117 to cease oscillating ending the third time period T_{osc} so that the sample liquid remains static (except for oscillation induced flow within the sample liquid) within detection zone 54. The sample liquid with entrained first complexes and the solubilized magnetic binding reagent 66 are allowed to incubate for a period of time. During this time, the formation of second complexes between the first complexes and magnetic binding reagent 66 that began as the sample liquid first solubilized magnetic binding reagent 66 is completed.

After completion of the incubation within detection zone 54, reader 111 actuates the magnetic field generator to move the magnetic field generator from the first to the second position such that the second complexes, which include the magnetic particles of second reagent 66, are forced against internal surface 14a′ of lower substrate 14 by an amount sufficient to retard motion of the second complexes in the presence of bulk motion of the sample liquid.

Once the magnetic field generator has been moved to the second position, reader 111 again actuates the flow controller to remove sample liquid, unbound (uncomplexed) labeled binding reagent 66, and other concomitant materials that might increase background signals during the detection step from detection zone 54. During a fourth time period Tₘₒᵥ piezoelectric flow control causes piezoelectric bender 117 to press lower surface 129 of actuation end 121 against upper surface 131 of actuation foot 127 increasing compression of gas bladder 60 as described in the process for initially compressing gas bladder 60 prior to the application of the sample liquid to strip 10.

Because of the sample liquid disposed within analysis channel 26 between application zone 20 (port 36) and gas bladder 60, the increased compression of gas bladder 60 (decreased volume thereof) causes the gas pressure exerted upon distal liquid-gas interface 98 of the sample liquid by the gas within gas bladder 60 to increase thereby overcoming the viscous drag of the sample liquid and the gas pressure of the surrounding ambient atmosphere acting upon the proximal gas-liquid interface of the sample liquid to drive the distal gas-liquid interface (and the proximal portions of sample liquid) out of detection zone 54 toward sample actuation port 36. The distal gas-liquid interface (and the proximal portions of sample liquid) are driven proximally to at least about the location of analysis channel vent 40.

The rate of vertical compression of gas bladder 60 by piezoelectric bender 117 is calibrated to increase the gas pressure acting upon distal liquid-gas interface 98 of the sample liquid at a rate sufficient to cause portions of the sample liquid disposed in the center of analysis channel 26 (i.e., the portions of sample liquid spaced apart from side walls 30 and internal surfaces 12a′,14a′) to flow proximally at a constant rate of 20 μm s⁻¹ (3.3 nL s⁻¹) along the analysis channel out of the detection zone 54. The flow rate in evacuating sample liquid from detection zone 54 is slower than the flow rate introducing sample liquid to detection zone 54 to reduce a tendency of second complexes to be inadvertently evacuated along with sample liquid, unbound labeled binding reagent 64 and other concomitant materials that might increase background signals during the subsequent detection step.

While actuation end 121 and actuation foot 127 compresses upper wall portion 78 overlying gas bladder 60, reader 111 causes the piezoelectric actuator to impart a secondary, oscillating motion on actuation end 121 and actuation foot 127 as discussed above. Specifically, during a fourth time period T_{osc}, the piezoelectric actuator causes actuation end 121 to oscillate along axis a3 at an acoustic frequency, e.g., between about 500 Hz and about 2000 Hz and with a full cycle displacement of between about 7.5 μm and about 70 μm while also compressing upper wall portion 78. The oscillations induce the same effects described above with respect side cavities with respect to the increased rate and efficiency of transport of materials. The extent of turbulent flow induced by the oscillations and the rate of bulk flow of the sample liquid induced by the increasing pressure are sufficiently low that the second complexes (which include magnetic binding reagent 66) remain immobilized against internal surface 14a′ of lower substrate 14 within detection zone 54. The oscillation-induced turbulent flow and bulk flow are sufficient, however, to increase the efficiency and uniformity across the height and width of the microchannel with which unbound labeled binding reagent (with its detectable label) is removed from detection zone 54.

The compression and oscillation continue until gas bladder 60 reaches the operationally fully compressed state as determined from the calibration signals stored during the initial compression of gas bladder 60 as described above. After gas bladder 60 has been re-compressed and vertical actuation of the piezoelectric actuator ceased (ending fourth time period Tₘₒᵥ), and the oscillations ceased (ending the fourth time period T_{osc}) the sample liquid (including unbound labeled binding reagent 64 and other concomitant materials) have been removed from the second reagent zone with distal liquid-gas interface 98 having been displaced proximally to about the location of capillary stop 42. The immobilized second complexes and only a thin film of residual sample liquid remains in detection zone 54. The amount of remaining second complexes is indicative of the concentration of the target in the sample liquid applied to the sample application zone (port 36). Reader 111 then actuates the optical detector to detect fluorescence from the detectable label of the second complexes. The reader determines the concentration of the target in the sample liquid based on the detected fluorescence.

Upon completion of the determination, reader 111 causes the piezoelectric actuator to completely retract actuation end 121 of piezoelectric bender 117 vertically from upper surface 129 of actuation foot 127, completely reducing compression of gas bladder 60 such that strip 10 can be removed from reader 111. Strip 10 is a single use strip and is discarded following the determination.

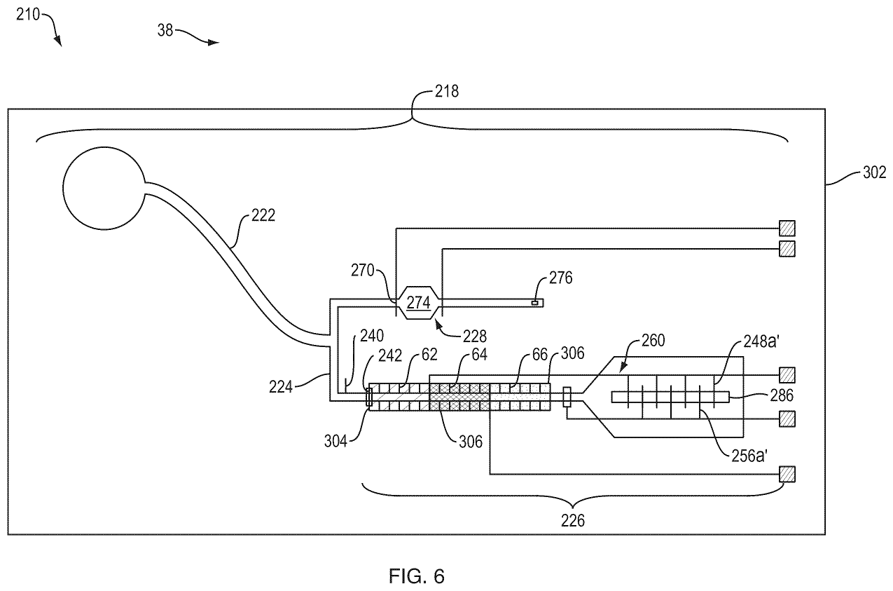

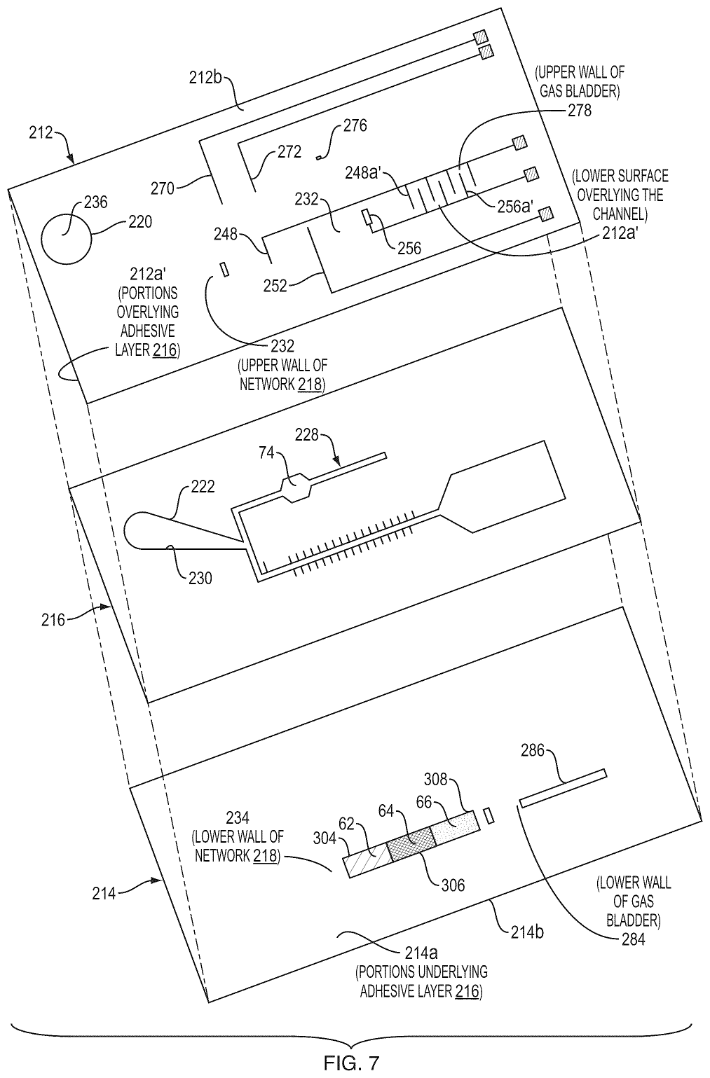

With reference to FIGS. 6 and 7, a microfluidic strip 210, is configured for use with a diagnostic reader, such as diagnostic reader 111, in the determination of the presence and/or amount of a target (e.g., a biomolecule such as a protein) present in a sample liquid applied to strip 210. Reader 111 also operates strip 210 to determine a physiochemical property, e.g., a hematocrit, of a sample liquid applied to strip 210. Reader 111 operates strip 210 as described for strip 10.

Strip 210 includes an upper substrate 212 and a lower substrate 214 each composed of 100 μm thick polyester film. A lower surface 212a of upper substrate 212 and an upper surface 214a of lower substrate 214 are adhered in opposition by an adhesive layer 216, 110 μm thick. Portions of adhesive layer 216 are absent, e.g., removed, to define a microfluidic channel network 218 between opposing surfaces 212a,214a of upper and lower substrates 212,214. Microfluidic channel network 218 has a sample application zone 220, a common supply channel 222, a branch channel 224, an analysis channel 226, and a hematocrit channel 228. Microfluidic channel network 218 has side walls 230 defined by adhesive layer 216, an upper wall 232 defined by those portions of upper substrate 212 overlying the absent portions of adhesive layer 216, and a lower wall 234 defined by those portions of lower substrate 214 underlying the absent portions of adhesive layer 216. Upper wall 232 has an inner surface 212a′ defined by those portions of surface 212a exposed by absent portions of adhesive layer 216. Lower wall 234 has an inner surface 214a′ defined by those portions of surface 214a exposed by absent portions of adhesive layer 216. Upper substrate 212 has an outer (upper) surface 212b and lower substrate 214 has an outer (lower) surface 214b.

Sample liquid applied to a port 236 of sample application zone 220 flows by capillary action along common supply channel 222 to branch channel 224 and then to analysis channel 226 and hematocrit channel 228 as described for strip 10. In strip 210, common supply channel 222 is tapered, having a width that decreases proceeding distally from port 236 to enhance the capillary force moving the liquid distally. Other than tapered common supply channel 222, the dimensions of elements of microfluidic network 218 are similar to (e.g., may be the same as) the dimensions of elements of microfluidic network 18 of strip 10. Port 236 places the channels of channel network 218 in gaseous communication with a gas, e.g., air, of surrounding ambient atmosphere 38 as described for strip 10. Gas bladder 260 is a distal terminus of microfluidic channel network 218 and is in gaseous communication with surrounding ambient atmosphere 238 via port 236, a hematocrit channel vent 276, and an analysis channel vent 240 as described for gas bladder 60 of strip 10. The portion of upper wall 232 overlying gas bladder 260 defines a gas bladder upper wall 278 and the portion of lower wall 234 underlying gas bladder 260 defines a gas bladder lower wall 284.

Hematocrit channel 228 is constructed and operates similarly to hematocrit channel 28 to facilitate the reagent-free optical determination of the hematocrit of a liquid sample of blood applied to sample application zone 220.

Analysis channel 226 is arranged and configured to facilitate the determination of the presence and/or amount of the target present in the sample liquid. Proceeding distally from branch channel 224 along a longitudinal axis of analysis channel 226, analysis channel 226 includes analysis channel vent 240, a capillary stop 242, a first reagent zone 244, a plurality of side cavities 246, a first fill electrode 248, a second reagent zone 250, a second fill electrode 252, a detection zone 254, a third fill electrode 256, a spacing channel 258, and a gas bladder 260.

As described for strip 10, the electrodes of strip 210 are disposed and configured to permit reader 111 to monitor the proper filling of strip 210 with sample liquid, the proper movement of sample liquid within strip 210 and the operation (e.g., compression state) and of gas bladder 260. Each of supply electrode 270 and fill electrodes 248,252,256,272 is disposed on internal surface 212a′ of upper wall 232 in a location that sample liquid within microchannel network 218 will contact the electrode. Each of the electrodes is connected via a respective lead to a distal periphery 302 of strip 210 to engage corresponding contacts (not shown) within reader 111. Portions of a lead 248a of first fill electrode 248 and of a lead 256a of third fill electrode 256 pass along internal surface 212a′ of a gas bladder upper wall 278 and respectively define interposed first and second interposed electrically conductive lead electrodes 248a′ and 256a′. An electrically conductive bridging contact 286 is disposed on internal surface 214a′ of gas bladder lower wall 284 and underlies lead electrodes 248a′,256a′. Bridging contact 286 and lead electrodes 248a′,256a′ operate to sense when gas bladder 260 has been fully compressed as described for gas bladder 60 of strip 10.

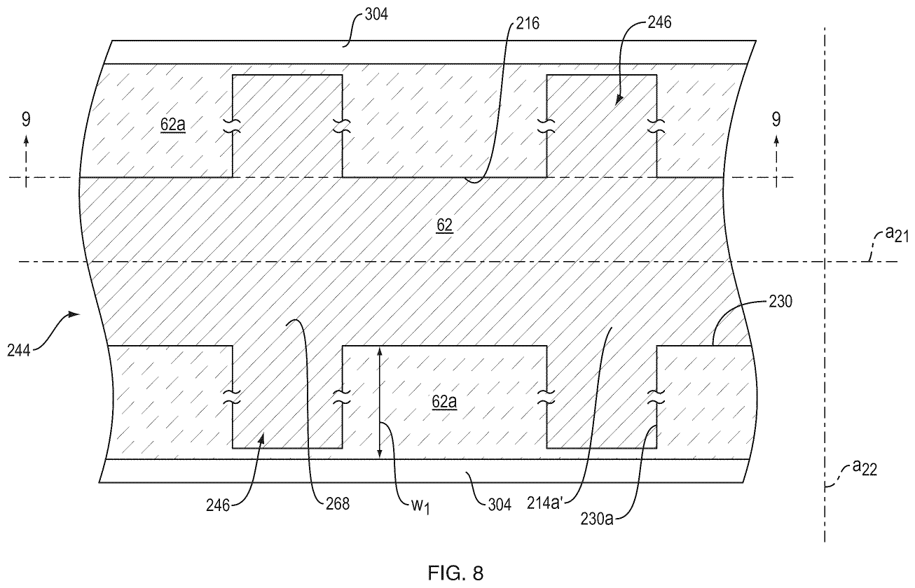

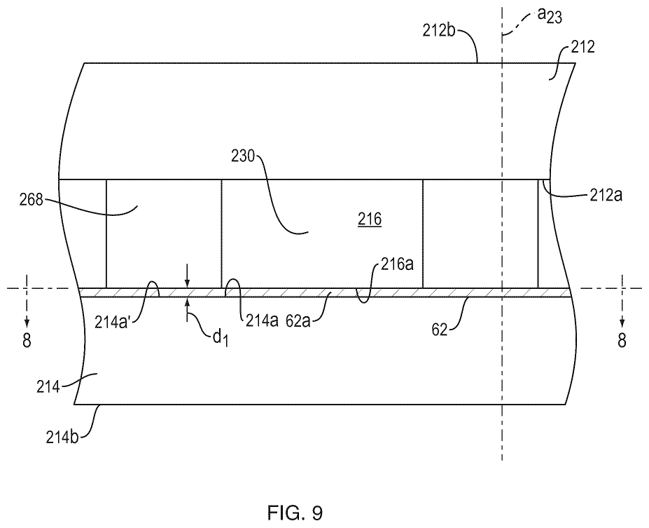

With further reference to FIGS. 8 and 9, first reagent zone 244 includes lysing reagent 62, second reagent zone 250 includes labeled binding reagent 64, and detection zone 254 includes magnetic binding reagent 66. Upper surface 214a of lower substrate 214 includes a first, second, and detection reagent deposition boundary 304,306, and 308, respectively corresponding to first reagent zone 244, second reagent zone 250, and detection zone 254 and into which reagents 62,64,66 are respectively deposited. Deposition boundaries 304,306,308 are defined by a hydrophilic material, e.g., a hydrophilic coating or layer such as an ink printed upon upper surface 214a. Each of deposition boundaries 304,306,308 has a length along a longitudinal axis a21 of analysis channel 228 approximately the same as the corresponding first, second, and detection zone 244, 250, and 254 and a width along an axis a22 generally perpendicular to longitudinal axis a21 that is greater than the width of analysis channel 228 within each respective zone 244,250,254. In the embodiment of strip 210, the width of each deposition boundary 304,306,308 is 1.5 mm and the width of analysis channel 228 is 0.8 mm.

During manufacture, each of the reagents 62,64,66 is typically deposited in a liquid state into the corresponding deposition boundary 304,306,308. Upon deposition, the reagent spreads over upper surface 214a covering most, e.g., essentially all, of the portion of upper surface 214a within each deposition boundary 304,306,308. Then, the reagents, if deposited in a liquid, rather than non-liquid state, are dried, e.g., to a lyophilized state. Once drying is complete, adhesive layer 216 is brought in contact with upper surface 214a of lower substrate 214. As discussed above, side walls 230 of microfluidic channel network 218 (including analysis channel 228) are defined by adhesive layer 216 and inner surface 214a′ of microfluidic channel network 218 (including analysis channel 226) is defined by those portions of surface 214a exposed by absent, e.g., removed, portions of adhesive layer 216. Because the width of each deposition boundary 304,306,308 is greater than the width of analysis channel 226, at least an interposed portion 62a of first reagent 62 is interposed outside of the analysis channel 226 between upper surface 214a of lower substrate 214 and the overlying adhesive layer 216. At least some of the interposed portion 62a of first reagent 62 is disposed between adjacent cavities 246 along an axis generally parallel to longitudinal axis a21 of analysis channel 226. A width w1 of interposed portion 62a taken along axis a22 between wall 230 and deposition boundary 304 depends on both the width of analysis channel 226 and deposition boundary 304 and such width may be different on one side of the channel as compared to such width on the opposite side of the channel. Independently, on either side of the channel, width w1 may be at least about 50 μm, at least about 100 μm, at least about 150 μm, or at least about 200 μm; width w1 may be about 500 μm, or less, about 400 μm or less, or about 300 μm or less.

If reagent 62 were deposited onto upper surface 214a with adhesive layer 216 already adhered to upper surface 214, the reagent might wick by capillary action through openings 268 of side cavities 246 displacing any gas therein and/or obstructing opening 268 and, therefore, the formation of a gas-liquid interface in the presence of sample (e.g., as such formation is described with respect to side cavities 46 of strip 10) and reducing or eliminating the mixing benefit afforded by side cavities 246 during oscillation of a distal liquid-gas interface of sample liquid disposed within analysis channel 226.

As seen in FIG. 9, lysing reagent 62 disposed within analysis channel 226 upon exposed surface 214a′ within first reagent zone 244 and side cavities 246 of analysis channel 226 forms a thin evenly distributed layer having a dimension d1 along an axis a23 oriented perpendicular to axes a21,a23 and a plane defined by lower substrate 214. The thin layer of reagent 62 solvates readily in the presence of sample liquid. In addition, interposed reagent 62a disposed outside analysis channel 226 upon surface 214a underlying adhesive layer 216 also forms a thin layer with dimension d1 so that a gap between a lower surface 216a of adhesive layer 216 and upper surface 214a of lower substrate 214 is sufficiently narrow to prevent sample liquid from wicking under therebetween to an extent that would cause loss of sample liquid significant enough to jeopardize either the integrity of strip 210 or the performance of an assay performed using analysis channel 226 thereof. Reagents 64,66 are similarly deposited within deposition boundaries 306,308 and form interposed portions underlying adhesive layer 316 as described for lysing reagent 62.

Once the manufacturing of strip 210 is complete, strip 210 is free of liquids as described for strip 10 and, in use, the only liquid applied to strip 210 is a sample liquid containing a target to be determined. Strip 210 is configured to not require, e.g., not configured to permit, the introduction of a liquid other than the sample liquid containing the target to be determined.

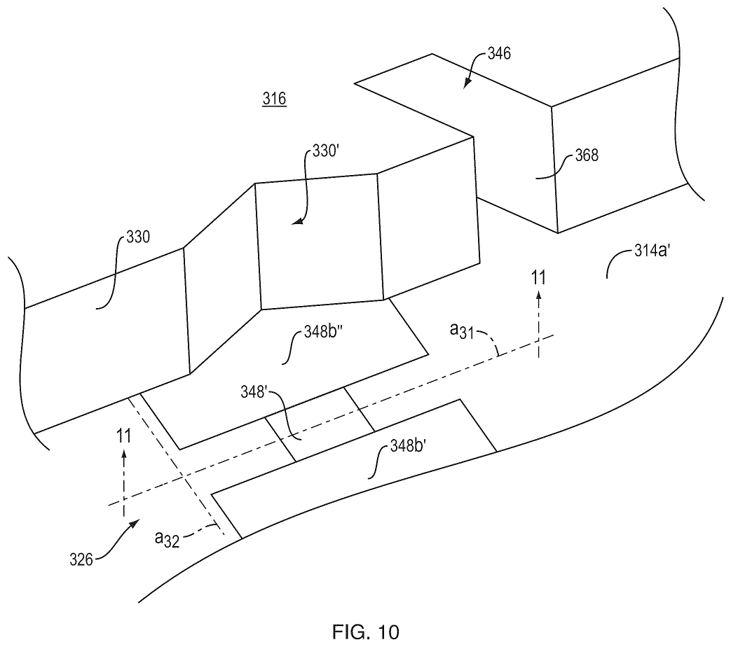

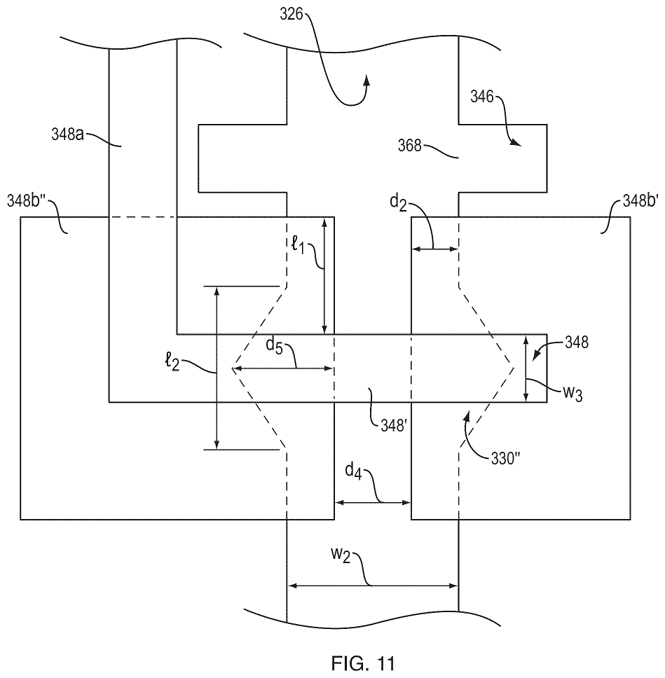

Turning now to FIGS. 10 and 11, an embodiment of an analysis channel 326 of a microfluidic strip includes a fill electrode 348 and first and second hydrophobic patches 348b′,348b″ covering all but a central portion 348′ of fill electrode 348. Central portion 348′ of fill electrode 348 remains exposed to sample liquid passing along analysis channel 326 and functions as described for fill electrodes of strips 10 and 210 to sense the presence of the liquid thereat. Each hydrophobic patch 348b′,348b″ is formed of a hydrophobic layer (e.g., a hydrophobic ink) preferably having a contact angle with deionized water determined using the sessile drop technique using a contact angle goniometer of at least about 75, at least about ° 80°, e.g., at least about 85°. Fill electrode 348 is connected by a lead 348a to a distal periphery of the microfluidic strip (not shown). Fill electrode 348 may be used in conjunction with a source electrode as described for microfluidic strips 10,210. Although FIGS. 10, 11 illustrate only a single fill electrode, analysis channel 326 may include multiple fill electrodes each have the same features as fill electrode 348 with the fill electrodes spaced apart along a longitudinal axis of the analysis channel, e.g., spaced apart by one or more reagent zones, as for analysis channels 26,226 of strips 10,210.

Analysis channel 326 is defined by walls 330 of an adhesive layer 316, a surface 314a′ of a lower substrate 314, and a surface of an upper substrate, which upper substrate, for clarity, is not shown. Wall 330 includes opposed first and second notches 330′,330″ which are generally aligned with electrode 348. Notches 330′,330″ increase the surface area of first and second hydrophobic patches 348b′,348b″ available to contact sample liquid within analysis channel 326 even if manufacturing tolerances cause slight misalignment of the various features. Analysis channel 326 also includes a plurality of side cavities 346 each having an opening 368 as described for side cavities 46,246 of strips 10,210.

Analysis channel 326 has a width w2 of about 800 μm along a transverse axis a32 perpendicular to a longitudinal axis a31 of analysis channel 326. Each hydrophobic patch 348b′,348b″ extends a distance d2 of about 280 μm from the adjacent wall 330 along transverse axis a32 and for a length 11 of 500 μm along longitudinal axis a31 on either side of fill electrode 348. Hydrophobic patches 348b′,348b″ are spaced apart from one another by a distance d4 of about 250 microns along transverse axis a32. Each notch 330′330” has a length 12 of about 1070 μm along longitudinal axis a31 and a depth d5 of about 530 μm along transverse axis a32. Electrode 348 has a width w3 of about 400 μm along longitudinal axis a31.

In practice, one or more fill electrodes 348, e.g., with hydrophobic patches 348b′,348b″ and/or notches 330′,330″, may be used with, e.g., a microfluidic strip such as strip 10,210 and a reader such as reader 111. If a sufficient amount of sample liquid is applied to the strip, and if the strip functions properly, a distal liquid-gas interface of the sample liquid moving distally along analysis channel 326 contacts central portion 348′ of fill electrode 348 and establishes continuity with a source electrode of the strip. A time-varying signal applied to the source electrode is detected by the reader at lead 348a and indicates the presence of the sample liquid at the location of fill electrode 348 within analysis channel 326. After determining that the sample liquid has contacted central portion 348′, the reader may cease movement of the sample liquid. Subsequently, the reader may reverse the movement of the sample liquid causing the sample liquid to move proximally along analysis channel 326. As the liquid-gas interface of the sample liquid moves proximally of fill central portion 348′ of fill electrode 348, hydrophobic patches 348b′,348b″ ensures the de-wetting of central portion 348′ so that a remaining film of liquid does not maintain continuity between the source electrode and central portion 348′. Accordingly, the reader determines that the time-varying signal from the source electrode is no longer detected at fill electrode 348 indicating that the sample liquid has retracted therefrom.

As discussed above, analysis channel 326 may include multiple fill electrodes with the features of fill electrode 348. The reader may continue moving the sample liquid until the distal liquid-gas interface of the sample liquid moves proximally of a second fill electrode within analysis channel 326. The second fill electrode dewets, breaking continuity between the second fill electrode and the source electrode and causing a signal indicative of such continuity to cease. The reader may then cease movement of the sample liquid, having moved the sample liquid a precise known proximal distance determined by the separation of the fill electrodes along longitudinal axis a31 within analysis channel 326. Thereafter, the reader may again reverse the direction of sample movement, causing the sample liquid to again move distally, detecting signals from the second fill electrode and then fill electrode 348 as the liquid-gas interface moves along analysis channel 326.

By detecting signals from such one or more spaced apart fill electrodes within analysis channel 326, the reader is able to precisely control and monitor the sample liquid as it repeatedly moves in a first (e.g., distal) direction and then in a second (e.g., proximal) direction. Such motion may move the sample liquid into and through and then back out of a reagent zone spaced apart by a pair of fill electrodes to facilitate reagent mobilization and/or mixing and/or binding of reagents and targets. Such motion may permit a greater volume of sample liquid to be moved through a reagent or detection zone thereby exposing reagents therein to a larger number of targets than if only a smaller volume of sample liquid were moved through the detection zone. In a zone containing magnetic binding reagents a magnet may be used to retain the reagents within a zone so that the reagents bind and concentration target present in the sample liquid at the location of the reagents. In some embodiments, binding reagents fixed, e.g., immobilized, in a zone may be used and a magnet is not used to retain the reagents while moving liquid into, through, and then back out of a zone. Sample movement may be effected by increasing or decreasing the pressure of the gas adjacent the distal liquid-gas interface of the sample liquid. The reader may also impart oscillations to the gas pressure as described for strips 10,210.

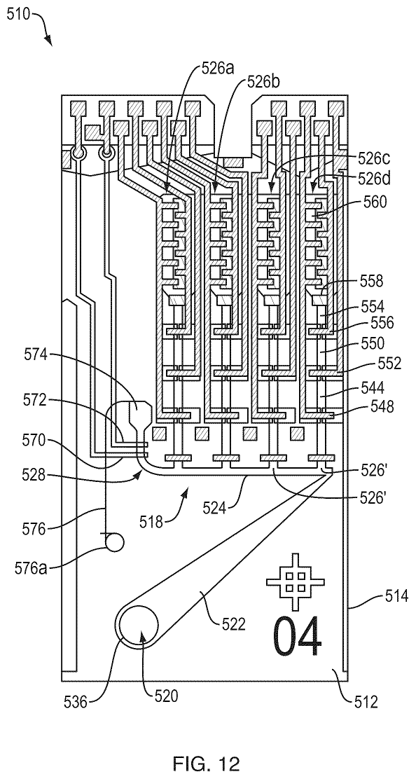

Referring now to FIG. 12, a microfluidic strip 510 includes a microfluidic channel network 518 having a sample application zone 520, a common supply channel 522, a common branch channel 524, a hematocrit channel 528 and four analysis channels 526a,526b,526c,526d. Microfluidic strip 510 is used in conjunction with a reader as described, e.g., for microfluidic strip 10, microfluidic strip 210, or analysis channel 326. Microfluidic strip 510 is formed of an upper substrate 512, a lower substrate 514 secured adhered in opposition by an adhesive layer, e.g., as described for microfluidic strips 10,210 and the microfluidic strip of analysis channel 326. Sample application zone 520 is a port 536 through upper substrate 512 as described for ports 36,236.

Hematocrit channel 528 is arranged and configured to facilitate a reagent-free optical determination of the hematocrit of a liquid sample of blood as described for hematocrit channel 28. Proceeding distally from branch channel 524, hematocrit channel 528 includes a supply electrode 570, a hematocrit fill electrode 572, a hematocrit detection zone 574, a vent channel 576 extending between hematocrit detection zone 574 and a vent 576a. Vent channel 576 has a length, between hematocrit detection zone 574 and vent 576a, of 15 mm, a height of 110 μm and a width of 150 μm. The cross sectional area of vent channel 576 is sufficiently small to substantially prevent sample liquid from entering the vent channel. Vent 576a is disposed within a proximal portion of microfluidic strip 510. In use, the proximal portion of the microfluidic strip, including vent 576a, protrudes from the reader. In the event that sample liquid is inadvertently expelled from vent 576a, the sample liquid remains external to the reader and does not contaminate the interior thereof. Sample application zone 520 and vent 576a may be the only routes by which gas may enter or exit microfluidic channel network 518.

Each analysis channel 526a,526b,526c,526d is arranged and configured to facilitate the determination of the presence and/or amount of at least one target present in a sample liquid applied to sample application zone 520. The respective target(s) determined using each analysis channel may be the same or different from the target(s) determined using the other analysis channels. Proceeding distally from common branch channel 524, each analysis channel 526a,526b,526c,526d originates at a respective proximal origin 526′ and includes a first reagent zone 544, a first fill electrode 548, a second reagent zone 550, a second fill electrode 552, a detection zone 554, a third fill electrode 556, a spacing channel 558, and a gas bladder 560. Each analysis channel has a length between proximal origin 526′ and a distal terminus of gas bladder 560 of about 20 mm.

Within each analysis channel, fill electrodes 548,552,556 include respective hydrophobic patches as described for fill electrode 348 of analysis channel 326. Within each gas bladder 560, respective leads of fill electrodes 548,556 define respective interposed lead electrodes and the gas bladder defines a corresponding bridging contact as described for gas bladder 60. The reagent zones and detection zone of each analysis channel 526a,526b,526c,526d may be configured as described for microfluidic strip 10, microfluidic strip 210, or analysis channel 326. Although not shown, each analysis channel may include side cavities as described for microfluidic strip 10, microfluidic strip 210, or analysis channel 326.

The respective proximal origin 526′ of each analysis channel connects to branch channel 524 at a different location therealong. For each of the plurality of analysis channels, the proximal origin provides the only route by which liquid and gas may enter or exit such analysis channel. The gas bladder 560 of each analysis channel defines the distal terminus thereof. In use, a distal portion of microfluidic strip 510 is received within a reader. The distal portion includes at least the gas bladder of each analysis channel and most or all of the remaining portion of each analysis channel. The reader includes a respective flow controller for each analysis channel as described for microfluidic strip 10 and microfluidic strip 210. For example, the flow controller may compress and decompress the gas bladder to either expel gas therefrom or draw gas therein. A sample liquid present in an analysis channel is moved along the analysis channel either distally toward or proximally away from the gas bladder.

In use, microfluidic strip 510 is inserted into a reader and the respective flow controller of each channel places the gas bladder of such analysis channel in the operationally fully compressed state, e.g., as described for microfluidic strip 10 and 210. As described for microfluidic strip 10 and 210, the reader calibrates the extent of compression required to fully compress upper wall portion 78 and to position each gas bladder 560 in the operationally fully compressed state and the amount of force that is required to be applied by the piezoelectric actuator in order to displace upper wall portion of each gas bladder 560. In use, the extent of displacement and amount of force required to achieve a given fluidic operation may depend on whether the upper wall of one or more other gas bladders of strip 510 is concurrently manipulated (e.g., compressed, decompressed, and/or oscillated). For example, compression of a gas bladder places the upper wall thereof under tension and other gas bladders of the strip may experience a resulting increase in tension. Therefore, the reader may acquire the calibration signals for each gas bladder in a first state in which no other gas bladder is simultaneously manipulated and/or in a second state in which one or more gas bladders of the strip is also manipulated (e.g., compressed, decompressed, and/or oscillated). For each gas bladder, the reader stores the calibration signals of the extent of displacement and amount of force required to achieve a given fluidic operation in either or both the first and second states. During operation of the strip 510, the reader can therefore operate the piezoelectric actuator of each gas bladder to manipulate such gas bladder whether or not one or more other gas bladders of the strip are concurrently manipulated.

Sample liquid is then applied to sample application zone 520. The sample liquid flows by capillary action along common supply channel 522 until reaching branch channel 524 at which point the sample liquid splits with a first portion proceeding along branch channel 524 toward hematocrit channel 528 and a second portion proceeding along branch channel 524 toward the respective proximal origin 526′ of each of analysis channels 526a,526b,526c,526d. The first portion of sample liquid proceeds to hematocrit channel 528 until the corresponding distal liquid-gas interface of the sample liquid (i.e., the liquid-gas interface of the sample liquid within hematocrit channel 528 that is spaced apart from sample application zone 520 by the aliquot of sample liquid within hematocrit channel 528, common branch channel 524 and common supply channel 522) fills hematocrit detection zone 574. As the sample liquid proceeds along hematocrit channel 528, gas is displaced from the hematocrit channel and exits microfluidic network 518 via vent channel 576 and vent 576a, but the cross sectional area of vent channel 576 substantially prevents the entry of sample liquid. The exit of gas through vent 576a permits sample liquid to fill hematocrit channel 528 by capillary action.

The second portion of sample liquid proceeds by capillary action along common branch channel 524. The sample liquid enters each of analysis channels 526a,526b,526c,526d. Because each analysis channel is sealed with respect to the ingress and egress of gas, gas pressure ahead of the sample liquid (i.e., the gas pressure distal to the distal liquid-gas interface of the sample liquid) increases and causes the distal progress of the sample liquid to cease prior to entering (i.e., proximal of) the first detection zone of each analysis channel. Subsequently, the reader operates the respective flow controller of each analysis channel to mix and/or move the sample liquid either distally or proximally along the analysis channel, e.g., as described for microfluidic strip 10, microfluidic strip 210, or analysis channel 326. The reader also operates the optical detection system, magnetic field generator, and respective flow controller to detect the one or more targets in each analysis channel.

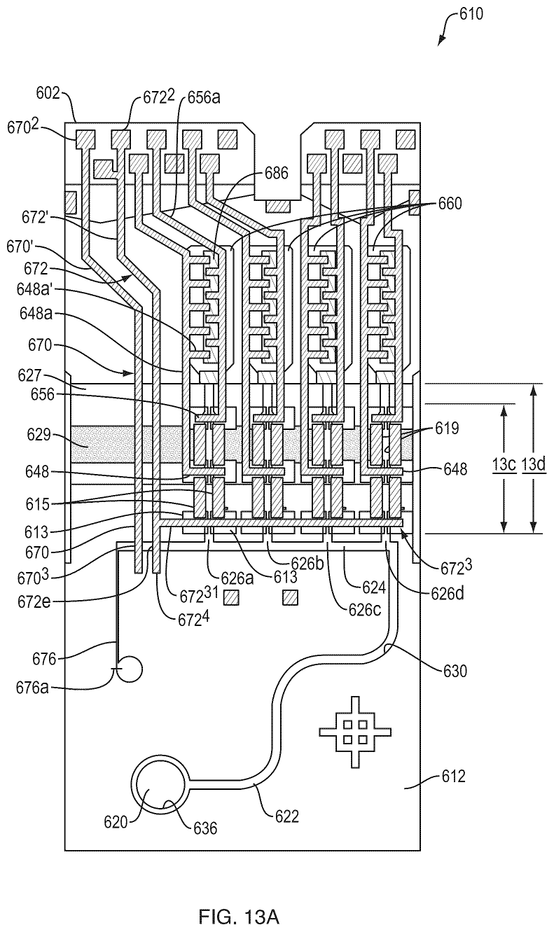

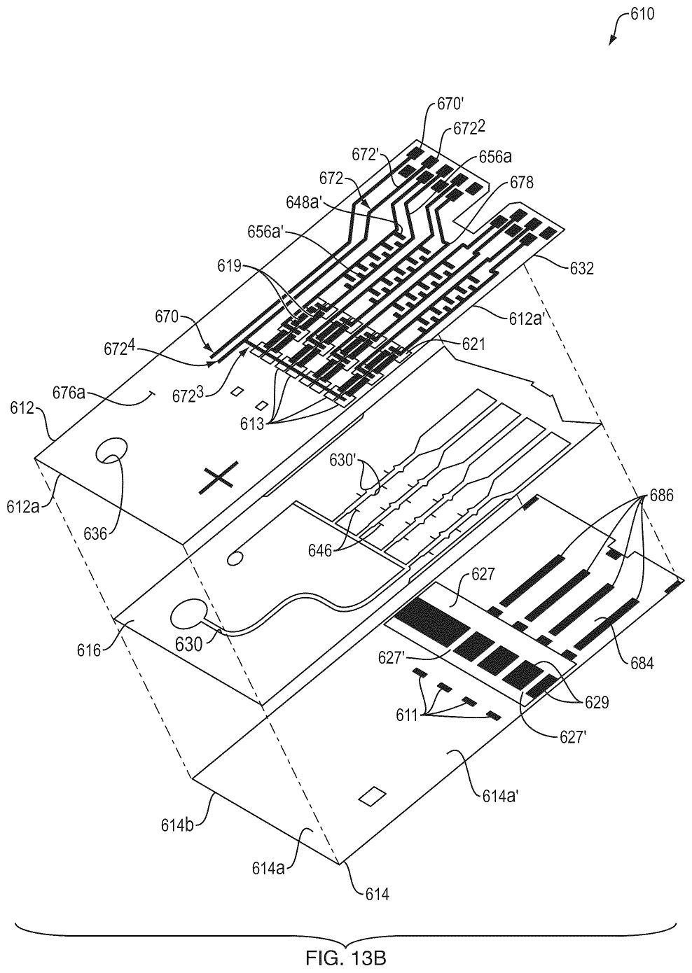

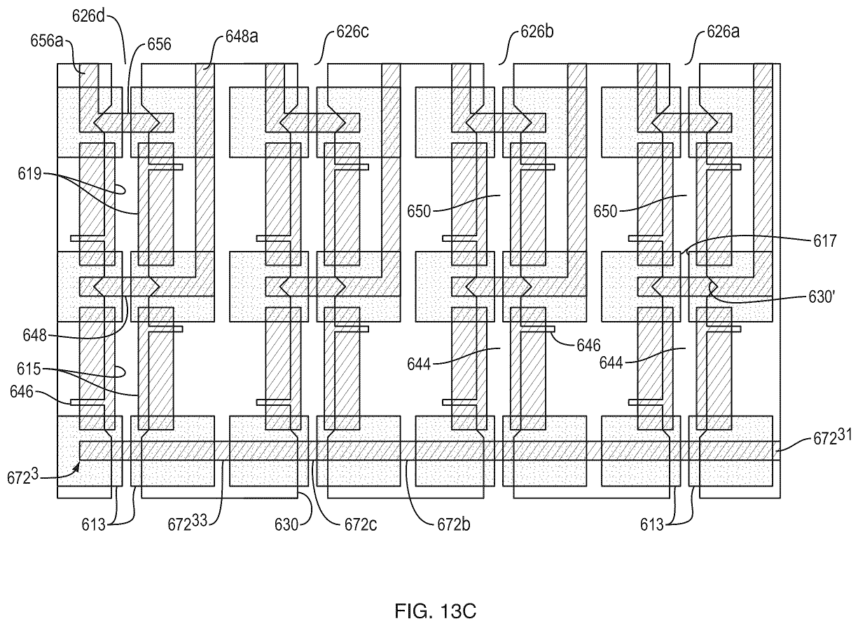

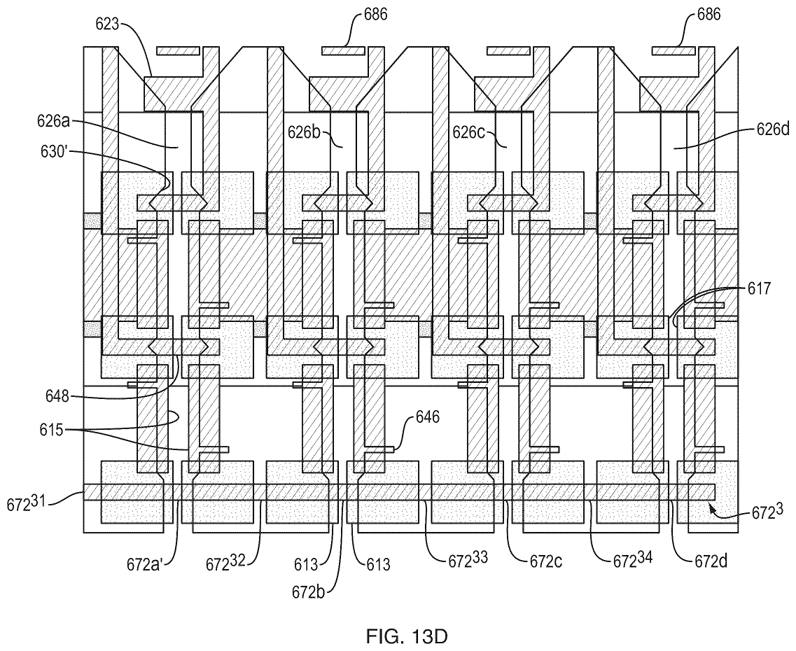

Referring now to FIGS. 13A-13D, a microfluidic strip 610 includes a microfluidic channel network having a sample application zone 620, a common supply channel 622, a common branch channel 624, and, extending therefrom, four analysis channels 626a,626b,626c,626d. Microfluidic strip 610 is formed of an upper substrate 612, a lower substrate 614 secured adhered in opposition by an adhesive layer 616, e.g., as described for microfluidic strips 10,210,510 and the microfluidic strip of analysis channel 326. Sample application zone 620 is a port 636 through upper substrate 612 as described for ports 36,236,536. Microfluidic strip 610 is used in conjunction with a reader, e.g., reader 111, and sample liquid is manipulated (e.g., mixed and/or moved within the microfluidic channel network) and targets detected as described, e.g., for microfluidic strips 10, 210, and 510, or analysis channel 326. The reader could operate the optical detection system, magnetic field generator, and respective flow controller of the reader to detect the one or more targets in each analysis channel.

The microfluidic channel network of strip 610 has side walls 630 defined by adhesive layer 616, an upper wall 632 defined by those portions of upper substrate 612 overlying the absent portions of adhesive layer 616, and a lower wall 634 defined by those portions of lower substrate 614 underlying the absent portions of adhesive layer 616. Upper wall 632 has an inner surface 612a′ defined by those portions of surface 612a exposed by absent portions of adhesive layer 616. Lower wall 634 has an inner surface 614a′ defined by those portions of surface 614a exposed by absent portions of adhesive layer 616. Upper substrate 612 has an outer (upper) surface 612b and lower substrate 614 has an outer (lower) surface 614b.

Proceeding distally from branch channel 624, each analysis channel includes a first hydrophobic stop 611, a first pair of hydrophobic patches 613, a common first fill electrode 672, a first reagent zone 644 having a first pair of reagent deposition boundaries 615, a second fill electrode 648, a second pair of hydrophobic patches 617, a second reagent zone 650 having second pair of reagent deposition boundaries 619, a third fill electrode 656, a third pair of hydrophobic patches 621, a second hydrophobic stop 623, and a gas bladder 660. Each of second and third pairs of hydrophobic patches 617,621 is associated with a respective fill electrode 648,656 and notches 630′ in sidewall 630, as described for analysis channel 326. During operation of strip 610, second reagent zone 650 is used as a detection zone.

The reagents within first and second reagent zones 644,650 are composed and configured to facilitate the determination of one or more targets and/or control reactions. For example, the reagents may be configured as the reagents of strips 10, 210, 510, analysis channel 326 or the strips of Examples 1 or 2. The reagents of each analysis channel may be configured to determine the same or different target(s) as the reagents of one or more other analysis channels of strip 610. The reagents within each reagent zone 644 are deposited on the lower surface 612a′ of upper substrate 612 between reagent boundaries 615 and the reagents within each reagent zone 650 are deposited on the lower surface 612a′ of upper substrate 612 between reagent boundaries 619. Opposing members of each pair of reagent boundaries is disposed 600 μm apart along an axis generally perpendicular to the longitudinal axis of the analysis channel. The analysis channel is 1.2 mm wide at the location of the reagent boundaries.

Strip 610 includes optical features to increase the signal to noise of fluorescence detection. For example, because opposing members of each pair of reagent boundaries 615,619 are spaced apart by a distance smaller than the distance between opposed walls 630 of the analysis channel, the reagent boundaries act as optical slits to obscure walls 630 from view by the optical detector of the reader, which directs excitation light into and detects fluorescence from the detection zone through upper substrate 612. Fluorescence that might otherwise be excited from or emitted by the adhesive of walls 630, therefore, does not reach the detector increasing the signal to noise ratio of the detection process. As another example of such features, upper surface 614a of lower substrate 614 includes an opaque diffusely reflective layer 627. Portions 627′ of reflective layer 627 form the lower internal surface 614a′ of the second reagent zone (detection layer) 650 of each analysis channel increasing the relative amount of fluorescence that is detected from fluorescence emitted by reagents therein. The reflective layer may be composed, for example, of a composition including a metallic oxide such as aluminum oxide or zinc oxide, or other material having a high reflectivity (low absorbance) of light within the bandwidth of the fluorescence to be detected. Upper surface 614a of lower surface 614 also includes opaque highly absorbent patches 629 disposed between adjacent analysis channels. Absorbant patches 629 have a high absorbance within the bandwidth of the excitation light source and optionally within the bandwidth of the fluorescence to be detected. Absorbant patches 629, therefore, reduce the amount of background fluorescence reaching the detector.

Strip 610 is configured to permit a reader to monitor and control the operation (e.g., compression state) of the respective gas bladder 660 of each analysis channel, e.g., as described herein, e.g., for strips 10, 210, 510, analysis channel 326, or the strips of Examples 1 or 2. Within each analysis channel, portions of a lead of each of two fill electrodes pass along an internal surface within the gas bladder of the analysis channel, e.g., as described for strips 10 and 210. For example, within analysis channel 626a, portions of a lead 648a of second fill electrode 648 and of a lead 656a of third fill electrode 656 pass along internal surface 612a′ of a gas bladder upper wall 678 and respectively define interposed first and second interposed electrically conductive lead electrodes 648a′ and 656a′. An electrically conductive bridging contact 686 is disposed on internal surface 614a′ of gas bladder lower wall 684 and underlies lead electrodes 648a′,656a′. Bridging contact 686 and lead electrodes 648a′,56a′ operate to sense when gas bladder 660a has been fully compressed, e.g., as described for gas bladder 60 of strip 10.

Strip 610 includes electrodes disposed and configured to permit a reader to monitor the proper filling of strip 610 with sample liquid and the proper position and movement of sample liquid within strip 610, e.g., as described herein, e.g., for strips 10, 210, 510, analysis channel 326, or the strips of Examples 1 or 2. Strip 610 includes a supply electrode 670, a common first fill electrode 672, and the respective second and third fill electrodes 648,656 of each analysis channel of strip 610 disposed on lower surface 612a of upper substrate 612 and intersects a respective channel at a location of upper wall 632 so that sample liquid within the microchannel network will contact the electrode. Each of the electrodes is connected via a respective lead to a distal periphery 602 of strip 610 to engage corresponding contacts (not shown) within the reader.

Supply electrode 670 includes a supply lead 670¹ that extends from a supply electrode contact 670² disposed at distal periphery 602 of strip 610 to a supply portion 670³ disposed within branch channel 624 so that liquid present within the branch channel 624 at the location of the supply portion will make electrical contact with supply portion 670³. When strip 610 is received by a reader, a contact (not shown) within the reader is configured to input an electrical signal to electrode contact 670², e.g., an electrical “supply” signal (e.g., a time varying signal such as a square wave or other periodic signal) as described for strip 10 and reader 111. Except for supply portion 670³, supply electrode 670 is disposed outside of the microfluidic channel network of strip 610 such that portions of supply electrode 670 other than supply portion do not make electrical contact with sample liquid present within the microfluidic network 670³.

Common first fill electrode 672 includes a common lead portion 672¹ that extends from a fill electrode contact 672² disposed at distal periphery 602 of strip 610 to a first common lead branch 672³ and a second common lead branch 672⁴. First common lead branch 672³ extends across strip 610 perpendicular to the longitudinal axis of analysis channels 626a-626d. A portion 672³¹ of first common lead branch 672³ is disposed adjacent analysis channel 626a; a portion 672³¹ of first common lead branch 672³² is disposed between analysis channels 626a and 626b; a portion 672³³ of first common lead branch 672³ is disposed between analysis channels 626b and 626c; and a portion 672³⁴ of first common lead branch 672³ is disposed between analysis channels 626c and 626d. First common lead branch 672³ includes liquid sensing portions 672a,672b,672c,672d respectively disposed within analysis channels 626a,626b,626c,626d so that sample liquid present within one of the analysis channels at the location of the liquid sensing portion therein will make electrical contact therewith. Portion 672³¹ of first common lead branch 672³ and liquid sensing portion 672a, portion 672³² of first common lead branch 672³ and liquid sensing portion 672b, portion 672³³ of first common lead branch 672³ and liquid sensing portion 672c, and portion 672³⁴ of first common lead branch 672³ and liquid sensing portion 672d for successive sensing pairs. The sensing portion of each sensing pair is disposed within a different analysis channel of the microfluidic network of strip 610.

Second common lead branch 672⁴ extends to a liquid sensing portion 672e disposed within common branch channel 624 such that liquid present at the location of liquid sending portion 672e therein will make electrical contact therewith. Except for liquid sensing portions 672a-672e, fill electrode 672 is disposed outside of the microfluidic channel network of strip 610 such that portions of fill electrode 672 other than liquid sensing portions 672a-672d do not make electrical contact with sample liquid present within the microfluidic network.

Within each reagent zone of each analysis channel of strip 610, sidewall 630 includes two offset side cavities 646, which are shaped and configured, e.g., as cavities 46, 246, 346 to facilitate mixing within each analysis channel. Each side cavity 646 is 120 μm wide, 900 μm long, and 110 μm high. Each analysis channel is 1.2 mm wide and 110 μm high. Rather than being in opposition, e.g., as shown for side cavities 46 in FIG. 3, side cavities 646 are offset from one another so that each side cavity faces an unbroken portion of wall 630 without a side cavity.

In use, liquid sample is applied to sample application zone 620 and flows by capillary action along supply channel 622 to branch channel 624, along which a first portion of the sample liquid flows by capillary action to each of the four analysis channels 626a-626d and a second portion of the sample liquid flows by capillary action along the branch channel 624 across liquid sensing portion 672e of common electrode 672, across supply portion 670³ of supply electrode 670, and ceases movement at a proximal terminus of a narrow vent channel 676, which terminates in a vent 676a. Vent channel 676 and vent 676a are sized and configured to operate as described for vent channel 576 and vent 576a. The portion of sample liquid entering each analysis channel ceases movement at the respective capillary stop 611 within each analysis channel. Within each analysis channel, the respective capillary stop 611 is positioned so that, when stopped by the capillary stop, the sample liquid contacts the respective liquid sensing portion 672a,672b,672c,672d of common electrode 672 disposed within each analysis channel.

The reader inputs an electrical supply signal, e.g., a time varying signal, e.g., as described elsewhere herein such as in Example 1 and for reader 111 and supply electrode 70 of strip 10, to supply contact 670² of supply electrode 670. The reader also determines the presence and amount (e.g., amplitude) of an electrical signal at fill electrode contact 672². If strip 610 has properly filled with sample liquid, the sample liquid establishes continuity between supply portion 670³ of supply electrode 670 and common electrode 672 along each of five pathways: (1) from supply portion 670³ and along branch channel 624 to liquid sensing portion 672e within branch channel 624 and (2)-(5) from supply portion 670³, along branch channel 624, and along the proximal portion of each analysis channel 626a-626d to the respective liquid sensing portion 672a,672b,672c,672d of common electrode 672 disposed within each analysis channel. The reader determines whether branch channel 624 and the proximal portions of each of the four analysis channels is properly filled with sample liquid based on the electrical signal determined at fill electrode contact 672² of common electrode 672 at the distal periphery 602 of strip 610. For example, if the sample liquid does not establish continuity between supply portion supply portion 670³ and one or more of liquid sensing portions 672a,672b,672c,672d, the total impedence between the supply electrode 670 and the common fill electrode 672 will be higher than if the sample liquid had established continuity between supply portion 670³ and each of the liquid sensing portions.

During subsequent manipulation of sample liquid within each analysis channel, the reader determines the presence of liquid at the respective second and third electrodes 648,656 of the analysis channel, e.g., as described for strip 10, 210, 510, or electrode 348 of analysis channel 326. Hydrophobic patches 617,621 overlie respective electrodes 648,656, leaving a central portion exposed, as described for hydrophobic patches 348b′,348b″ providing for more efficient dewetting so that the presence/absence of sample liquid can be more efficiently determined during sample manipulation as described for analysis channel 326. Sidewalls 630 of each analysis channel include notches 630′, which increase a surface area of the hydrophobic patches exposed to sample liquid as described for notches 330′ of analysis channel 326. Based on a failure to properly fill one or more analysis channels of strip 610, the reader may void (e.g., terminate) an assay performed within an improperly filled analysis channel and/or void (e.g., terminate) all assays performed using the improperly filled strip.

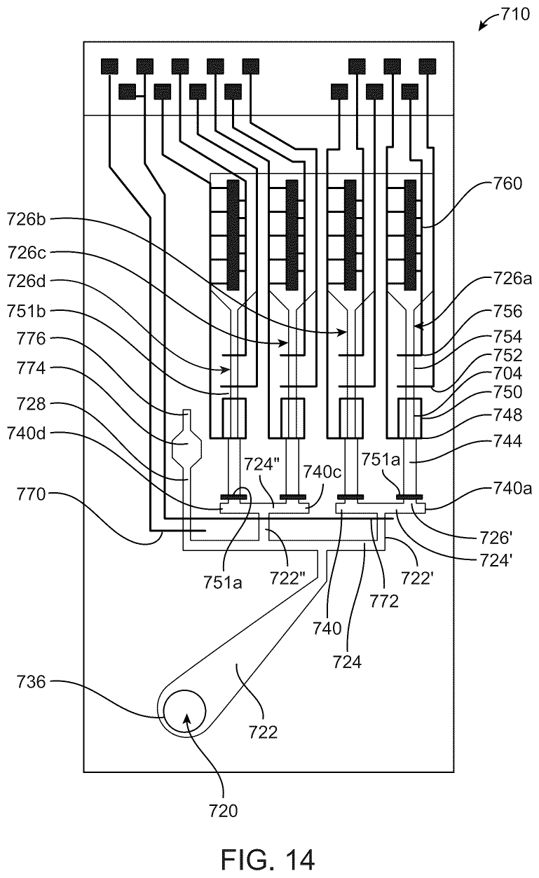

Referring now to FIG. 14, a microfluidic strip 710 includes a microfluidic channel network having a sample application zone 720 with sample application port 736, a primary common supply channel 722, a primary common branch channel 724, a hematocrit channel 728 and four analysis channels 726a,726b,726c,726d. Microfluidic strip 710 is operated by a reader, e.g., as disclosed for other microfluidic strips or analysis channels disclosed herein. Microfluidic strip 710 is formed of an upper substrate, a lower substrate secured and adhered in opposition by an adhesive layer, e.g., as disclosed for other microfluidic strips or analysis channels disclosed herein.

Primary common branch channel 724 extends to two secondary common supply channels 722′,722″ and a hematocrit channel 728. Hematocrit channel 728 includes a supply electrode 770, a common electrode 772, a hematocrit detection zone 774, and a vent 776. A reader operates the hematocrit detection 774 to determine the hematocrit of a blood sample as disclosed for other hematocrit detection zones herein.

Each secondary common supply channel 722′,722″ extends to a respective secondary common branch channel 724′,724″, each of which is fluidically connected to a respective pair of analysis channels 726a,726b and 726c,726d. Each analysis channel 726a-d is arranged and configured to prepare a respective plasma sample from a whole blood sample applied to sample application zone 720 and to determine the presence and/or amount of C-reactive protein in the plasma sample. The arrangement of the primary common branch channel and secondary common branch channels ensures that the same distance and microchannel volume is traversed by liquid sample applied to sample application port 736 and flowing to each respective analysis channel 726a,726b, 726c,726d.

Proceeding distally from a secondary common branch channel 724′,724″, each analysis channel 726a,726b,726c,726d originates at a respective proximal origin 726′ and includes a first carbon strip 751a, a first reagent zone 744, a first fill electrode 748, a second reagent zone 750, a second fill electrode 752, a second carbon strip 751b, a detection zone 754, a third fill electrode 756, a spacing channel 758, and a gas bladder 760. Each gas bladder 760 is arranged and configured as described for gas bladder 60. Each analysis channel 726a-d is associated with a respective vent 740a,740b,740c,740d disposed in secondary common branch channel 724′,724″. Each vent is in communication with the ambient atmosphere (e.g., air) surrounding strip 710.

Each first reagent zone 744 includes agglutinating reagents: 0.45 μL of a solution including 1 mg/ml phytohemagglutinin E in a trehalose-containing buffer and 0.45 μL of a solution including 1 mg/ml soybean agglutinin in a trehalose-containing buffer deposited on the underside of the upper substrate and dried. Each first reagent zone 744 has a length along the longitudinal axis thereof of 4.95 mm, a width perpendicular to the longitudinal axis of 1.2 mm, a height of 0.11 mm and a volume of 0.65 μL. Each second reagent zone 750 includes 100 nm streptavidin coated magnetic particles bound to a biotinylated first anti-CRP Fab and fluorescent particles bound to a second anti-CRP Fab applied to the upper side of the lower substrate and dried. The first and second Fabs bind CRP in a sandwich formation. Each second reagent zone 750 has a length along the longitudinal axis thereof of 3.9 mm, a width perpendicular to the longitudinal axis of 0.8 mm, a height of 0.11 mm and a volume of 0.34 μL. The reagents within each second reagent zone 750 are deposited within a respective reagent deposition boundary 704 as discussed for reagent deposition boundary 304. Each detection zone 754 includes a mix of protein-blocking components applied to the underside of the upper substrate and dried. Each detection zone 754 has a length along the longitudinal axis thereof of 2 mm, a width perpendicular to the longitudinal axis of 0.8 mm, a height of 0.11 mm and a volume of 0.17 μL.

Each carbon strip 751a,b is formed of printed hydrophobic carbon 500 μm long along the longitudinal axis of each analysis channel 726, about 5 μm in height, and having an arithmetic roughness of Sa −0.8. Each reagent deposition boundary 704 is formed of printed hydrophobic carbon having the same length (width) and height as the carbon strips.

Strip 710 may be operated as follows. The strip is inserted into a reader and the gas bladder for each analysis channel is moved to the operationally fully compressed state, e.g., as disclosed for gas bladder 60 of strip 10. The reader operates a magnetic field generator as disclosed for reader 111. A whole blood sample is then applied to application port 736 of application zone 720. The whole blood sample flows by capillary action along common supply channel 722 and primary common branch channel 724, from which channel 724 a first portion of the whole blood sample flows by capillary action into hematocrit detection zone 774 and respective second portions of the whole blood sample flow by capillary action into secondary common branch channels 724′,724″ until a respective distal liquid-gas interface of each respective second portion of whole blood reaches the respective proximal origin 726′ of an analysis channel. Each respective vent 740a-d and carbon strip 751 act as a capillary stop causing the capillary flow of whole blood to stop with the respective distal liquid-gas interface at the proximal origin of the analysis channel. The presence of the whole blood in each secondary common supply channel is determined using supply electrode 770 and common electrode 772, e.g., as disclosed for common electrode 672.

The reader then actuates the flow controller to reduce the pressure of the gas of the respective distal liquid-gas interface of each whole blood sample thereby drawing each sample along the respective analysis channel until whole blood fills each first reagent zone 744 and the respective distal liquid-gas interface of the whole blood sample reaches second fill electrode 752 at which point the actuator ceases, causing the whole blood sample to stop flowing. Whole blood within each first reagent zone mobilizes and combines with the agglutinating reagents therein. After a brief incubation, e.g., between about 5 and 20 seconds, the actuator begins to oscillate the pressure of the respective gas of the distal liquid-gas interface in each analysis channel causing the whole blood sample to oscillate proximally and distally within each channel. The distal liquid-gas interface of each whole blood sample is oscillated about a distance of about the length of the first reagent zone, e.g., ±about 5 mm and through a volume of about the volume of the first reagent zone, e.g., ±about 0.65 μL. The cycle time of each complete oscillation is between about 1 and 5 seconds, e.g., about 2 seconds per oscillation. The rate of motion of the distal liquid-gas interface along each analysis channel is between about 1 and 10 mm per second, e.g., about 5 mm per second. The oscillations further combine the whole blood sample in each first reagent zone and the agglutinating reagents therein. The number of oscillations is between about 3 and 20, e.g., about 10.

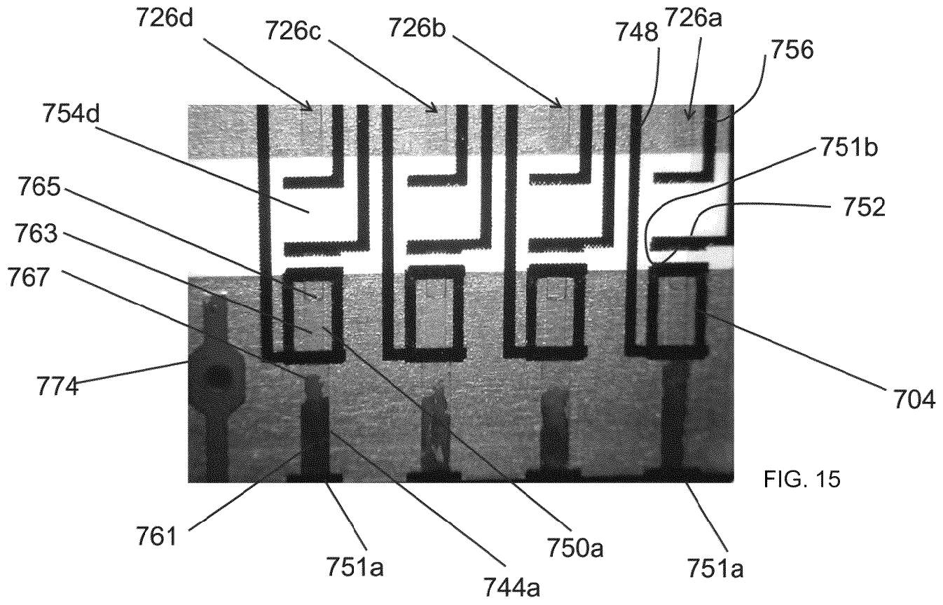

At the completion of the oscillations, the actuator stops flowing the whole blood samples combined with the agglutinating reagents with the respective distal liquid-gas interface of sample in each respective analysis channel at about the location of first carbon strip 751a therein. The actuator then begins to reduce the pressure of the gas of the distal liquid-gas interface causing each whole blood sample with combined agglutinating reagents to move distally within each analysis channel toward the respective gas bladder 760 thereof. The rate of motion of the distal liquid-gas interface along each analysis channel is between about 0.05 and 2.5 mm per second, e.g., about 0.2 mm per second. As each whole blood sample with combined agglutinating reagents moves distally within a respective analysis channel, plasma moves at a higher velocity than the red blood cells. With reference to FIG. 15, each sample separates into a red blood cell portion 761 and a plasma portion 763 having a distal liquid-gas interface 765. The red blood cell portion 761 and the plasma portion 763 are connected by a liquid-liquid interface 767. The actuator continues moving the red blood cell portion 761 and plasma portion 763 until plasma portion 763 fills second reagent zone 750 and distal liquid-gas interface 765 contacts second fill electrode 752, at which time the actuator ceases movement. The distal liquid-gas interface 765 of plasma portion 763 is spaced apart from the ambient atmosphere surrounding strip 710 by at least plasma portion 763 and red blood cell portion 761.

The respective plasma portion within each second reagent zone mobilizes and combines with the first anti-CRP Fab and second anti-CRP Fab reagents disposed therein. After a brief incubation, e.g., between about 5 and 20 seconds, the actuator begins to oscillate the pressure of the respective gas of the distal liquid-gas interface in each analysis channel causing the red blood cell portion 761 and the plasma portion 763 to oscillate proximally and distally within each channel. The distal liquid-gas interface 767 of each plasma portion is oscillated about a distance of about one-half of the length of the second reagent zone, e.g., about ±2 mm and through a volume of about one half of the volume of the second reagent zone, e.g., ±0.325 μL. The cycle time of each complete oscillation is between about 1 and 5 seconds, e.g., about 2 seconds per oscillation. The number of oscillations is between about 2 and 10, e.g., about 3. During the incubation and oscillations, the plasma portion mobilizes the first anti-CRP Fab and second anti-CRP Fab reagents disposed within each second reagent zone 752.

At the completion of the incubation and oscillations within each second reagent zone, the actuator then begins to reduce the pressure of the gas of each distal liquid-gas interface 767 causing the red blood cell portion and plasma portion to move distally within each analysis channel toward the respective gas bladder 760 thereof until the respective distal liquid-gas interface of the plasma portion 763 contacts third fill electrode 756 in the respective analysis channel. The reader operates the magnetic field generator, optical detector, and flow actuator to capture the magnetic particle reagent within each detection zone, remove plasma containing unbound detectable label, and measure the amount of detectable label retained in the detection zone as disclosed for strip 10 and reader 111.

The various embodiments disclosed herein are exemplary and may be modified. In embodiments, for example, a microfluidic strip has a different configuration and/or construction. A microfluidic strip may be formed of fewer or more than three layers (e.g., substrates). For example, a strip may be formed by two layers secured, e.g., adhered, together with a microfluidic channel network formed (e.g., by stamping, etching or laser ablation) in the inner surface of one or both layers. As another example, microfluidic strip may be formed of more than three layers with a microfluidic channel network or portions thereof disposed between each of multiple opposed layers and with connections between layers passing through one or more of the layers. The microfluidic strip may be formed of polymers other than polyester, with suitable polymers including, e.g., polydimethylsiloxane (PDMS) elastomers and thermoplastics. The microfluidic strip may be formed of non-polymeric materials or of layers of different materials, e.g., with one or more rigid layers formed of, e.g., polymer, quartz or silicon, and one or more flexible layers formed, e.g., of a polymer.

In some embodiments, e.g., using optical detection, one or more layers of a strip overlying and/or underlying a detection zone may exhibit a high transmittance of light at a wavelength range of optical irradiation (e.g., fluorescence excitation) into the detection zone and/or a wavelength range of optical emission (e.g., fluorescence emission, scattering, or transmitted irradiated light) from the sample within the detection zone. In embodiments, fluorescence is excited by excitation light passing through a layer of the strip (e.g., an overlying layer) into the detection zone and fluorescence emitted from within the detection zone is collected after passing through a layer (e.g., the same layer through which the excitation light passed). The strip may include a non-absorptive layer opposing the layer through which excitation and emitted light passed. The layer may be placed, e.g., underlying the layer that defines the floor or top of the microchannels of the strip. Alternatively, a surface of the non-absorptive layer may define a floor or top of the channel within at least a portion, e.g., all, of the detection zone. By non-absorptive, it is meant that the layer has a low absorbance at least with respect to light within the range of light emitted by the sample. For example, for fluorescence emission in the visible spectrum, a strip may include a layer with a generally white appearance when illuminated with generally colorless light (e.g., sunlight). The non-absorptive layer may have a surface roughness of about the same dimensions as the wavelength of emitted light (e.g., between about 200 nm and about 2500 nm) so that the surface is matt or roughened rather than having a mirror-like finish.

Layers of a microfluidic strip may be secured with respect to one another by techniques other than by an adhesive layer. For example, layers may be secured with respect to one another by other indirect bonding techniques using an additional material(s) to perform the securing of layers, such as epoxy, adhesive tape, or other chemical reagents. Thermoplastic bonding uses an intermediate layer, such as metal or a chemical reagent and may be performed with different methods, such as adhesive bonding or microwave bonding. As other examples, layers may be secured with respect to one another by direct bonding techniques including thermal fusion bonding, ultrasonic welding, surface modification, solvent bonding without the use of, or with only minimal use of, any additional materials added to the interface between layers. Further examples include anodic bonding, polymer-substrate bonding, low-temperature bonding, or high-temperature bonding.

A microfluidic strip may have microfluidic channel networks different from microfluidic channel network 18, 218, 518, or the microfluidic network of strip 610. For example, a microfluidic channel network may include fewer, or more, channels or reagent and/or detection zones than described for channel network 18, 218, 518, or the microfluidic network of strip 610. The dimensions of a microfluidic channel network, e.g., the dimensions of various channels, reagent zones, detection zones, and/or gas bladder may be different from microfluidic channel network 18, 218, 518, or the microfluidic network of strip 610. The dimensions of the microfluidic network, including channels thereof, typically permit sample liquids to flow by capillary action therein and typically have volumes on the order of pL to μL, e.g., between about 3 μL and 10 μL. The reagents may be different from those described for first and second reagent zones and detection zones of strip 10, 210, 510, or 610. In embodiments, a hematocrit determination channel is disposed in series with the analysis channel rather than disposed within a separate channel as described for strip 10, 210, or 510. Typically, such in-series hematocrit determination channel is disposed proximally of the analysis channel so that blood passes through the hematocrit detection zone before reaching the reagent zone(s) of the analysis channel. The sample application zone, e.g., port, of a microfluidic strip may include a filter or membrane configured to exclude a portion of an applied sample from entering the microfluidic network of the microfluidic strip. For example, the filter or membrane may be a plasma separation membrane configured to permit plasma to enter the microfluidic network upon the application of blood thereto.

Side cavities of a microchannel, e.g., an analysis channel, of a microfluidic strip typically have a longitudinal axis oriented at a non-zero angle with respect to a longitudinal axis of the microchannel at the location of the opening of the side cavity to the microchannel. For example, each of one or more side cavities of the microchannel may have a longitudinal axis having an angle of at least about 20°, at least about 35°, at least about 45°, at least about 67.5°, or at least about 85° with respect to the longitudinal axis of the microchannel at the location of the opening of the side cavity to the microchannel. Each of one or more side cavities of the microchannel may have a longitudinal axis having an angle of about 160° or less, about 145° or less, about 135° or less, or about 120° or less with respect to the longitudinal axis of the microchannel at the location of the opening of the side cavity to the microchannel. For example, the longitudinal axes of each of a plurality of side cavities and the longitudinal axis of the microchannel at the location of such side cavity may be generally perpendicular to one another.

Side cavities of the microchannel may be arranged and configured such that the net effect of oscillating the gas pressure, e.g., oscillating the gas pressure at acoustic frequencies, at the gas-liquid interfaces as disclosed herein induces little to no force, e.g., essentially no force, tending to propel the liquid along the longitudinal axis of the capillary channel. In embodiments, the net effect of the oscillations of a plurality of side cavities may be insufficient to propel the liquid at a velocity along the longitudinal axis of the capillary channel of greater than about 125 μm s⁻¹, greater than about 62.5 μm s⁻¹, greater than about 30 μm s⁻¹, greater than about 25 μm s⁻¹, greater than about 15 μm s⁻¹, greater than about 7.5 μm s⁻¹, or greater than about 0 μm s⁻¹. For example, when subjected to oscillation as disclosed herein, the net effect of a plurality of side cavities arranged within a reagent or detection zone may induce insufficient force to propel the liquid out of such reagent or detection zone during a time period sufficient to mobilize a dried reagent present therein, mix a sample liquid and a reagent disposed therein, and/or incubate the reaction between a target and a reagent disposed therein. In embodiments, a longitudinal axis of each of a first set of side cavities within a reagent or detection zone may be oriented at a first angle with respect to the longitudinal axis of the microchannel within the reagent or detection zone and a longitudinal axis of each of a second set of side cavities within a reagent or detection zone may be oriented at a second angle with respect to the longitudinal axis of the microchannel within the reagent or detection zone, where the first and second angle oppose one another. For example, openings of each of the first set of side cavities may face generally proximally within the microchannel and openings of each of the second set of side cavities may face generally distally within the microchannel. Alternatively, or in combination, the longitudinal axes of each of a plurality of side cavities and the longitudinal axis of the microchannel at the location of such side cavity within the reagent or detection zone may be generally perpendicular to one another. In such embodiments, bulk motion of the liquid along a longitudinal axis of the capillary may be induced, e.g., by increasing or decreasing a gas pressure adjacent a distal liquid-gas interface of the liquid, which step(s) may be performed sequentially with, and/or simultaneously with the oscillations of the gas pressure.

A microfluidic strip may have a different disposition of elements, e.g., reagents, reagent deposition boundaries, vents, capillary stops, leads, electrodes, and/or bridging contact, than strip 10,210,510,610 or the strip of analysis channel 326. For example, some, or all of elements described as being on a lower surface may instead be disposed on an upper surface or side wall of a microfluidic channel network; some or all of elements described as being on an upper surface may instead be disposed on a lower surface or side wall of a microfluidic channel network.

Microfluidic channel networks 18,218, 518, and the microfluidic network of strip 610 are in communication with surrounding ambient atmosphere 38 via sample application zone 20,220,520,620 (port 36,236,536,636). Other configurations are possible. For example, a sample introduction zone (port) of a microfluidic channel network may be fitted with a cap being of sufficient volume or being configured with a variable volume to permit sample liquid to flow and/or move within the microfluidic channel network without inhibition by gas pressure buildup or decrease proximal to the sample liquid.

A microfluidic strip may include multiple analysis channels, e.g., multiple analysis channels each connected to a common branch channel and configured as analysis channel 26, analysis channel 226, analysis channel 326, analysis channels 526a,526b,526c,526d or analysis channels 626a,626b,626c,626d. Each analysis channel may have its own gas bladder, each independently actuable of the other gas bladders to permit independent control over the manipulation (e.g., mixing by oscillation and/or flow) of liquid within the corresponding analysis channel. A reader may be configured with multiple flow controllers, such as flow controllers configured as the flow controller of reader 111 each including an actuator, e.g., a piezoelectric actuator such as a piezoelectric bender, each configured to independently control the volume and/or oscillation of a corresponding gas bladder. In use, each of one or more actuators may be oscillated out-of-phase (e.g., in antiphase) with the oscillation of one or more other actuators of the reader. For example, as one or more first actuator(s) compresses the respective gas bladder(s) of one or more first analysis channel(s) of a microfluidic strip during an oscillation cycle, one or more second actuator(s) simultaneously retract from the respective gas bladder(s) (allowing expansion thereof) of one or more second analysis channel(s) of the microfluidic strip during an oscillation cycle. Therefore, as the first actuator(s) increases the gas pressure(s) distal to a liquid-gas interface of a sample liquid present in the one or more first analysis channel(s) the second actuator(s) decreases the gas pressure distal to a liquid-gas interface of a sample liquid present in the one or more second analysis channel(s). The out-of-phase oscillation can reduce sound emitted by the system resulting in quieter operation.

Each analysis channel of a microfluidic strip may have a function different from the function of other analysis channels of the microfluidic strip, e.g., determination of a different target or property of the sample. Multiple targets or sample properties may be determined within a single analysis channel. A single source electrode may be used to introduce an electrical signal into a microfluidic channel network with the signal detected by fill electrodes in each of multiple respective different analysis channels. Exemplary microfluidic strip and channel configurations are disclosed in, e.g., the aforementioned '946 application.

The actuator may impart gas pulses differently from the actuator of reader 111. For example, an actuator may impart gas pulses by compressing a lower wall of a microfluidic strip as an alternative or in addition to an upper wall of a microfluidic strip. An actuator may utilize an oscillating piston or membrane in gaseous communication with a liquid-gas interface of sample liquid. A reader and strip may be configured to place a portion of a microfluidic channel network of the strip in gaseous communication with a gas within the reader in order to apply gas pressure and/or oscillations to a liquid-gas interface of liquid within the microfluidic channel network of the strip. A strip may be configured to apply gas pressure and/or oscillations to a liquid-gas interface adjacent a proximal gas-liquid interface or a lateral gas-liquid interface adjacent a side wall of a channel.

A microfluidic strip may be configured to permit the introduction of one or more additional liquids other than a sample liquid containing a target. For example, a microfluidic strip may be configured to permit the introduction of a reagent liquid, such as a buffer, via the same sample introduction zone as used to introduce the sample liquid or via a separate liquid introduction zone. As an alternative, or in combination, a sample strip may be configured and manufactured to include a liquid reagent, which may be contained within a hermetically sealed chamber of the microfluidic strip.

Implementation of oscillations during a time T_{sc} may be different than described for the operation of diagnostic system 101. For example, oscillations may occur during none, or only a portion of a time period Tₘₒᵥ in which liquid is flowing within a particular portion (e.g., a reagent zone) of microfluidic channel network 18. The frequency and/or peak-to-peak displacement of the gas bladder wall induced by the oscillations may be modified during a time T_{osc} of a particular sequence of oscillations. The frequency and/or peak-to-peak displacement of the gas bladder wall induced by the of oscillations may be lower or greater than the frequency and/or peak-to-peak displacement of the gas bladder wall induced oscillations described for diagnostic system 101. For example, the frequency and/or peak-to-peak displacement of the gas bladder wall induced by the oscillations may be implemented as a function of a rate of change in gas pressure used to move liquid within a microfluidic channel network, e.g., a lower frequency and/or peak-to-peak displacement of the gas bladder wall induced by the oscillations than described for diagnostic system 101 may be used when expelling sample liquid from a detection zone so as to reduce the likelihood of inadvertent expulsion of bound target. As another example, the distance traveled by the gas bladder wall (peak-to-peak) of oscillations of the gas bladder wall and/or the actuation member (e.g., actuation foot) driving the oscillations of the gas bladder wall during time T_{osc} may be at least about 7.5 μm, at least about 12.5 μm, or at least about 15 μm. The peak-to-peak displacement of oscillations of the gas bladder wall and/or the actuation member (e.g., actuation foot) driving the oscillations of the gas bladder wall during time T_{osc} may be about 60 μm or less, about 50 μm or less, about 40 μm or less, about 17.5 μm or less, about 15 μm or less, about 12.5 μm or less, or about 10 μm or less.

The oscillation may be performed by oscillating at least a portion of gas bladder at a frequency that is at or substantially the same as a resonance frequency ωr of the wall of the gas bladder. The resonance frequency ωr of the gas bladder wall may vary as, e.g., a function of the tension of the wall of the gas bladder and/or the composition and structure of the wall. For example, the oscillation frequency may increase with increasing tension of the wall and decrease with decreasing tension of the gas bladder wall. The resonance frequency ωr of the wall may be determined by using an actuator, such as a piezoelectric actuator, e.g., a piezoelectric bender, to oscillate the gas bladder wall at a frequency ω 1 and then ceasing to drive the oscillation of the wall at the frequency ω 1. Once the wall is no longer being driven by the actuator, the wall, which is under tension, continues to move with the magnitude of such movement related to the related to the efficiency of the oscillations driven by the actuator at frequency ω1. The magnitude of motion can be determined, for example, by use of a displacement transducer which converts the movement of the wall to an electrical signal. The displacement transducer may be the actuator used to oscillate the wall at frequency ω1, the mode of operation of which is reversed from that of the actuator to that of a displacement transducer. Upon determining the magnitude of the motion of the wall in response to the wall having been oscillated at frequency ω1, the system again uses the actuator to oscillate the wall, now at a different frequency ω2. For example, the system may reverse the operation of the displacement transducer to again act as an actuator. The system then repeats the steps of ceasing to drive the oscillation of the wall, determining the magnitude of oscillation, and oscillating the wall at a different frequency. The determined magnitude is greatest when the oscillation frequency corresponds to the resonance frequency ωr. Once the resonance frequency ωr is determined, the system continues to drive oscillations of the wall at resonance frequency ωr or a frequency substantially similar thereto. To ensure that the oscillations remain at or near frequency ωr, the system may, after driving the oscillation for a number of cycles at frequency ωr or a frequency near thereto, perform the steps of ceasing to drive the oscillation of the wall at frequency ωr, determining the magnitude of oscillation, and oscillating the wall at a different frequency ωr′, where ωr′ is a frequency near (e.g., without about 3% to 10%) of frequency ωr. Depending on whether the determined magnitude of wall oscillation is greater or smaller than the oscillation at frequency (or, the system may continue the steps of ceasing to drive the oscillation of the wall, determining the magnitude of oscillation, and oscillating the wall at a different frequency to maintain the oscillation at a frequency of or about the same as the resonance frequency of the wall. For example, the steps of ceasing, determining, and then driving the oscillation of the wall may be repeated at least once within every Nth oscillation wherein N is about 500 or less, about 250 or less, about 125 or less, or about 75 or less. As an alternative to, or in combination, the reader may use a non-contact technique, such as an optical or acoustic technique, to determine the magnitude of movement of wall of the gas bladder.

Implementation of motion of liquid during a time Tₘₒᵥ may be different than described for the operation of diagnostic system 101. For example, the velocity of the liquid may be varied during time Tₘₒᵥ. As a particular example, during a step of evacuating sample liquid from a detection or reagent zone, but retaining a particular material (e.g., a bound target) within the detection or reagent zone, the liquid may be propelled at a first, reduced velocity until the sample liquid has evacuated the detection or reagent zone and then at a second, higher velocity to expedite preparation of the strip for a subsequent liquid manipulation or detection step. As an alternative, or in combination with, to the use of gas pressure to induce bulk motion of liquids or materials along a capillary channel, other techniques may be used such as electroosmotic or other electrokinetic techniques.

As discussed above with respect to strip 10 and system 101, sample liquid movement induced by vertical retraction and oscillation of actuation end 121 of piezoelectric bender 117 continues until distal liquid-gas interface 98 of the sample liquid reaches third fill electrode 56 at the distal terminus of detection zone 54. In embodiments, sample liquid is moved a greater distance beyond a detection zone (or other zone including reagents therein) of a strip so that biding reagents disposed within the detection zone (or other zone including reagents therein) are exposed to a volume of sample liquid greater than the volume of the detection zone (or other zone including reagents therein), e.g., at least about 1.5×, at least about 2×, at least about 3×, at least about 5×, or at least about 7.5× greater than the volume of the detection zone or such other zone. In some embodiments, a length of channel interposed between the detection zone and a gas bladder is increased as compared to the embodiment of strip 10. A fill electrode disposed within a distal portion of such longer interposed channel may be used to sense the position of the sample liquid-gas interface as discussed above. Alternatively, or in addition to such longer interposed channel, sample liquid may be drawn into the gas bladder so that the volume of the gas bladder can be used to increase the volume of sample liquid that is moved through the detection zone (or other zone including reagents therein). Sample liquid moved distally into and through the detection zone (or other zone including reagents therein) may be moved back proximally into and through the detection zone as described above, e.g., with respect to analysis channel 326 and FIGS. 10 and 11. This process may be repeated multiple times, e.g., at least 2×, at least about 3×, at least about 5× or at least about 10× thereby increasing the number of opportunities for binding reagents disposed within the detection zone (or other zone including reagents therein) to encounter and bind to targets in the sample liquid. During periods when sample is moved into (in either the distal or proximal direction) a magnetic field generator (e.g., as described above) may be used to retain magnetic binding reagents within the detection zone (or other zone including reagents therein). During a sequence of moving sample liquid into, through, and back into and through the detection zone (or other zone including reagents therein), movement of the sample liquid may be paused to permit incubation of binding reagents therein with targets present in the same sample volume. During such incubation time, a magnetic field applied to a zone (if used) may be turned off or moved to a location or position that does not exert a force sufficient to retain magnetic particles within the zone. Accordingly, magnetic binding reagent particles may diffuse more freely permitting even more encounters with target present with the magnetic binding reagent and the accumulation of a larger number of target molecules on the magnetic binding reagent. Upon completion of the incubation time, the magnetic field is once again applied to retain the particles as sample liquid is moved and to concentrate the magnetic particles within the detection zone. Exemplary incubation times may be, e.g., at least about 0.5 min, 1 min, at least about 2 min, at least about 3 min, at least about 5 min, at least about 10 min, or at least about 12 min. Exemplary incubation times may be about 15 min or less, about 11 min or less, or about 7.5 min or less. This incubation process may be repeated multiple times, e.g., at least 2×, at least about 3×, at least about 5× or at least about 10×.

Diagnostic system 101 uses optical fluorescence to determine the presence of a target but other techniques, e.g., other optical techniques such as absorption or colorimetric may be used as well as non-optical techniques such as electrochemical may also be used. Strip 10, 210, 510, 610 use immunological techniques but non immunological techniques may be used such as enzymatic. Sample liquids other than blood may be used including, e.g., other body fluids such as urine and saliva, as well as body fluids combined with other reagents and liquids such as anticoagulants or buffers.

Exemplary suitable techniques, targets, and sample liquids are disclosed in, e.g., the aforementioned '946 application. Exemplary targets include, for example, pathogens such as viral, fungal, or bacterial pathogens, such as influenza, coronaviruses (e.g., SARS-CoV-2), MRSA, c. diff., flaviviruses, candida, cryptococcus) and antibodies to antigens from said pathogens. Exemplary reagents and methods for determining coronavirus related targets are included in U.S. provisional application nos. 62/992,681 filed Mar. 20, 2020, 63/009,906 filed Apr. 14, 2020, and 63/032,378 filed May 29, 2020, with each of the foregoing titled “Coronavirus Assay” and incorporated herein in their entireties. Exemplary reagents and methods for determining pathogens, e.g., viral related targets such as coronavirus and dengue related targets are disclosed in UK Patent Application No. 2006306.1, filed Apr. 29, 2020, titled “Infectious Disease Assay”, which is incorporated herein by reference in its entirety. Such reagents and methods as disclosed in the aforementioned applications may be used or performed in conjunction with the strips, readers, systems and methods disclosed herein.

In embodiments, a strip includes a lysing reagent that comprises a sufficient amount of an exonuclease to release a viral protein (e.g., nucleocapsid protein) from RNA of the virus. Releasing the protein from the RNA increases the amount of protein available to participate in a reaction (e.g., an immunological reaction) to determine the presence of the protein in a sample. Exemplary protein targets include nucleoproteins (e.g. nucleocapsid) of HIV and coronaviruses (e.g., SARS-CoV-2). An exemplary exonuclease is Benzonase® nuclease.

In embodiments, lysing may be performed in the presence of a salt concentration of at least about 0.2 M, at least about 0.3 M, or at least about 0.4 M. The salt concentration may be about 1.2 M or less, about 1.1 M or less, about 1.0 M or less, or about 0.9 M or less. Exemplary salts include chloride salts such as sodium or potassium chloride and combinations thereof.

In embodiments, a strip includes an integrity monitoring reagent configured to determine whether the strip has been exposed to ambient atmosphere or humidity conditions indicative of a failure of the hermetically sealed pouch and/or the exposure of the sealed pouch to excess temperatures. Typically, the integrity monitoring reagent is disposed within a separate channel or chamber disposed within the strip in similar fashion to the microfluidic channel network, but separated therefrom so as not to contaminate the sample liquid or analytic reagents. The channel or chamber has a vent or other opening to expose the integrity monitoring reagent to the gas within the pouch. The reader is configured to monitor the integrity monitoring reagent as with fluorescence or colorimetry to determine a change indicative of adverse environmental conditions or hermetic failure of the pouch.

### EXAMPLES

The following Examples are merely illustrative and are not intended to limit the scope or content of the invention in any way.

### Example 1: SARS-CoV-2 Ab Assay

A diagnostic system as disclosed herein, including a test strip and reader, was used to perform a SARS-CoV-2 Ab immunofluorescence assay for the qualitative detection of total antibodies to SARS-CoV-2 in human in a blood-based sample liquid, e.g., whole blood (capillary finger stick or venous), plasma or serum. The SARS-CoV-2 Ab assay is intended for use as an aid in identifying individuals with an adaptive immune response to SARS-CoV-2 Ab, indicating recent or prior infection. Results are for the detection of SARS CoV-2 antibodies.

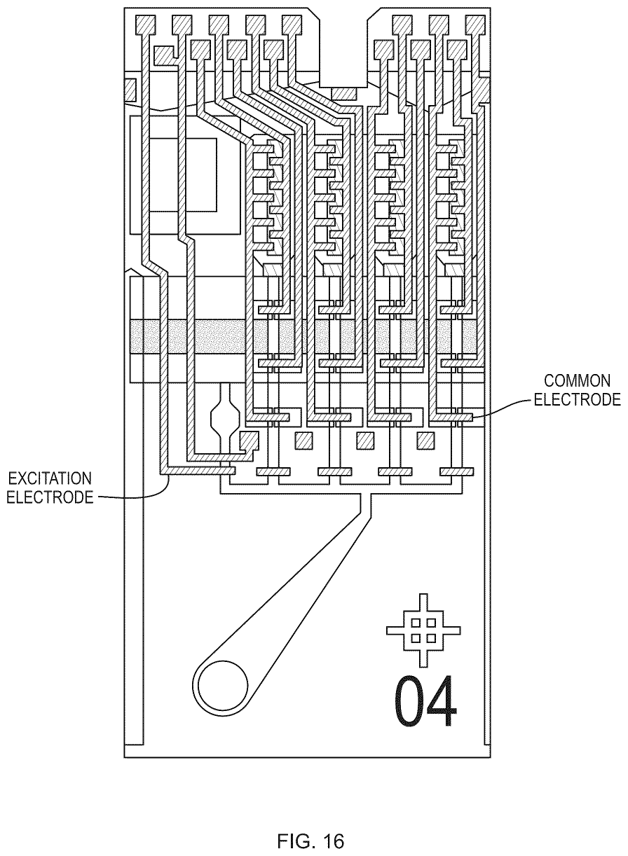

With reference to FIG. 16, the SARS-CoV-2 Ab strip defines a microfluidic channel network having, proceeding upward from lower left, a sample application zone, a tapered common supply channel, a branch channel, and, proceeding from right to left along the branch channel in the Figure, four analysis channels and a hematocrit channel, the proximal portion of which includes an excitation electrode (also referred to as a supply electrode) and a common electrode. As discussed below, the common electrode extends across the hematocrit channel and each of the four analysis channels.

Each of the four analysis channels is arranged and configured to facilitate the determination of the presence and/or amount of the target present in the sample liquid. Proceeding distally from the branch channel along a longitudinal axis of each analysis channel, the analysis channel includes a vent, a capillary stop, a common electrode (common electrode), a reagent zone, a first fill electrode a second fill electrode, a detection zone, a third fill electrode, a spacing channel, and a gas bladder.

In use, a sample is applied to the sample application zone and flows by capillary action along the tapered common supply channel to the branch channel, along which a first portion of the sample liquid flows by capillary action to each of the four analysis channels and a second portion of the sample liquid flows by capillary action to the hematocrit channel. The reader causes the excitation electrode (supply electrode) to generate a time varying signal, e.g., as described for reader 111 and supply electrode 70 of strip 10. If the strip has properly filled with sample liquid, the sample liquid establishes continuity between the excitation electrode and common electrode along each of five pathways: (1) from the portion of the excitation electrode crossing the proximal portion of the hematocrit channel and along the hematocrit channel to the portion of the common electrode crossing the hematocrit channel and (2)-(5) from the portion of the excitation electrode crossing the proximal portion of the hematocrit channel, along the branch channel, and along the proximal portion of each analysis channel to the respective portion of the common electrode crossing such analysis channel. The reader determines the proper filling of the branch channel and the four analysis channels based on the time varying signal measured at the contact of the common electrode at the periphery of the strip. The total impedance between the excitation electrode and the common electrode is smallest when continuity has been established along all five pathways as compared to the total impedance if continuity along one or more pathways has not been established, e.g., if one or more of the analysis channels has not properly filled. Accordingly, the common electrode provides the capability to confirm that each of multiple channels of the strip has properly filled by using only two electrodes (the excitation/supply electrode and the common electrode) and only two contacts at the periphery of the strip (the respective contact corresponding to each electrode).

### SARS-CoV-2 Ab Assay General Principle of Operation

The SARS-CoV-2 Ab Assay uses SARS-CoV-2 specific antigens to form a bridge particle-particle sandwich immunoassay which measures antibodies specific to SARS-CoV-2 present in the test sample.

Dried reagents containing SARS-CoV-2 specific antigen labelled fluorescent particles and SARS-CoV-2 specific antigen labelled biotin are present in dried form within a first reagent zone of each of the four analysis channels. Sample liquid applied to the strip reconstitutes the dried reagents. The reader uses piezo electric actuator to move sample and to mix sample with reagents as described for diagnostic system 101. SARS-CoV-2 antibodies, if present in the sample, form an antigen bridge sandwich complex with the fluorescent particle-labelled and biotin-labelled SARS-CoV-2 antigens. After incubation the resulting immuno-complex is transferred to a detection zone where the reagent is mixed with streptavidin labelled magnetic particles which bind the biotin sandwich complex. A magnetic field is applied to the measurement zone which attracts the magnetic particles and associated SARS-CoV-2 antibody immuno-complexes. The fluidic control system of the reader acting on the strip removes the sample and any unbound label from the measurement zone by piezo electric actuator manipulation (e.g., compression) of the gas bladder at the distal terminus of each analysis channel. Once sample liquid along with unbound label have been removed from the detection zone, the reader measures the fluorescent signal of the immuno-complex fluorescent particles in an essentially dry state which is proportional to the concentration of the SARS-CoV-2 antibody in the sample.

The reader operates the hematocrit channel to facilitate a reagent-free optical determination of the hematocrit of a blood-based sample liquid applied to sample application zone as discussed for strip 10.

### Strip Reagent Configuration

Three of the four analysis channels of the SARS-CoV-2 Ab strip are each used to detect antibodies within the sample liquid. The fourth analysis channel includes on-board-control reagents (OBC) that are used to verify proper assay operation. The SARS-CoV-2 assays are configured using the high specificity antigens of the SARS-CoV-2 virus to ensure high specificity and low cross reactivity. Reagents include the receptor binding domain (RBD) and S1 Spike Glycoprotein (S1) of the SARS-CoV-2 virus.

SARS-CoV-2 (2019-nCoV) Spike S1-His was obtained from Sino Biological Inc. (cat. no. 40591-V08H, Beijing, CN). This protein was constructed by expressing a DNA sequence encoding the SARS-CoV-2 (2019-nCoV) spike protein S1 Subunit (YP_009724390.1) (Val16-Arg685) with a polyhistidine tag at the C-terminus. The Spike S1-His was then conjugated to biotin (A39259, Thermo Fisher Scientific, Waltham MA) or a fluorescent latex particle.

SARS-CoV-2 (2019-nCoV) Spike RBD-mFc was obtained from Sino Biological Inc. (40592-V05H, Beijing, CN). This protein was constructed by expressing a DNA sequence encoding the SARS-CoV-2 (2019-nCoV) Spike Protein RBD (YP_009724390.1) (Arg319-Phe541) with the Fc region of mouse IgG1 at the C-terminus. The Spike RBD-Fc was then conjugated to biotin (A39259, Thermo Fisher Scientific, Waltham MA) or a fluorescent latex particle.

The four-channel strip assay configuration was as follows:

### Analysis Channel 1 S1-S1 Bridge Serology Assay:

SARS-CoV-2 S1 Spike Glycoprotein-Biotin conjugate
SARS-CoV-2 S1 Spike Glycoprotein-Latex conjugate

### Analysis Channel 2 RBD-S1 Bridge Serology Assay:

SARS-CoV-2 S1 Spike Glycoprotein-Biotin conjugate
SARS-CoV-2 Receptor Binding Domain RBD-Latex conjugate

### Analysis Channel 3 RBD-S1 Bridge Serology Assay:

SARS-CoV-2 S1 Spike Glycoprotein-Biotin conjugate
SARS-CoV-2 Receptor Binding Domain RBD-Latex conjugate

### Analysis Channel 4 OBC On Board Control

Biotinylated-Latex conjugate
Streptavidin-Mag Particle conjugate

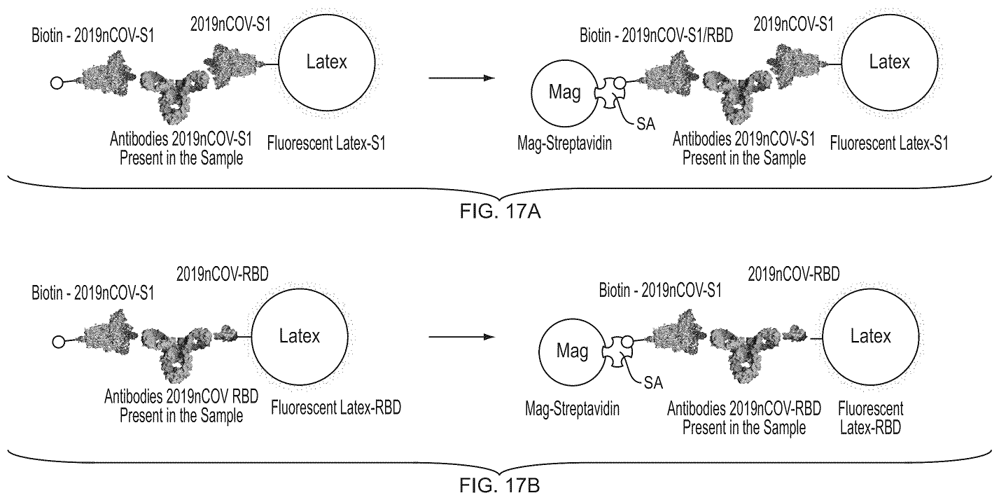

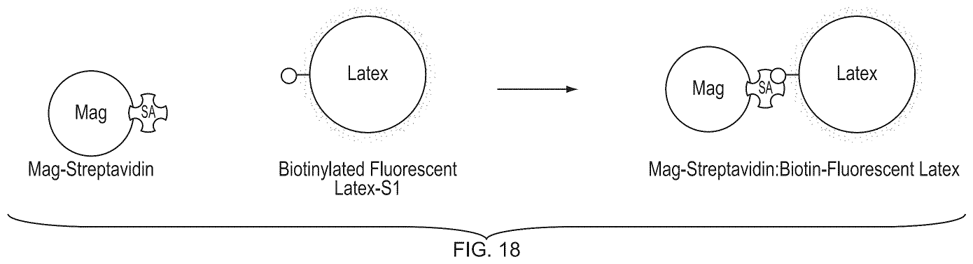

The S1-S1 Bridge and RBD-S1 Bridge Serology assay components and immune-complex formation are respectively illustrated in FIGS. 17A-17B. FIG. 17A illustrates the Bridge Immunoassay, and FIG. 17B illustrates the RBD-S1 Bridge Immunoassay. The On-Board Control assay is illustrated in FIG. 18.

### Operation of Reader and Strip

A user selects the SARS-CoV-2 from the reader menu of assays. The reader performs a self-check to verify the power, electronic, electro-mechanical, and software systems are operating correctly. The user inserts the strip into the reader and applies the liquid sample to the sample application zone of the strip. The liquid sample is a blood-based sample such as whole blood (e.g., finger-stick or venous), plasma, or serum. The reader operates the strip to perform the assays as described for diagnostic system 101, strip 10, 210, 510, or the strip of analysis channel 326.

### Analytical Performance of Assay

### Analytical Sensitivity and Specificity

Reactivity/Inclusivity: Although mutations in the SARS-CoV-2 genome have been identified as the virus has spread, the inventors are not currently aware of serologically unique strains that have been described relative to the originally isolated virus.

Cross-Reactivity: The SARS-CoV-2 Ab Test did not cross react with samples positive for: antibody to Hepatitis C Virus, Hepatitis B Virus (Genotype D) or HIV; human coronaviruses (HKU1, NL63, OC43 and 229E), Anti-Nuclear Antibody, antigen Influenza A, Influenza B, Respiratory Syncytial Virus; heterophile antibodies for mononucleosis. Results are shown in Table 1.

TABLE 1
Cross-reactivity of the SARS-CoV-2 Ab Test

| | Number of | SARS-CoV-2 Ab Test | | |
|---|---|---|---|---|
| Organisms/Conditions | Samples | POS | NEG | % CR |
| Influenza A | 14 | 0 | 14 | 0% |
| Influenza B | 9 | 0 | 9 | 0% |
| Mononucleosis | 5 | 0 | 5 | 0% |
| HCV (IgM and IgG) | 10 | 0 | 10 | 0% |
| HBV (IgM and IgG) | 9 | 0 | 9 | 0% |
| Anti-Nuclear Antibody | 6 | 0 | 6 | 0% |
| HIV (IgM and IgG) | 10 | 0 | 10 | 0% |
| RSV | 7 | 0 | 7 | 0% |
| Human Coronavirus HKU1 | 2 | 0 | 2 | 0% |
| Human Coronavirus NL63 | 1 | 0 | 1 | 0% |
| Human Coronavirus OC43 | 1 | 0 | 1 | 0% |
| Human Coronavirus 229E | 1 | 0 | 1 | 0% |

### Clinical Agreement

i) Positive Agreement

### Endemic, Symptomatic Subjects

Positive agreement was evaluated using plasma samples collected from symptomatic subjects (Table 2). All subjects were confirmed positive for 2019 Novel Coronavirus by RT-PCR. The positive population consisted of the following subjects.
22 Living in the United Kingdom during the 2020 COVID-19 pandemic
52 Living in USA during the 2020 COVID-19 pandemic

TABLE 2
Positive Agreement of the SARS-CoV-2 Ab Test According to Days
Post PCR: Endemic Symptomatic Subjects

| Days from | | | | |
|---|---|---|---|---|
| RT-PCR to | | Number | 2019-nCoV | SARS-CoV-2 Ab Test |
| Blood | | of | RT-PCR | Result as compared to |
| Collection | | Samples | Result | RT-PCR |
| ≤6 | days | 12 | Positive | 10/12 = 83.3% |
| 7-13 | days | 7 | Positive | 7/7 = 100% |
| 14-20 | days | 3 | Positive | 3/3 = 100% |
| >21 | days | 52 | Positive | 52/52 = 100% |

| | | | |
|---|---|---|---|
| Total | 74 | N/A | 72/74 = 97.3% |
| | | | (95% confidence interval: |
| | | | 90.6-99.7%) |

ii) Negative Agreement

### Endemic, Symptomatic Subjects

Negative agreement of the SARS-CoV-2 Ab Test was evaluated using 15 samples (EDTA plasma samples) collected from symptomatic subjects residing in the United Kingdom, shown in Table 3. Samples were collected during the 2020 COVID-19 pandemic and all confirmed negative for 2019 Novel Coronavirus by RT-PCR.

TABLE 3
Negative Agreement of the SARS-CoV-2 Ab Test: Endemic,
Symptomatic Subjects

| | | | SARS-CoV-2 Ab Test |
|---|---|---|---|
| Number of | | 2019-nCoV | Result as compared to |
| Samples | Origin | RT-PCR Result | RT-PCR |
| 15 | UK | Negative | 15/15 = 100% |

### Endemic, Asymptomatic Subjects

In addition, the Negative Agreement of the SARS-CoV-2 Ab Test was evaluated using 22 presumed negative plasma specimens collected from asymptomatic individuals from the United Kingdom during the 2020 COVID-19 pandemic. The resulting negative agreement of the SARS-CoV-2 Ab Test compared to the expected result for all Endemic, Asymptomatic Subjects was 100% (22/22-100%). Results are shown in Table 4 below.

TABLE 4
Negative Agreement of the SARS-CoV-2 Ab Test for Presumed Negative
Endemic, Asymptomatic Subjects

| Number | | | SARS-CoV-2 Ab |
|---|---|---|---|
| of | | 2019-nCoV | Test Result as |
| Samples | Origin | RT-PCR Result | compared to RT-PCR |
| 22 | UK | Unknown | 22/22 = 100% |
| | | (presumed Neg) | |

### Non-Endemic, Asymptomatic Subjects

In addition, the specificity of the SARS-CoV-2 Ab Test was evaluated using 262 presumed negative plasma specimens collected from asymptomatic individuals before the COVID-19 outbreak; thirty three (33) samples were commercially sourced from a Biotechnology Research Service from asymptomatic individuals from the United States collected in 2016, sixty six (66) samples were commercially sourced from a blood donation center, collected in 2019 prior to the COVID-19 pandemic in the United States and one hundred and sixty three (163) were collected during previous clinical evaluations under approved protocols prior to the COVID-19 outbreak from asymptomatic individuals in the United Kingdom (Table 5). All samples were collected between 2016-October 2019. The resulting Negative Agreement of the SARS-CoV-2 Ab Test compared to the expected result was 100% (262/262=100%) and as shown in Table 5.

TABLE 5
Negative Agreement of the SARS-CoV-2 Ab Test: Non-Endemic,
Asymptomatic Subjects

| Number of | | SARS-CoV-2 Ab Test Result as |
|---|---|---|
| Samples | Origin | compared to expected |
| 99 | USA | 99/99 = 100% |
| 163 | UK | 163/163 = 100% |
| Total (262) | N/A | 262/262 = 100% |

### Overall Results

The resulting Negative Agreement of the SARS-CoV-2 Ab Test compared to the expected result was 100% (299/299=100%) with a 95% confidence interval of 98.8 to 100%.

### Example 2: SARS-CoV-2 Ag Assay

A diagnostic system as disclosed herein including a test strip and reader was used to perform a SARS-CoV-2 Ag assay for the qualitative detection of the nucleoprotein antigen to SARS-CoV-2 in nasal and nasopharyngeal swab specimens or after the swabs have been added to either Universal Transport Media (UTM) or Viral Transport Media (VTM) collected from individuals suspected of COVID-19.

Results are for the identification of SARS-CoV-2 nucleoprotein antigen. Antigen is generally detectable in nasal and nasopharyngeal swab during the acute phase of infection.

With reference to FIG. 19, the strip defines a microfluidic channel network having, proceeding upward from lower left, a sample application zone, an arcuate common supply channel, a branch channel, and, proceeding from right to left in the Figure, four analysis channels, a common electrode, an excitation electrode (supply electrode), and a narrow vent channel terminating in a vent (as described for vent channel 576 and vent 576a of microfluidic strip 510).

In use, sample is applied to the sample application zone and flows by capillary action along the arcuate common supply channel to the branch channel along which a first portion of the sample liquid flows by capillary action to each of the four analysis channels and a second portion of the sample liquid flows by capillary action to the excitation/supply electrode, common electrode, and ceases movement at the proximal terminus of the narrow vent channel. The reader causes the excitation electrode (supply electrode) to generate a time varying signal, e.g., as described in Example 1 and for reader 111 and supply electrode 70 of strip 10. If the strip has properly filled with sample liquid, the sample liquid establishes continuity between the excitation electrode and common electrode along each of five pathways: (1) from the left-most portion of the excitation electrode crossing the branch and along the branch channel to the portion of the common electrode crossing the branch channel and (2)-(5) from the portion of the excitation electrode crossing the branch channel, along the branch channel, and along the proximal portion of each analysis channel to the respective portion of the common electrode crossing such analysis channel. As discussed in Example 1, the reader determines the proper filling of the branch channel and the four analysis channels based on the time varying signal measured at the contact of the common electrode at the periphery of the strip.

### SARS-CoV-2 Ag Test Principle

The SARS-CoV-2 Ag assay is a point of care rapid microfluidic immunofluorescence assay. The assay uses SARS-CoV/SARS-CoV-2 specific antibodies in a particle-particle sandwich immunoassay to determine the presence of SARS-CoV-2 Nucleocapsid Protein (NP) present in the test sample.

The reader uses piezoelectric actuators to compress/decompress a gas bladder of each analysis channel to provide liquid movement and mixing of reagents and sample liquid within the microchannel network of the strip. A magnetic field is applied to the measurement zone which captures the magnetic particles and associated SARS-CoV-2 NP immuno-complexes. Before detecting the complexes, the piezoelectric actuator of each channel compresses the corresponding gas bladder to expel liquid sample along with any unbound label from the detection zone. The reader measures the fluorescent signal of the immuno-complex fluorescent particles in an essentially dry state which is proportional to the concentration of the SARS-CoV-2 virus NP antigen in the sample.

### Test Strip Configuration

The SARS-CoV-2 Ag Test uses 2 independent assay channels in the strip to analyze for the NP antigen in the test sample (FIG. 5). A third independent assay channel tests for IgA in the sample. A fourth assay channel comprises the strip on-board control reagents (OBC) that are used to verify the test operated correctly.

The four-channel test strip assay configuration was as follows:

| | |
|---|---|
| Channel 1 | RBD-IgA Serology Assay (Optionally Reported): |
| | SARS-CoV-2 Anti-IgA-Biotin conjugate pre-bound to |
| | Streptavidin-Mag Particle |
| | SARS-CoV-2 Receptor Binding Domain RBD-Latex |
| | conjugate |
| Channel 2 | NP Antigen Assay: |
| | SARS-CoV/SARS-CoV-2 Nucleocapsid Antibody, Mouse |
| | MAb - Latex |
| | SARS-CoV/SARS-CoV-2 Nucleocapsid Antibody, Rabbit |
| | Mab - Mag |
| Channel 3 | NP Antigen Assay: |
| | SARS-CoV/SARS-CoV-2 Nucleocapsid Antibody, Mouse |
| | MAb - Latex |
| | SARS-CoV/SARS-CoV-2 Nucleocapsid Antibody, Rabbit |
| | Mab - Mag |
| Channel 4 | OBC On Board Control (OBC) |
| | Biotinylated-Latex conjugate pre-bound to Streptavidin-Mag |
| | Particle |

The SARS-CoV/SARS-CoV-2 Nucleocapsid Antibody, Mouse Mab was obtained from Sino Biological, Inc. (40143-MM05). The SARS-CoV/SARS-CoV-2 Nucleocapsid Antibody, Rabbit Fab was obtained from LumiraDx UK Ltd. (SD-QMS-WI-30066).

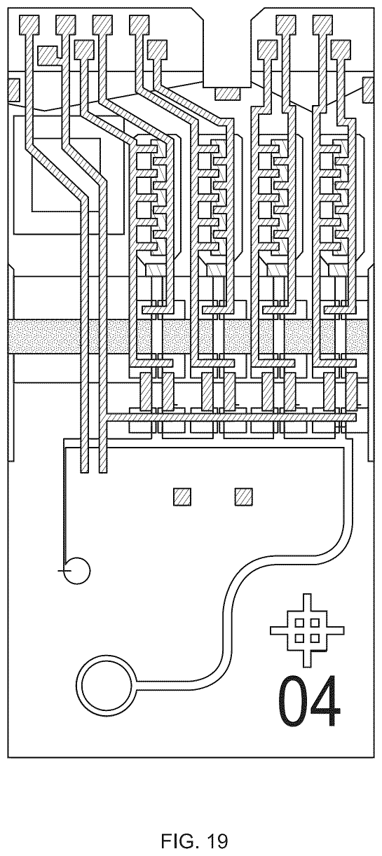

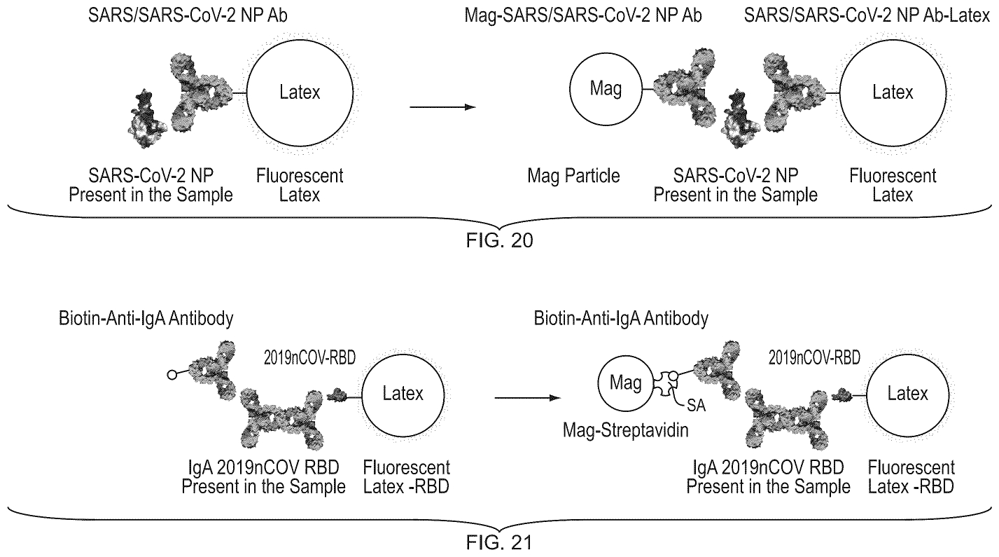

A depiction of the SARS-CoV-2 Ag Nucleocapsid Protein Immunoassay—Channels 2 and 3 is shown in FIG. 20.

A depiction of the RBD-IgA Serology Assay—(Optionally Reported)—Channel 1 is shown in FIG. 21.

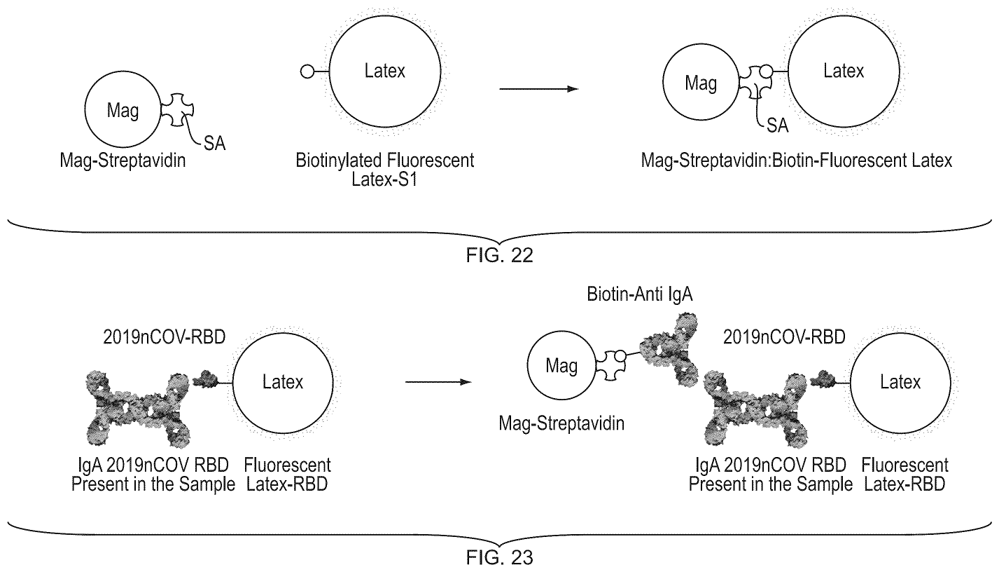

A depiction of the On Board Control Assay—Channel 4 is shown in FIG. 22.

### Operation of Reader and Strip

Preparation and testing of sample proceeded as follows. The liquid sample is a nasal and/or nasopharyngeal swab specimen or such swab specimen that has been combined with Universal Transport Media (UTM) or Viral Transport Media (VTM). A nasal and/or nasopharyngeal swab was obtained from a subject and placed in an extraction buffer. The extraction buffer may be held within an extraction container (vial) as described in U.S. provisional patent application No. 63/029,579 titled “Extraction Container” and filed May 25, 2020, which is incorporated herein in its entirety. For analysis of NP antigen in VTM, the swab is first extracted into VTM and then 700 microliters of VTM is added directly to the extraction buffer container and then stirred by rotating the swab against the side of the vial 5 times. Then, the swab is removed from the extraction vial while squeezing the middle of the extraction container to remove liquid from the swab. The container is sealed with a dropper lid.

With reference to FIG. 23, the schematic for “RBD-IgA Serology Assay—(Optionally Reported)—Channel 1” depicts the formation of an initial complex including (1) an anti-IgA antibody-biotin conjugate (2) anti-SARS-CoV-2 IgA present in a sample, and (3) an RBD-fluorescent latex particle. Next, the initial complex binds to a Mag-streptavidin (capture reagent), which is held in place by the magnet of the reader for fluorescence detection. In the examples that follow, the assay was performed using a strip that included, in dried form, (1) a conjugate comprising an anti-IgA antibody-biotin conjugate pre-bound to a conjugate of streptavidin and a magnetic particle and (2) an RBD-fluorescent latex particle. When a liquid sample was applied to the strip, a complex as shown to the right of the arrow below was formed. The complex was held in place by the magnet of the reader for fluorescence detection.

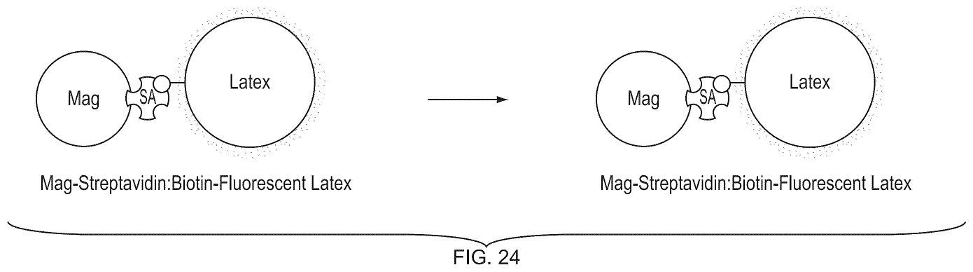

Similarly, for the On Board Control Assay, the strip included, in dried form, (1) a fluorescent latex particle-biotin conjugate pre-bound to a conjugate of streptavidin and a magnetic particle as shown in FIG. 24.

The user selects the SARS-CoV-2 Ag from the reader menu of assays. The reader performs a self-check to verify the power, electronic, electro-mechanical, and software systems are operating correctly. The user inserts the strip into the reader and using the dropper lid applies one drop of the liquid sample to the sample application zone of the strip.

The reader operates the strip to perform the assays as described for diagnostic system 101, strip 10, 210, 510, or the strip of analysis channel 326.

A “calibration LCF” file returns quantitative, un-transformed, final optical signal from each channel on the Instrument screen. Channels 1-3 (looking left to right across a Test Strip) are assay channels while channel 4 is the OBC channel.

The file defines the 4 assays described above. All are displayable. Each assay is assigned 1 calibration curve and to waveband 1, as shown in Table 6.

TABLE 6
Assay summary

| Assay Index | Name, Code, Type | LCF Channel | Result Index |
|---|---|---|---|
| 1 | SARS-CoV-2 IgA, | 4 | 1 |
| | 23, Quantitative | | |
| 2 | SARS-CoV-2 NP- | 3 | 2 |
| | Ag, 25, Quantitative | | |
| 3 | SARS-CoV-2 NP- | 2 | 3 |
| | Ag, 25, Quantitative | | |
| 4 | OBC, 19, | 1 | 4 |
| | Quantitative | | |
| — | SARS-CoV-2 Ag- | Average | 5 |
| | Avg, 26, | output of | |
| | Quantitative | assays 2 & 3 | |

A single calibration curve for each assay is defined for all accepted sample types. In this file all the main channel assay curves and OBC are the same; a simple 1:1 non-transformative calibration table is used in all cases.

An additional displayable Result Index is defined which uses the outputs from assays 2 and 3 to create an average.

Two Quality Control levels are defined (Index 1=Positive, Index 2=Negative) and applied to Result Indexes 1, 2 and 3 though the limits in all cases are 0-1,000,000.

### Limit of Detection (LoD)—Analytical Sensitivity:

### LoD Study 1

LoD studies determine the lowest detectable concentration of SARS-CoV-2 at which approximately 95% of all (true positive) replicates test positive. The LoD of the antigen detection assay as described above was determined by limiting dilution studies using characterized SARS-CoV-2 Culture Fluid Heat Inactivated Virus (Zeptometrix, 0810587CFHI—0.5 ml, Lot 324307).

SARS-Related Coronavirus 2 (Isolate: USA-WA1/2020) is an enveloped, positive-sense single-stranded RNA virus from the Coronaviridae family and the Betacoronaviridae genus. The stock virus was isolated from a patient with a respiratory illness who had returned from travel to the affected region of China and developed COVID-19 in January 2020 in Washington, USA. The genomic sequence can be found in GenBank MN985325.

Each frozen aliquot contains 0.50 mL of heat inactivated viral culture fluid. The pre-inactivation titer was determined from an infectious aliquot. Viral inactivation was verified after heat inactivation by the absence of viral growth in tissue culture-based infectivity assays. (Zeptometrix product description, www.zeptometrix.com/media/documents/PI0810587CFHI—0.5 mL.pdf)

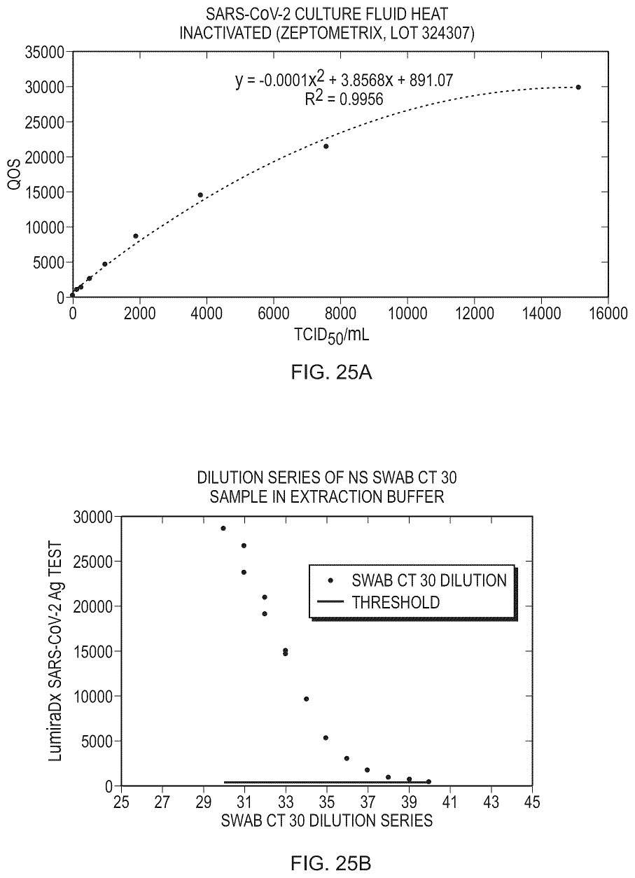

Serial 2-fold dilutions of the characterized SARS-CoV-2 aliquots were tested in 3 replicates. The lowest concentration at which all 3 replicates were positive was treated as the tentative LoD for each test. The LoD of each test was then confirmed by testing 20 replicates with concentrations at the tentative limit of detection. The final LoD of each test was determined to be the lowest concentration resulting in positive detection of 19 out of 20 replicates, as shown in FIG. 25A.

LoD Studies using SARS-CoV-2 Culture Fluid Heat Inactivated Virus (Zeptometrix, 0810587CFHI—0.5 ml, Lot 324307) indicate that the LoD is in the range 1:6400-1:12800 dilution i.e. 118-236 TCID50/ml (median tissue culture infectious dose), as shown in FIG. 25B.

LoD Studies using a dilution series of Patient Nasal/Throat Swab sample (characterized as PCR positive, CT=30; where CT is cycle threshold, defined as the number of cycles required for the fluorescent signal to exceed background level) processed with the SARS-CoV-2 Ag test extraction tube and buffer indicate that the LoD is below 1 in 256 dilution i.e. Ct≤38.

### LoD Study 2

The LoD for the SARS-CoV-2 Ag Test was established using limiting dilutions of gamma-irradiated SARS-CoV-2 (BEI Resources NR-52287). The NR-52287 is a preparation of SARS-Related Coronavirus 2 (SARS-CoV-2), isolate USA-WA1/2020, that has been inactivated by gamma-irradiation at 5×10⁶ RADs. The material was supplied frozen at a concentration of 2.8×10⁵ TCID50/mL.

The study to determine the LoD of the SARS-CoV-2 Ag Test was designed to reflect the assay when using a direct nasal swab. In this study, the starting material was spiked into a volume of pooled human nasal matrix obtained from healthy donors and confirmed negative for SARS-CoV-2. At each dilution, 50 μL samples were added to swabs and the swabs processed for testing on the SARS-CoV-2 Ag Test as per the Package Insert using the procedure appropriate for patient nasal swab specimens. The LoD was determined in 3 steps (following the CLSI Standard, Evaluation of Detection Capability for Clinical Laboratory Measurement Procedures, CLSI EP17):

a. LoD Screening

An initial LoD screening study was performed using a 5-fold serial dilutions (six dilutions in total) of the gamma-irradiated virus made in pooled negative human nasal matrix starting at a test concentration of 2×104 TCID50/mL (as shown in Table 7 below) and processed for each study as described above. These dilutions were tested in triplicate. The lowest concentration at which all (3 out of 3 replicates) were positive was chosen for LoD Range finding. This was 32 TCID50/mL.

TABLE 7
Limit of Detection Analysis for SARS-CoV-2

| SARS-CoV-2 tested | |
|---|---|
| (TCID50/mL) | Test Result |
| 20000 | 3/3 positive |
| 4000 | 3/3 positive |
| 800 | 3/3 positive |
| 160 | 3/3 positive |
| 32 | 3/3 positive |
| 6.2 | 0/3 positive |

b. LoD Range Finding

Using the 32 TCID50/mL concentration, the LoD was further refined using a 2-fold dilution series (four dilutions in total) of the of the gamma-irradiated SARS-CoV-2 virus made in pooled negative human nasal matrix. These dilutions were tested in triplicate. The lowest concentration at which all (3 out of 3 replicates) were positive was treated as the tentative LoD for the SARS-CoV-2 Ag Test. This was 32 TCID50/mL.

TABLE 8
Limit of Detection Analysis for SARS-CoV-2 following
gamma-irradiation

| SARS-CoV-2 tested | |
|---|---|
| (TCID50/mL) | Test Result |
| 32 | 3/3 positive |
| 16 | 0/3 positive |
| 8 | 1/3 positive |
| 4 | 0/3 positive |

c. LoD Confirmation

The LoD of the SARS-CoV-2 Ag Test was then confirmed by testing 20 replicates with concentrations at the tentative Limit of Detection. The final LoD of the SARS-CoV-2 Ag Test was determined to be the lowest concentration resulting in positive detection of twenty (20) out of twenty (20) replicates. Based on this testing the LoD for nasal swab specimens was confirmed as: 32 TCID50/mL

TABLE 9
Summary of Limit of Detection Confirmation Analysis

| Starting Material | | No. | |
|---|---|---|---|
| Concentration | Estimated LOD | Positive/Total | % Positive |
| 2.8 × 10⁵ TCID50/mL | 32 TCID50/mL | 20/20 | 100 |

Cross-reactivity of the SARS-CoV-2 Ag Test was evaluated by testing a panel of related pathogens, high prevalence disease agents and normal or pathogenic flora that are reasonably likely to be encountered in the clinical specimen and could potentially cross-react with the SARS-CoV-2 Ag Test including various microorganisms, viruses and negative matrix. Each organism and virus were tested in the absence or presence of heat inactivated SARS-CoV-2 at 3×LoD. The final concentration of the organisms and viruses are documented in Table 10 below (the concentrations of 10⁶ CFU/mL or higher for bacteria and 10⁵ pfu/mL or higher for viruses is recommended). For a number of microorganisms, the stock concentration was lower than or equal to the recommended testing concentration. In these cases, it was only possible to test these microorganisms at the stock concentration.

TABLE 10
Cross-reactivity analysis of indicated microorganism with
SARS-CoV-2 test

| | | | | Cross- |
|---|---|---|---|---|
| Microorganism | Source | Concentration | | Reactivity |
| Human | Zeptometrix | 1 × 10⁵ | PFU/mL | No |
| coronavirus 229E | | | | (3/3 negative) |
| Human | Zeptometrix | 1 × 10⁵ | PFU/mL | No |
| coronavirus OC43 | | | | (3/3 negative) |
| Human | Zeptometrix | 9.87 × 10³ | PFU/mL | No |
| coronavirus NL63 | | | | (3/3 negative) |
| MERS coronavirus | Zeptometrix | 7930 | PFU/mL | No |
| | | | | (2/2 negative) |
| Adenovirus | Zeptometrix | 1 × 10⁵ | PFU/mL | No |
| (e.g. C1 Ad. 71) | | | | (3/3 negative) |
| Human | Zeptometrix | 1 × 10⁵ | PFU/mL | No |
| Metapneumovirus | | | | (3/3 negative) |
| Parainfluenza virus | Zeptometrix | 1 × 10⁵ | PFU/mL | No |
| Type 1 | | | | (3/3 negative) |
| Parainfluenza virus | Zeptometrix | 1 × 10⁵ | PFU/mL | No |
| Type 2 | | | | (3/3 negative) |
| Parainfluenza virus | Zeptometrix | 1 × 10⁵ | PFU/mL | No |
| Type 3 | | | | (3/3 negative) |
| Parainfluenza virus | Zeptometrix | 1 × 10⁵ | PFU/mL | No |
| Type 4a | | | | (3/3 negative) |
| Influenza A H3N2 | Zeptometrix | 8.82 × 10⁴ | PFU/mL | No |
| | | | | (3/3 negative) |
| Influenza A H1N1 | Zeptometrix | 1 × 10⁵ | PFU/mL | No |
| | | | | (3/3 negative) |
| Influenza B | Zeptometrix | 2.92 × 10⁴ | PFU/mL | No |
| | | | | (3/3 negative) |
| Enterovirus | Zeptometrix | 1 × 10⁵ | PFU/mL | No |
| | | | | (3/3 negative) |
| Respiratory | Zeptometrix | 1 × 10⁵ | PFU/mL | No |
| syncytial virus | | | | (3/3 negative) |
| Rhinovirus | Zeptometrix | 4.17 × 10⁵ | PFU/mL | No |
| | | | | (3/3 negative) |
| Haemophilus | Zeptometrix | 1 × 10⁶ | CFU/mL | No |
| influenzae | | | | (3/3 negative) |
| Streptococcus | Zeptometrix | 1 × 10⁶ | CFU/mL | No |
| pneumoniae | | | | (3/3 negative) |
| Streptococcus | Zeptometrix | 1 × 10⁶ | CFU/mL | No |
| pyogenes | | | | (3/3 negative) |
| Candida albicans | Zeptometrix | 1 × 10⁶ | CFU/mL | No |
| | | | | (3/3 negative) |
| Pooled human | LumiraDx | 14% v/v | | No |
| nasal wash | | | | (3/3 negative) |
| Bordetella | Zeptometrix | 1 × 10⁶ | CFU/mL | No |
| pertussis | | | | (3/3 negative) |
| Mycoplasma | ATCC | 1 × 10⁶ | CFU/mL | No |
| pneumoniae | | | | (3/3 negative) |
| Chlamydia | ATCC | 1 × 10⁶ | CFU/mL | No |
| pneumoniae | | | | (3/3 negative) |
| Legionella | Zeptometrix | 1 × 10⁶ | CFU/mL | No |
| pneumophila | | | | (3/3 negative) |
| Mycobacterium | Zeptometrix | 1 × 10⁶ | CFU/mL | No |
| tuberculosis | | | | (3/3 negative) |
| Pneumocystis | Zeptometrix | 1 × 10⁶ | CFU/mL | No |
| jirovecii | | | | (3/3 negative) |
| Psuedomonas | Zeptometrix | 1 × 10⁶ | CFU/mL | No |
| Aeruginosa | | | | (3/3 negative) |
| Staphylococcus | Zeptometrix | 1 × 10⁶ | CFU/mL | No |
| Epidermidis | | | | (3/3 negative) |
| Streptococcus | Zeptometrix | 1 × 10⁶ | CFU/mL | No |
| Salivarius | | | | (3/3 negative) |

To estimate the likelihood of cross-reactivity of the SARS-CoV-2 Ag Test with organisms that were not available for wet testing, In silico analysis using the Basic Local Alignment Search Tool (BLAST) managed by the National Center for Biotechnology Information (NCBI) was used to assess the degree of protein sequence homology.

For Human Coronavirus HKU1, homology exists between the SARS-CoV-2 nucleocapsid protein and Human Coronavirus HKU 1. BLAST results showed 30 sequence IDs, all nucleocapsid protein, showing homology. Sequence ID AGW27840.1 had the highest alignment score and was found to be 39.1% homologous across 76% of the sequences, this is relatively low but cross-reactivity cannot be fully ruled out.

For SARS-Coronavirus, high homology exists between the SARS-CoV-2 nucleocapsid protein and SARS-Coronavirus. BLAST results showed 68 sequence IDs, mostly nucleocapsid protein, showing homology. Sequence ID AAR87518.1, had the highest alignment score isolated from a human patient and was found to be 90.76% homologous across 100% of the sequence. This is high and cross-reactivity is likely.

For MERS-Coronavirus, high homology exists between the SARS-CoV-2 nucleocapsid protein and MERS-Coronavirus. BLAST results showed at least 114 sequence IDs, mostly nucleocapsid protein, showing homology. Sequence IDs AHY61344.1 and AWH65950.1, had the highest alignment scores isolated from a human patient and were found to be 49.4% and 50.3% homologous across 88% of the sequence. Whilst this potentially represents moderate cross-reactivity testing of the MERS virus at 7930 PFU/mL showed no reactivity (see table above).

### Micrbal Interference Studies

Microbial interference in the SARS-CoV-2 Ag Test was evaluated by testing a panel of related pathogens, high prevalence disease agents and normal or pathogenic flora to demonstrate that false negatives do not occur when SARS-CoV-2 is present in a specimen with other microorganisms including various microorganisms, viruses and negative matrix. Each organism and virus were tested in triplicate in the absence or presence of heat inactivated SARS-CoV-2 at 3×LoD. The final concentration of the organisms and viruses are documented in the Table below (the concentrations of 10⁶ CFU/mL or higher for bacteria and 10⁵ pfu/mL or higher for viruses is recommended). For a number of microorganisms, the stock concentration was lower than or equal to the recommended testing concentration. In these cases, it was only possible to test these microorganisms at the stock concentration.

TABLE 11
Interference analysis of indicated microorganism with
SARS-CoV-2 test

| Microorganism | Source | Concentration | | Interference |
|---|---|---|---|---|
| Human | Zeptometrix | 1 × 10⁵ | PFU/mL | No |
| coronavirus 229E | | | | (3/3 positive) |
| Human | Zeptometrix | 1 × 10⁵ | PFU/mL | No (19/20 |
| coronavirus OC43 | | | | positive) |
| Human | Zeptometrix | 9.87 × 10³ | PFU/mL | No |
| coronavirus NL63 | | | | (3/3 positive) |
| MERS coronavirus | Zeptometrix | 7930 | PFU/mL | No |
| | | | | (3/3 positive) |
| Adenovirus | Zeptometrix | 1 × 10⁵ | PFU/mL | No |
| (e.g. C1 Ad. 71) | | | | (3/3 positive) |
| Human | Zeptometrix | 1 × 10⁵ | PFU/mL | No |
| Metapneumovirus | | | | (3/3 positive) |
| Parainfluenza virus | Zeptometrix | 1 × 10⁵ | PFU/mL | No |
| Type 1 | | | | (3/3 positive) |
| Parainfluenza virus | Zeptometrix | 1 × 10⁵ | PFU/mL | No |
| Type 2 | | | | (3/3 positive) |
| Parainfluenza virus | Zeptometrix | 1 × 10⁵ | PFU/mL | No |
| Type 3 | | | | (3/3 positive) |
| Parainfluenza virus | Zeptometrix | 1 × 10⁵ | PFU/mL | No |
| Type 4a | | | | (3/3 positive) |
| Influenza A H3N2 | Zeptometrix | 8.82 × 10⁴ | PFU/mL | No |
| | | | | (3/3 positive) |
| Influenza A H1N1 | Zeptometrix | 1 × 10⁵ | PFU/mL | No |
| | | | | (3/3 positive) |
| Influenza B | Zeptometrix | 2.92 × 10⁴ | PFU/mL | No (19/20 |
| | | | | positive) |
| Enterovirus | Zeptometrix | 1 × 10⁵ | PFU/mL | No |
| | | | | (3/3 positive) |
| Respiratory | Zeptometrix | 1 × 10⁵ | PFU/mL | No |
| syncytial virus | | | | (3/3 positive) |
| Rhinovirus | Zeptometrix | 4.17 × 10⁵ | PFU/mL | No |
| | | | | (3/3 positive) |
| Haemophilus | Zeptometrix | 1 × 10⁶ | CFU/mL | No |
| influenzae | | | | (3/3 positive) |
| Streptococcus | Zeptometrix | 1 × 10⁶ | CFU/mL | No |
| pneumoniae | | | | (3/3 positive) |
| Streptococcus | Zeptometrix | 1 × 10⁶ | CFU/mL | No |
| pyogenes | | | | (3/3 positive) |
| Candida albicans | Zeptometrix | 1 × 10⁶ | CFU/mL | No |
| | | | | (3/3 positive) |
| Pooled human | LumiraDx | 14% v/v | | No |
| nasal wash | | | | (3/3 positive) |
| Bordetella | Zeptometrix | 1 × 10⁶ | CFU/mL | No |
| pertussis | | | | (3/3 positive) |
| Mycoplasma | ATCC | 1 × 10⁶ | CFU/mL | No |
| pneumoniae | | | | (3/3 positive) |
| Chlamydia | ATCC | 1 × 10⁶ | CFU/mL | No |
| pneumoniae | | | | (3/3 positive) |
| Legionella | Zeptometrix | 1 × 10⁶ | CFU/mL | No |
| pneumophila | | | | (3/3 positive) |
| Mycobacterium | Zeptometrix | 1 × 10⁶ | CFU/mL | No |
| tuberculosis | | | | (3/3 positive) |
| Pneumocystis | Zeptometrix | 1 × 10⁶ | CFU/mL | No |
| jirovecii | | | | (3/3 positive) |
| Psuedomonas | Zeptometrix | 1 × 10⁶ | CFU/mL | No |
| Aeruginosa | | | | (3/3 positive) |
| Staphylococcus | Zeptometrix | 1 × 10⁶ | CFU/mL | No |
| Epidermidis | | | | (3/3 positive) |
| Streptococcus | Zeptometrix | 1 × 10⁶ | CFU/mL | No |
| Salivarius | | | | (3/3 positive) |

### Endogenous Interference Substances Studies

A study was performed to demonstrate that twenty two (22) potentially interfering substances that may be found in the upper respiratory tract in symptomatic subjects (including over the counter medications) do not cross-react or interfere with the detection of SARS-CoV-2 in the SARS-CoV-2 Ag Test. Each substance was tested in triplicate in the absence orpresence ofSARS-CoV-2 at 3×LoD. Substances for testing were selected based on the respiratory specimens guidance at the world wide web at accessdata.fda.gov/cdrh_docs/reviews/K112177.pdf.

The final concentration of the substances tested are documented in Table 12 below.

TABLE 12
Interference analysis of indicated substances with SARS-CoV-2 test

| | | | Interference |
|---|---|---|---|
| Interfering Substance | Concentration | | (Yes/No) |
| Benzocaine | 150 | mg/dL | No (3/3 Negative, 3/3 |
| Blood (human) | 5% | | No (3/3 Negative, 3/3 |
| Mucin | 5 | mg/mL | No (3/3 Negative, 3/3 |
| Naso GEL (NeilMed) | 5% | v/v | No (3/3 Negative, 3/3 |
| CVS Nasal Drops | 15% | v/v | No (3/3 Negative, 3/3 |
| (phenylephrine) | | | |
| Afrin (Oxymetazoline) | 15% | v/v | No (3/3 Negative, 3/3 |
| CVS Nasal Spray | 15% | v/v | No (3/3 Negative, 3/3 |
| (Cromolyn) | | | |
| Zicam Cold Remedy | 5% | v/v | No (3/3 Negative, 3/3 |
| Homeopathic (Alkalol) | 10 % | v/v | No (3/3 Negative, 3/3 |
| Sore Throat Phenol | 15% | v/v | No (3/3 Negative, 3/3 |
| Spray | | | |
| Tobramycin | 3.3 | mg/dL | No (3/3 Negative, 3/3 |
| Mupirocin | 0.15 | mg/dL | No (3/3 Negative, 3/3 |
| Fluticasone | 0.000126 | mg/dL | No (5/5 Negative, 4/4 |
| Tamiflu | 500 | mg/dL | No (3/3 Negative, 3/3 |
| (Oseltamivir phosphate) | | | |
| Budenoside | 0.00063 | mg/dL | No (3/3 Negative, 3/3 |
| Biotin | 0.35 | mg/dL | No (3/3 Negative, 3/3 |
| Methanol | 150 | mg/dL | No (19/20 Negative, |
| | | | 3/3 Positive) |
| Acetylsalicylic Acid | 3 | mg/dL | No (3/3 Negative, 3/3 |
| Diphenhydramine | 0.0774 | mg/dL | No (3/3 Negative, 3/3 |
| Dextromethorphan | 0.00156 | mg/dL | No (19/20 Negative, |
| | | | 3/3 Positive) |
| Dexamethasone | 1.2 | mg/dL | No (3/3 Negative, 3/3 |

| | | |
|---|---|---|
| Mucinex | 5% | No (3/3 Negative, 3/3 |

### High Dose Hook Effect

High Dose Hook Effect studies determine the level at which false negative results can be seen when very high levels of target are present in a tested sample. To determine if the SARS-CoV-2 Ag Test suffers from any high dose hook effect, increasing concentrations of gamma-irradiated SARS-CoV-2 virus (BEI OResources NR-52287) were tested up to a concentration of 1.4×10⁵ TCID50/mL. In this study, the starting material was spiked into a volume of pooled human nasal matrix obtained from healthy donors and confirmed negative for SARS-CoV-2. At each dilution, 50 μL samples were added to swabs and the swabs processed for testing on the SARS-CoV-2 Ag Test as per the Package Insert using the procedure appropriate for patient nasal swab specimens. Samples were tested in triplicate.

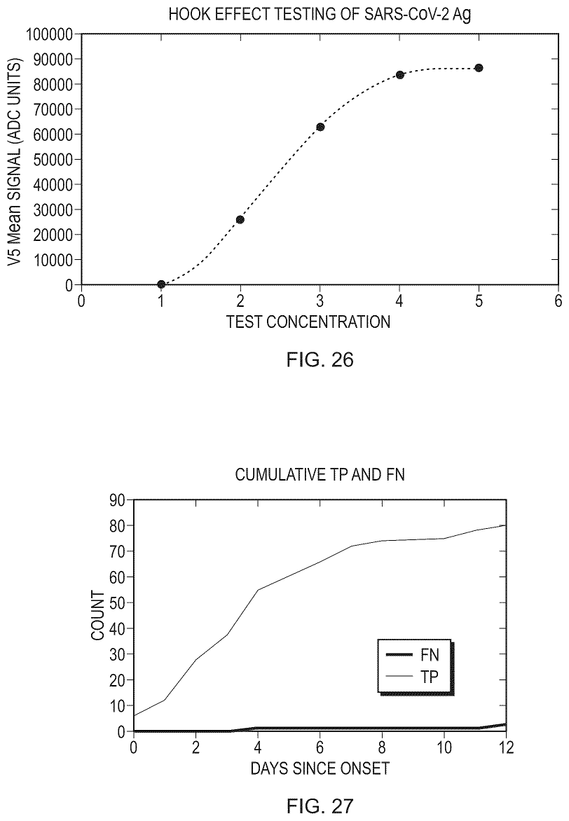

No impact on test performance or high dose hook effect was observed up to 1.4×10 TCID50/mL of gamma-irradiated SARS-CoV-2 with the SARS-CoV-2 Ag Test as shown in Table 13 and FIG. 26.

TABLE 13
Analysis of High Dose Hook Effect

| | Concentration | |
|---|---|---|
| Test Dilution | (TCID50/mL) | Mean Signal (ADC Units) |
| 1 | 0 | 495 |
| 2 | 62.5 | 26100.6 |
| 3 | 250 | 63013.8 |
| 4 | 1000 | 83451.8 |
| 5 | 1.4 × 10⁵ | 86220 |

### Clinical Performance

The performance of the SARS-CoV-2 Ag Test was established with 294 nasal or nasal-throat swabs prospectively collected from a total of 357 individual subjects during the 2020 COVID-19 pandemic. Subjects were either presenting with symptoms of COVID-19 (194) or key workers (100) being screened for infection. Samples were collected from 9 sites across the United States (6) and United Kingdom (3). Swabs were collected and extracted into extraction buffer (Tauns Laboratories, Inc.). Specimens were tested fresh or frozen within 1 h of collection and stored until tested. No sample concentration was performed. Samples were thawed and sequentially tested according to the Product Insert, with operators blinded to the PCR result. The performance of the SARS-CoV-2 Ag Test was compared to the results from nasal swabs or nasal-throat samples collected into 3 ml universal transport medium (UTM) and tested with an EUA-authorized PCR method (Cobas® SARS-CoV test using the Cobas® 6800 PCR analyzer). Data analysis is presented in Table 14.

TABLE 14
Comparison of SARS-CoV-2 Ag Test and RT-PCR Assay for SARS-CoV-2

| | | | | | | | 95% Wilson | |
|---|---|---|---|---|---|---|---|---|
| Reference RT-PCR Assay | | | | | | | Score CI | |
| | | POS | NEG | TOTAL | | Est | LCI | UCI |
| LumiraDx | POS | 81 | 6 | 87 | PPA | 97.6% | 91.6% | 99.3% |
| SARS- | NEG | 2 | 168 | 170 | NPA | 96.6% | 92.7% | 98.4% |
| CoV-2 | TOTAL | 83 | 174 | 257 | PPV | 93.1% | 85.8% | 96.8% |
| Ag Test | | | | | NPV | 98.8% | 95.8% | 99.7% |
| | | | | | Prevalence | 32.3% | 26.9% | 38.2% |
| | | | | | OPA | 96.9% | 94.0% | 98.4% |

PPA—Positive Percent Agreement; NPA—Negative Percent Agreement; OPA—Overall Percent Agreement; PPV—Positive Predictive Value; NPV—Negative Predictive Value; CI—Confidence Interval.

FIG. 27 shows the cumulative Positives and False Negatives for the LumiraDx Ag test over a 12 days period since symptom onset.

Table 15 shows the cumulative sensitivity of the SARS-CoV-2 Ag Test over time with 95% Wilson Score Confidence Interval (CI).

TABLE 15
Analysis of Sensitivity for SARS-CoV-2 Ag and RT-PCR tests

| Days Since | Cumulative | | | | |
|---|---|---|---|---|---|
| Symptom | PCR | LumiraDx | Sensitivity | | |
| Onset | Positive (+) | Positive (+) | (PPA) | LCI | UCI |
| 0 | 6 | 6 | 100.0% | 61.0% | 100.0% |
| 1 | 12 | 12 | 100.0% | 75.8% | 100.0% |
| 2 | 28 | 28 | 100.0% | 87.9% | 100.0% |
| 3 | 37 | 37 | 100.0% | 90.6% | 100.0% |
| 4 | 55 | 54 | 98.2% | 90.4% | 99.7% |
| 5 | 61 | 60 | 98.4% | 91.3% | 99.7% |
| 6 | 67 | 66 | 98.5% | 92.0% | 99.7% |
| 7 | 73 | 72 | 98.6% | 92.6% | 99.8% |
| 8 | 75 | 74 | 98.7% | 92.8% | 99.8% |
| 10 | 77 | 76 | 98.7% | 93.0% | 99.8% |
| 11 | 80 | 79 | 98.8% | 93.3% | 99.8% |
| 12 | 83 | 81 | 97.6% | 91.6% | 99.3% |

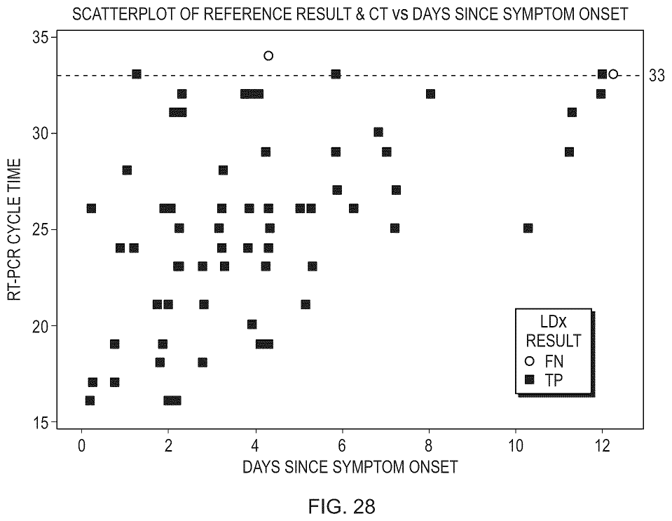

FIG. 28 shows a plot of RT-PCR cycle time (“Ct”) for samples collected a given number of days after symptom onset. The scatterplot shows only the portion of the data for which both (1) days since symptom onset and (2) Ct values (PCR data) as available.

The above data shows that the relatively large data set together with PCR test Ct values allows a true sensitivity comparison between the SARS-CoV-2 Ag test and PCR. The sensitivity of the SARS-CoV-2 Ag test is 97.6. Sensitivity from day 5 post symptom onset is 27/28 (96.4% with a CI of 82.3 to 99.4%). This compares to a sensitivity of 54/55 (98.2% with CI 90.4 to 99.7%) for samples at day 4 or earlier post symptom onset. These CI overlap and thus there is not a large drop off after the early days. Sensitivity is determined by viral load and therefore the Limit of Detection of the test. The data clearly shows that, due to its high sensitivity, the SARS-CoV-2 Ag test is effective across the full 12-day period of data collection.

The data shows a cut off at around Ct 33/34 which is consistent across the data set, independent of days since symptom onset, which may indicate that viral load above Ct 33/34 is rare in mildly symptomatic patients and potentially indicating cessation of the infection. This is in agreement with a number of recently published papers (Wolfel et al. (2020) Nature 581:465-469; McIntosh et al. (2020) at www.uptodate.com/contents/coronavirus-disease-2019-covid-19-epidemiology-virology-and-prevention).

The two False Negatives in the data set are just below the test threshold value (>33Ct) and appear random rather than being time related. Due to its high sensitivity, the SARS-CoV-2 Ag test, (LOD 32 TCID50/ml) correctly identifies every positive patient with Ct<33. Based on the reported LOD values of other SARS-CoV-2 antigen tests, it appears that those tests likely would fail to identify any Ct>30. Based on this data set, a test that fails to identify any Ct>30 translates into a comparative sensitivity of approximately 80% (51/65).

### SEQUENCE LISTING

SEQ ID NO: 1
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKENGLTVLPPLLTDEMIAQYTSALLAGTI
TSGWTFGAGAALQIPFAMQMAYRENGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

1.-271. (canceled)

272. A microfluidic device, comprising:
a microfluidic channel network comprising a microchannel configured to receive a liquid and a mechanical manipulation zone in fluidic communication with the microchannel, the mechanical manipulation zone comprising an upper wall and a lower wall disposed in opposition to one another and wherein a mechanical compression or decompression of one of the upper or lower walls with respect to the other of the upper and lower walls induces movement of a liquid if present in the microchannel;
a first electrode disposed to contact liquid within the microchannel at a first location;
a first electrically conductive lead extending from the first electrode to a first electrical contact disposed on the microfluidic device outside of the microfluidic channel network, the first electrically conductive lead comprising a first lead portion disposed within, or adjacent, the mechanical manipulation zone;
a second electrode disposed to contact liquid within the microchannel at a second location spaced apart from the first location;
a second electrically conductive lead extending from the second electrode to a second electrical contact disposed on the microfluidic device outside of the microfluidic channel network and spaced apart from the first electrical contact, the second electrically conductive lead comprising a second lead portion disposed within, or adjacent, the mechanical manipulation zone;
wherein, the first and second electrodes are each configured to perform a respective liquid sensing or target detection function at the respective first and second location and a mechanical compression of one of the upper or lower walls with respect to the other of the upper and lower walls brings the first and second electrical contacts into electrical communication with one another and, if the first and second lead portions are in electrical communication with one another, a mechanical decompression of one of the upper or lower walls with respect to the other breaks the electrical communication between the first and second electrical contacts.

273. The microfluidic device of claim 272, wherein the mechanical manipulation zone is a gas bladder in fluid communication with the microchannel.

274. The microfluidic device of claim 273, wherein the first and second lead portions define a plurality of interposed first and second lead portions.

275. The microfluidic device of claim 274, wherein the lead portions of the first and second interposed lead portions are spaced apart from one another along a longitudinal axis of the gas bladder.

276. The microfluidic device of claim 275, wherein the first and second lead portions are each disposed on an interior surface of one of the upper and lower walls of the mechanical manipulation zone and an interior of the other of the upper and lower walls comprises an electrically conductive surface configured to bring the first and second lead portions into electrical communication with one another upon compression of the mechanical manipulation zone.

277. The microfluidic device of claim 276, wherein the electrically conductive surface is a surface of an electrically conductive bridging member secured to the interior of the other of the upper and lower walls.

278. The microfluidic device of claim 277, wherein the electrically conductive bridging member is disposed to contact a plurality of the lead portions of the plurality of first and second lead portions upon compression of the gas bladder.

279. The microfluidic device of claim 272, wherein the microfluidic device comprises a supply electrode disposed to contact liquid within the microchannel at a third location spaced apart from each of the first and second locations, and further wherein the first and second electrodes are each configured to perform a respective liquid sensing function at the respective first and second locations by detecting a signal emitted by the supply electrode.

280. The microfluidic device of claim 272, wherein the microfluidic device is configured to perform a diagnostic assay.

281. The microfluidic device of claim 280, wherein the microfluidic device comprises a reagent configured for use in the diagnostic assay disposed within the microchannel.

282. The microfluidic device of claim 281, wherein the reagent is disposed within the microchannel between the first and second locations.

283. The microfluidic device of claim 280, wherein the reagent is selected from the group consisting of a lysing reagent for lysing a cell, a buffering reagent, a detectably labeled reagent, a reagent configured to specifically bind a target to be detected, a magnetically labeled reagent, or a combination thereof.

284. A method of operating a microfluidic device, comprising:
placing the microfluidic device in an operative position with respect to a diagnostic reader, the microfluidic device comprising (a) a microfluidic channel network, the microfluidic network comprising an application zone, a microchannel configured to receive a liquid applied to the application zone, and a gas bladder in gaseous communication with the microchannel and (b) (i) a first electrical contact disposed on the microfluidic device outside of the microfluidic channel network and in electrical communication with a first lead portion disposed within or adjacent the gas bladder and a first liquid sensing electrode disposed to contact liquid within the microchannel at a first location and (ii) a second electrical contact disposed on the microfluidic device outside of the microfluidic channel network and in electrical communication with a second lead portion disposed within or adjacent the gas bladder and a second liquid sensing electrode disposed to contact liquid within the microchannel at a second location spaced apart from the first location, wherein the first and second electrical contacts are in electrical communication with one another when the gas bladder is in a fully compressed state;
compressing a wall of the gas bladder until the gas bladder is in the fully compressed state with the first and second electrical contacts in electrical communication with one another;
applying a liquid sample to an application zone of the microfluidic channel network;
decompressing the wall of the gas bladder thereby moving the liquid sample within the microfluidic channel network; and
after the step of decompressing the wall of the gas bladder, sensing the presence of the liquid sample at the first liquid sensing electrode by detecting a first electrical signal at the first electrical contact and sensing the presence of the liquid sample at the second liquid sensing location by detecting a second electrical signal at the second electrical contact.

285. The method of claim 284, wherein the step of decompressing the wall of the gas bladder is performed after the step of applying the liquid sample to the application zone.

286. The method of claim 284, wherein the microfluidic device comprises a supply electrode disposed to contact liquid sample within the microfluidic channel network at a third location spaced apart from the first and second locations and further wherein the first and second electrical signals each comprise an electrical signal emitted by the supply electrode.

287. The method of claim 284, wherein the first and second lead portions define a plurality of interposed first and second lead portions.

288. The method of claim 287, wherein the lead portions of the first and second interposed lead portions are spaced apart from one another along a longitudinal axis of the gas bladder.

289. The method of claim 288, wherein the wall of the gas bladder is a first wall and the gas bladder comprises a second wall opposing the first wall, the first and second lead portions are each disposed on an interior surface of one of the first and second walls of the gas bladder, an interior of the other of the first and second walls of the gas bladder comprises an electrically conductive bridging member, and the step of compressing the first wall of the gas bladder comprises bringing the bridging member and the first and second lead portions into electrical communication with one another.

290. The method of claim 289, wherein the step of compressing the first wall of the gas bladder comprises bringing the bridging member into electrical communication with each of a plurality of the first and second interposed lead portions.

291. The method of claim 284, wherein the method comprises performing a diagnostic assay to detect a target present in the liquid sample.

292. The method of claim 291, wherein performing the diagnostic assay comprises contacting the liquid sample with a reagent disposed in the microchannel.

293. The method of claim 292, wherein the step of contacting the liquid sample with the reagent is performed after sensing the presence of the liquid sample at the first liquid sensing electrode and prior to sensing the presence of the liquid sample at the second liquid sensing location.

294. The method of claim 292, wherein the reagent is selected from the group consisting of a lysing reagent for lysing a cell, a buffering reagent, a detectably labeled reagent, a reagent configured to specifically bind the target, a magnetically labeled reagent, or a combination thereof.
